(19) United States
(12) Patent Application Publication
(10) Pub. No.: US 2026/0191908 A1
(43) Pub. Date: Jul. 9, 2026

(54) COMPOSITIONS AND METHODS FOR TREATING GENETIC DISORDERS

(71) Applicants: Centro de Investigaciones Energéticas, Medioambientales y Tecnológicas, O.A, M.P., Madrid (ES); Consorcio Centro de Investigación Biomédica en Red, M.P., Madrid (ES); Fundación Instituto de Investigación Sanitaria Fundación Jiménez Díaz, Madrid (ES); Fundación para la Investigación Biomédica del Hospital Infantil Universitario Niño Jesús, Madrid (ES)

(72) Inventors: Juan Antonio Bueren RONCERO, New York, NY (US); Paula Rio GALDO, New York, NY (US); Susana Navarro Ordóñez, New York, NY (US); Julian Sevilla NAVARRO, New York, NY (US); Jose Carlos Segovia SANZ, New York, NY (US); Africa Gonzalez MURILLO, New York, NY (US); Jose Antonio Casado OLEA, New York, NY (US); Guillermo Güenechea AMURRIO, New York, NY (US)

(21) Appl. No.: 19/392,021

(22) Filed: Nov. 17, 2025

Related U.S. Application Data
(63) Continuation of application No. 16/331,455, filed on Sep. 8, 2017, now abandoned.
(60) Provisional application No. 62/385,185, filed on Sep. 8, 2016.
(60) Provisional application No. 62/412,028, filed on Oct. 24, 2016.

(51) Int. Cl. A61K 35/28; A61K 38/17; A61K 48/00; A61P 7/06; C12N 15/86

(52) U.S. Cl. CPC A61K 35/28; A61K 38/1709; A61K 48/0066; A61P 7/06; C12N 15/86; C12N 2740/15043

(57) ABSTRACT

The present invention provides compositions and methods for rescuing FANCA expression in cells with diminished or no FANCA gene product. In particular, methods and compositions for treating genetic disorders are disclosed.

### FIELD OF THE INVENTION

The present invention relates generally to gene transfer into cells with diminished or no protein activity from one or more FANCA encoded proteins.

### STATEMENT REGARDING SEQUENCE LISTING

The contents of the electronic sequence listing (0203US.xml; Size: 46,794 bytes; and Date of Creation: Nov. 17, 2025) are herein incorporated by reference in their entirety.

### BACKGROUND OF THE INVENTION

Fanconi Anemia (FA) is an autosomal recessive disease (except for complementation group FA-B, which is X-linked), where the median survival of patients is around 24 years (Butturini A, et al. (1994) Blood 84:1650-1655; Kutler D I, et al. (2003) Blood 101:1249-1256). At birth, the blood count of these patients is generally normal. Macrocytosis is often the first hematological abnormality detected in these patients. This usually evolves with thrombocytopenia, anemia and pancytopenia. Bone marrow failure (BMF) is usually observed in these patients after 5-10 years, with an average age of hematologic disease onset of 7 years. About 80% of patients with FA will develop evidence of BMF in the first decade of life. Based on epidemiological studies to date, if malignant episodes do not appear before aplasia, virtually all patients with FA will develop BMF by 40 years of age (Butturini A, et al. (1994) Blood 84:1650-1655; Kutler D I, et al. (2003) Blood 101:1249-1256), this being the leading cause of mortality in these patients.

Due to the complex clinical manifestations of FA, management of these patients is mainly focused on improving the following syndromes: bone marrow failure (BMF), myeloid leukemia, and solid tumors.

Current treatments include androgens such as those composed of fluoxymesterone, oxymetholone or stanozolol. As recently reviewed by Dufour and colleagues (Dufour, C. and Svahn, J. (2008). Bone Marrow Transplant 41 Suppl 2: S90-95), FA patients may have some response to androgens as long as this treatment is not initiated at a very advanced stage of the disease. About 75% of patients respond to androgens. A combination with 2 mg/kg/day of prednisolone reduces the risk of liver toxicity. In general, in the absence of a suitable bone marrow donor, androgens can be considered as an alternative treatment when there is some residual haematopoiesis, but not as a definitive long-term treatment.

Tischkowitz et al. noted that although in the early stages of the disease FA patients might respond to androgens, the vast majority of FA patients are refractory to these treatments in the long term (Tischkowitz, M. and Dokal, I. (2004). Br J Haematol 126: 176-191).

There are no specific indications for hematopoietic growth factors in the treatment of FA. However, two pharmacological groups of drugs with indications for specific symptoms of FA (anemia and neutropenia) have been identified: 1) erythropoietin and 2) granulocyte-colony stimulating factors (G-CSFs). Several erythropoietines (e.g., Aranesp, Nespo, Exjade) are approved for the treatment of anemia, and G-CSFs (e.g., G-CSF analogs such as filgrastim, biogastrin, neulasta) are approved for the treatment of neutropenia. Although hematopoietic growth factors, such as erythropoietin and granulocyte colony-stimulating factors, have been tested in a limited number of patients with FA, responses were partial and transient (Dufour et al., 2008). At present, these treatments do not represent good long-term options. For short-term treatment in neutropenic patients, G-CSF can be used for acute infections to increase the number of peripheral neutrophils, potentiating antibiotics. However, these drug treatments are far from definitive management for patients with FA.

Presently, the only curative treatment of the hematological manifestations of the disease is based on allogeneic hematopoietic transplantation. While the outcome of FA patients transplanted with grafts from HLA-identical sibling donors is in general satisfactory, only about 20% of FA patients will have an HLA-identical sibling. A significant proportion of FA patients without a sibling donor can be transplanted from alternative donors, although these transplants are associated with a higher morbidity and mortality. In the remaining FA patients, no alternative therapies are currently available.

Accordingly, there remains a critical need for an effective treatment regimen for FA. The present invention addresses this need and more.

### SUMMARY OF THE INVENTION

Embodiments of the present invention comprise polynucleotide cassettes for the enhanced expression of FANCA. In some embodiments, the polynucleotide cassette comprises a sequence encoding a codon-optimized human FANCA cDNA to increase mRNA stability upon transcription.

In one embodiment, the present invention includes an expression cassette comprising a polynucleotide sequence comprising in the following 5′ to 3′ order: (a) a human phosphoglycerate kinase (PGK) promoter sequence or a functional homolog or variant thereof; (b) a sequence encoding a human FANCA polypeptide or a functional fragment or variant thereof; (c) a woodchuck hepatitis virus regulatory element (WPRE) RNA export signal sequence or a functional variant or fragment thereof, wherein the sequence encoding the human FANCA polypeptide or functional fragment or variant thereof is operably linked to the PGK promoter sequence. In particular embodiments, the FANCA polypeptide or functional fragment or variant thereof comprises the sequence set forth in SEQ ID NO: 25; the sequence encoding the FANCA polypeptide or functional fragment or variant thereof comprises the sequence set forth in SEQ ID NO: 8; the PGK promoter comprises a nucleotide sequence of SEQ ID NO: 7; and/or the WPRE element comprises a nucleotide sequence of SEQ ID NO: 23. In particular embodiments, the cassette comprises a region of the nucleotide sequence of SEQ ID NO: 24. In certain embodiments, the cassette further comprises one or more enhancer sequences, a polypurine tract (PPT) or polyadenylation (polyA) signal sequence, a packing signal sequence, a truncated Gag sequence, a Rev responsive element (RRE; a central polypurine tract (cPPT), a central terminal sequence (CTS) and/or an upstream sequence element (USE), optionally from simian virus 40 (SV40-USE).

In one embodiment, the present invention provides an expression cassette comprising a polynucleotide sequence comprising: a) a promoter sequence; b) a sequence encoding a polypeptide; and c) a ribonucleic acid (RNA) export signal, wherein the promoter sequence is operably linked to the sequence encoding the FANCA polypeptide (SEQ ID NO: 25), and optionally where a)-c) are present in the expression cassette in 5′ to 3′ order. In certain embodiments, the promoter is a phosphoglycerate kinase (PGK) promoter. In certain embodiments, the sequence encoding the polypeptide is codon-optimized. In some embodiments, the sequence encoding the polypeptide is a codon-optimized version of the human FANCA cDNA having at least 85% identity to SEQ ID NO: 8. In particular embodiments, the RNA export signal is a mutated post-transcriptional regulatory element of the woodchuck hepatitis virus (wPRE).

In certain embodiments, the mutated wPRE is a chimeric wPRE comprising a sequence having at least 80% identity to SEQ ID NO: 23. In some embodiments, the expression cassette further comprising one or more enhancer sequences. In some embodiments, the expression cassette further comprises a polypurine tract (PPT) or polyadenylation (polyA) signal sequence. In some embodiments, the expression cassette further comprises one or more of the following sequences: i) a packing signal sequence; ii) a truncated Gag sequence; iii) a Rev responsive element (RRE); iv) a central polypurine tract (cPPT); v) a central terminal sequence (CTS); and vi) an upstream sequence element (USE), optionally from simian virus 40 (SV40-USE). In some embodiments, the expression cassette further comprises 5′ and 3′ long terminal repeat (LTR) sequences.

In a related embodiment, the present invention provides a recombinant gene delivery vector comprising an expression cassette disclosed herein. In certain embodiments, the recombinant gene delivery vector is a virus or viral vector. In certain embodiments, the virus or viral vector is a lentivirus (LV).

In another related embodiment, the present invention provides a cell comprising an expression cassette or gene delivery vector disclosed herein. In some embodiments, the cell is a blood cell. In some embodiments, the cell is an erythroid cell. In some embodiments, the cell is a bone marrow cell, e.g., a lineage depleted bone marrow cell. In particular embodiments, the cell is a hematopoietic stem cell or a CD34⁺ cell. In some embodiments, the cell is a hematopoietic stem cell. In some embodiments, the cell is a CD34+ hematopoietic stem cell. In some embodiments, the cell is a committed hematopoietic erythroid progenitor cell.

In a related embodiment, the present invention provides a pharmaceutical composition comprising a pharmaceutically acceptable excipient and recombinant gene delivery vector or cell disclosed herein. In certain aspects of the invention, pharmaceutical compositions are provided comprising a polynucleotide cassette of the invention and a pharmaceutical excipient. In other embodiments, the pharmaceutical composition comprises a gene delivery vector of the invention and a pharmaceutical excipient.

Methods and compositions are provided for the use of gene therapy vector compositions, e.g., viral vectors, comprising these genetic expression cassettes for use in the preparation of medicaments useful in central and targeted gene therapy of diseases, disorders, and dysfunctions in an animal, and in humans in particular.

In another embodiment, the present invention provides a method of treating or preventing a disease or disorder in a subject in need thereof, comprising providing to the subject an expression cassette, gene delivery vector, or pharmaceutical composition disclosed herein.

In another embodiment, the present invention includes a method of treating Fanconi anemia in a subject in need thereof, comprising providing to the subject a pharmaceutical composition disclosed herein.

In a related embodiment, the present invention includes a method for treating Fanconi anemia in a subject in need thereof, comprising providing to the subject CD34⁺ cells comprising an expression cassette, wherein the expression cassette comprises a polynucleotide sequence comprising in the following 5′ to 3′ order: (a) a human phosphoglycerate kinase (PGK) promoter sequence or a functional homolog or variant thereof, (b) a sequence encoding a human FANCA polypeptide or a functional fragment or variant thereof, (c) a woodchuck hepatitis virus regulatory element (WPRE) RNA export signal sequence or a functional variant or fragment thereof, wherein the sequence encoding the human FANCA polypeptide or functional fragment or variant thereof is operably linked to the PGK promoter sequence. In certain embodiments, the CD34⁺ cells were obtained from the subject. In particular embodiments, the CD34+ cells were obtained from the subject after the subject was treated with a combination of: (i) G-CSF or Filgrastin; and (ii) Plerifaxor. In particular embodiments, the CD34⁺ cells were transduced with the recombinant gene delivery vector comprising the expression cassette. In one embodiment, the CD34⁺ cells were transduced by contacting the CD34⁺ cells with the recombinant gene delivery vector for about 24 hours.

In another embodiment, the present invention provides a method for treating Fanconi anemia in a subject in need thereof, comprising: (a) providing to the subject a combination of: (i) G-CSF or Filgrastin; and (ii) Plerifaxor to mobilize CD34+ cells within the subject; (b) obtaining a biological sample comprising CD34⁺ cells from the subject, wherein the biological sample is optionally peripheral blood or bone marrow; (c) preparing a cell population enriched for CD34+ cells from the biological sample; (d) transducing the cell population enriched for CD34+ cells with a recombinant gene deliver vector comprising an expression cassette comprising a polynucleotide sequence comprising in the following 5′ to 3′ order: (i) a promoter sequence or a functional homolog or variant thereof, and (ii) a sequence encoding a human FANCA polypeptide or a functional fragment or variant thereof, wherein the sequence encoding the human FANCA polypeptide or functional fragment or variant thereof is operably linked to the PGK promoter sequence, where the transducing comprises contacting the cell population enriched for CD34⁺ cells with the lentiviral vector for about 24 hours; and (e) providing the cell population transduced with the lentiviral vector resulting from step (d) to the subject. In certain embodiments, preparing the cell population comprises depleting erythrocytes and/or enriching for CD34⁺ cells by positive selection, negative selection, or a combination thereof. In particular embodiments, the method inhibits the development of, halts progression of, and/or reverses progression of a hematological manifestation of Fanconi anemia in the subject. In particular embodiments, the hematological manifestation of Fanconi anemia is selected from one or more of BMF, thrombocytopenia, leukopenia, pancytopenia, neutropenia, and anemia.

Other features and advantages of the invention will be apparent from and encompassed by the following detailed description and claims.

### BRIEF DESCRIPTION OF THE DRAWINGS

FIG. 1 is schematic diagram of an exemplary construct, PGK-FANCA.WPRE*LV.

FIG. 2A shows a schematic representation of LVs expressing FANCA under the control of different internal promoters. FIG. 2B shows a Western blot analysis of FANCA in FA-A cells transduced with vectors shown in panel A shows correction of the MMC-hypersensitivity of hematopoietic progenitors from FA-A mice subjected to gene therapy. FA-A bone marrow (BM) cells were transduced with PGK_FANCA-WPRE* or control SF1-EGFP LVs and transplanted into irradiated FA-A mice. At 7 months post-transplantation BM samples were harvested and cultured in methylcellulose in the presence of increasing concentrations of mitomycin C (MMC).

FIG. 3 presents data showing the functional analysis of lentiviral vectors expressing FANCA under the control of different internal promoters. FIG. 3A shows reversion of MMC sensitivity of FA-A lymphoblast cell line (LCL) cells transduced with LVs. Mean values of 3 different experiments are shown. FIG. 3B show restored formation of nuclear FANCD2 foci in FA-A LCLs transduced with vectors and exposed to mitomycin C (MMC).

FIG. 4 presents data showing in vivo efficacy and safety of FA gene therapy with the PGK-FANCA.Wpre* LV FIG. 4A shows the construct. FIG. 4B depicts the methodology whereby Bone marrow (BM) cells from FA-A mice were transduced with FANCA LV and then transplanted into irradiated FA-A recipient mice. FIG. 4C shows BM samples from transplanted FA-A mice were cultured in methylcellulose in the absence and the presence of MMC.

FIG. 5 shows proviral copy number in FANCA^{-/-} mice transplanted with syngeneic bone marrow cells previously transduced with lentiviral vectors carrying the therapeutic FANCA gene under the control of the PGK promoter. PB=peripheral blood; BM=bone marrow.

FIG. 6 presents data showing correction of the MMC-hypersensitivity of hematopoietic progenitors from FAA mice subjected to gene therapy with the medicinal product. FA-A bone marrow (BM) cells were transduced with PGK_FANCA-Wpre* or SF1-EGFP LVs and transplanted into irradiated FA-A mice. At seven (7) months post-transplantation BM samples were harvested and cultured in methylcellulose in the presence of increasing concentrations of MMC.

FIG. 7 depicts improved transduction efficacy of cryopreserved bone marrow progenitors from three Fanconi Anemia patients. Samples were subjected to standard transductions consisting in a single transduction cycle (16 h) after 2 h of static preloading (white bars; 1×S) or improved transduction consisting in three transduction cycles (2 h+2 h+12 h) with the lentiviral vectors (grey bars; 3×D).

FIG. 8 shows the relevance of the WPRE sequence on the functional properties of lentiviral vectors expressing FANCA under the control of the PGK promoter. FIG. 8A: Reversion of the (MMC) sensitivity of FA-A LCLs transduced with PGK-FANCA and PGK-FANCA-WPRE LVs. Mean values of 3 different experiments are shown. FIG. 8B: Reversion of MMC sensitivity of FA-A hematopoietic progenitors (colony forming cells, CFCs) transduced with SFFV-FANCA LV and PGK-FANCA LVs (“Expt 1”) and with PGK-FANCA and PGK-FANCA-WPRE LVs (“Expt 2”). White bars=no MMC; Black bars=10 nM MMC. MMC=mitomycin C

FIG. 9 shows efficacy of GALV-TR and VSV-G pseudo typed lentiviral vectors to transduce hematopoietic progenitors from the bone marrow of Fanconi anemia patients with EGFP-LVs.

FIG. 10 shows low in vitro transformation potential of lentiviral vectors harboring the hPGK promoter. FIG. 10A: depiction of the vectors. FIG. 10B: transformation capacity as measured in re-plating frequency over copy number.

FIG. 11 is a depiction of an illustrative hematopoietic stem cell (HSC) collection and gene therapy trial of FA-A patients.

FIG. 12 shows the hematological parameters of recruited patients in the study described in the Examples. FIG. 12A shows results for hemoglobin. FIG. 12B shows results for neutrophils. FIG. 12C shows results for platelets. FIG. 12D shows results for CD34+ cells.

FIG. 13 illustrates the Fancostem protocol phase II study aiming at the evaluation of the safety and efficacy of the mobilization and collection of CD34+ cells after treatment with Plerixafor (MOZOBIL) and Filgrastim (also known as G-CSF) (NEUPOGENE) in patients with Fanconi anemia. The number of patients is 10.

FIG. 14 shows G-CSF/Plerixafor-mediated mobilization of CD34+ cells in FA-A patients.

FIG. 15 shows G-CSF/Plerixafor-mediated mobilization of CFCs in FA-A patients.

FIG. 16 is a summary of the CD34+ cells collected in G-CSF/Plerixafor mobilized FA-A patients. FIG. 16A shows CD34+ cell collection in FANCOSTEM and FIG. 16B shows compared to previous studies.

FIG. 17 is a chart showing the comparison between predicted CD34+ cell numbers in bone marrow (BM) versus actual numbers in mobilized peripheral blood (mPB).

FIG. 18 is a chart of the collection and purification of mobilized peripheral blood (mPB) FA-A CD34+ cells.

FIG. 19 shows CD34 expression prior to and after immunoselection of mobilized peripheral blood (mPB) CD34+ cells from healthy donors (HD) and FA patients.

FIG. 20 shows patient FA 02005 fit the criteria for both FANCOSTEM and FANCOLEN studies. FIG. 20A shows cell counts; FIG. 20B shows hematopoietic stem cell (HSC) content versus age.

FIGS. 21A-21E present test results showing FA diagnosis of patient FA-02005 prior to gene therapy.

FIG. 22 shows the follow up parameter of the cell manufacturing process for FA-A Patient 02005.

FIG. 23 is a graph depicting vector copy number prior to and at 2 weeks, 4 weeks, 6 weeks, 2 months, 3 months, 4 months, and 5 months after gene therapy in patent FA-02005.

FIG. 24 presents follow-up of the first not-conditioned FA-A patient (FA-02005) prior to and after gene therapy as measured by hemoglobin amounts.

FIG. 25 presents follow-up of the first not-conditioned FA-A patient (FA-02005) prior to and after gene therapy as measured by neutrophil amounts.

FIG. 26 presents follow-up of the first not-conditioned FA-A patient (FA-02005) prior to and after gene therapy as measured by platelet amounts.

FIG. 27 is a chart of the hematological evolution of patient FA-A 02002.

FIG. 28 shows the diagnosis of FA 02002 as Not mosaic; Homozygote FANCA c.239 C>T p.Gln99*MMC Hypersensitive; Complemented by FANC.

FIG. 29 shows the cell manufacturing process in patient FA-A 02002.

FIGS. 30A-30E present the analysis of CD34 expression in a healthy donor (HD) and FA mobilized peripheral blood (mPB) during the different steps required for LV-transduction in patient FA 02002.

FIG. 31 is a graph depicting vector copy number prior to and after gene therapy in patient FA 02002.

FIG. 32 presents data of the follow up of patient FA-A 02002 infused with cryopreserved cells as measured by hemoglobin amount.

FIG. 33 presents data of the follow up of patient FA-A 02002 infused with cryopreserved cells as measured by neutrophil amount.

FIG. 34 presents data of the follow up of patient FA-A 02005 infused with cryopreserved cells as measured by platelet amount.

FIG. 35 shows transduction of fresh mobilized peripheral blood (mPB) CD34+ cells from FA-A patients using validated conditions. FIG. 35A presents the protocol. FIG. 35B shows a graph of results from patient 02002. FIG. 35C shows a graph of results from patient 02003. FIG. 35D shows a graph of results from patient 02004.

FIG. 36 shows data for the engraftment of corrected FA-A mPB CD34+ cells in NSG mice. FIG. 36A shows the protocol. FIG. 36B shows results for patient 02002. FIG. 36C shows results for patient 02003. FIG. 36D shows results for patient 02004. mPB=mobilized peripheral blood.

FIG. 37 depicts in vivo selection of corrected FA HPCs from patient 02002 in NOD scid gamma (NSG) mice. FIG. 37A shows the protocol. FIG. 37B is a graph of CFCs pre-transplantation. FIG. 37C is a graph of hCFCs 30 days post transplantation.

FIG. 38 is a map of the 5.7 kb plasmid encoding the envelope G glycoprotein of the VSV under the control of the CMV promoter and carries the kanamycin resistant gene for selection purposes.

FIG. 39 is a map of the 3.5 kb plasmid encoding for the HIV-1 rev gene under the control of the CMV promoter and carries the kanamycin resistance gene for selection purposes.

FIG. 40 is a map of the 8.8 kb plasmid containing the HIV-1 gag and pol genes that code for the HIV-1 structural and enzymatic proteins under the control of the CMV promoter. It contains intron 2 of the human beta globin (HBB2), the HIV-1 Rev responsive element (RRE) and the kanamycin resistance gene.

FIG. 41 is a map of the 11621 base pair transfer cassette pCCL-SIN-cPPT/CTS-hPGK-hFANCA-WPRE.

FIGS. 42A-42D represent the LAM-PCR analysis of FANCA-LV insertion sites in FA hematopoietic stem cells (HSC).

FIG. 43 depicts LAM-PCR results for tracking of FANCA-LV treated cells. FIG. 43A depicts the protocol and FIG. 43B is a chart of the data.

FIG. 44 shows the clonal diversity of Fanca−/− recipients transplanted with LV-corrected HSCs. FIG. 44A shows the protocol and FIG. 44B presents a graph of the data.

### DETAILED DESCRIPTION OF THE INVENTION

The present invention relates generally to the fields of molecular biology and virology, and in particular, to gene expression cassettes, and vectors comprising them useful for the delivery of nucleic acid segments encoding selected therapeutic constructs (including for example, peptides, polypeptides, ribozymes, and catalytic RNA molecules), to selected cells and tissues of vertebrate animals. In particular, these genetic constructs are useful in gene therapy for the treatment of mammalian, and in particular, human diseases, disorders, and dysfunctions related to FANCA gene product dysregulation.

In certain embodiments, the invention provides compositions and methods for gene therapy treatment of subjects with Fanconi Anemia (FA). In particular, compositions and methods for rescuing FANCA gene expression are provided. Specific methods disclosed herein relate to the use of lentiviral vectors to deliver human FANCA to hematopoietic progenitor cells of a subject with FA, particularly FA-A.

### Definitions

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

A “vector” as used herein refers to a macromolecule or association of macromolecules that comprises or associates with a polynucleotide and which can be used to mediate delivery of the polynucleotide to a cell. Illustrative vectors include, for example, plasmids, viral vectors (e.g., retroviral vectors, such as lentiviral vectors), liposomes, and other gene delivery vehicles.

The term “LV” is an abbreviation for lentivirus, and may be used to refer to the virus itself or derivatives thereof. The term covers all subtypes and both naturally occurring and recombinant forms, except where required otherwise.

As used herein, the term “gene” or “coding sequence” refers to a nucleotide sequence in vitro or in vivo that encodes a gene product. In some instances, the gene consists or consists essentially of coding sequence, that is, sequence that encodes the gene product. In other instances, the gene comprises additional, non-coding, sequence. For example, the gene may or may not include regions preceding and following the coding region, e.g., 5′ untranslated (5′ UTR) or “leader” sequences and 3′ UTR or “trailer” sequences, as well as intervening sequences (introns) between individual coding segments (exons).

As used herein, a “therapeutic gene” refers to a gene that, when expressed, confers a beneficial effect on the cell or tissue in which it is present, or on a mammal in which the gene is expressed. Examples of beneficial effects include amelioration of a sign or symptom of a condition or disease, prevention or inhibition of a condition or disease, or conferral of a desired characteristic. Therapeutic genes include genes that correct a genetic deficiency in a cell or mammal.

As used herein, a transgene is a gene that is delivered to a cell by a vector.

As used herein, the term “gene product” refers to the desired expression product of a polynucleotide sequence such as a polypeptide, peptide, protein or interfering RNA including short interfering RNA (siRNA), miRNA or small hairpin RNA (shRNA).

As used herein, the terms “polypeptide,” “peptide,” and “protein” refer to polymers of amino acids of any length. The terms also encompass an amino acid polymer that has been modified; for example, disulfide bond formation, glycosylation, lipidation, phosphorylation, or conjugation with a labeling component.

By “comprising” it is meant that the recited elements are required in, for example, the composition, method, kit, etc., but other elements may be included to form the, for example, composition, method, kit etc. within the scope of the claim. For example, an expression cassette “comprising” a gene encoding a therapeutic polypeptide operably linked to a promoter is an expression cassette that may include other elements in addition to the gene and promoter, e.g., poly-adenylation sequence, enhancer elements, other genes, linker domains, etc.

By “consisting essentially of”, it is meant a limitation of the scope of the, for example, composition, method, kit, etc., described to the specified materials or steps that do not materially affect the basic and novel characteristic(s) of the, for example, composition, method, kit, etc. For example, an expression cassette “consisting essentially of” a gene encoding a therapeutic polypeptide operably linked to a promoter and a polyadenylation sequence may include additional sequences, e.g., linker sequences, so long as they do not materially affect the transcription or translation of the gene. As another example, a variant, or mutant, polypeptide fragment “consisting essentially of” a recited sequence has the amino acid sequence of the recited sequence plus or minus about 10 amino acid residues at the boundaries of the sequence based upon the full length naïve polypeptide from which it was derived, e.g., 10, 9, 8, 7, 6, 5, 4, 3, 2 or 1 residue less than the recited bounding amino acid residue, or 1, 2, 3, 4, 5, 6, 7, 8, 9, or 10 residues more than the recited bounding amino acid residue.

By “consisting of”, it is meant the exclusion from the composition, method, or kit of any element, step, or ingredient not specified in the claim. For example, an expression cassette “consisting of” a gene encoding a therapeutic polypeptide operably linked to a promoter, and a post-transcriptional regulatory element consists only of the promoter, polynucleotide sequence encoding the therapeutic polypeptide, and post-transcriptional regulatory element. As another example, a polypeptide “consisting of” a recited sequence contains only the recited sequence.

An “expression vector” as used herein encompasses a vector, e.g., plasmid, minicircle, viral vector, liposome, and the like as discussed above or as known in the art, comprising a polynucleotide which encodes a gene product of interest, and is used for effecting the expression of a gene product in an intended target cell. An expression vector also comprises control elements operatively linked to the encoding region to facilitate expression of the gene product in the target. The combination of control elements, e.g., promoters, enhancers, UTRs, miRNA targeting sequences, etc., and a gene or genes to which they are operably linked for expression is sometimes referred to as an “expression cassette.” Many such control elements are known and available in the art or can be readily constructed from components that are available in the art.

A “promoter” as used herein encompasses a DNA sequence that directs the binding of RNA polymerase and thereby promotes RNA synthesis, i.e., a minimal sequence sufficient to direct transcription. Promoters and corresponding protein or polypeptide expression may be ubiquitous, meaning strongly active in a wide range of cells, tissues and species or cell-type specific, tissue-specific, or species specific. Promoters may be “constitutive,” meaning continually active, or “inducible,” meaning the promoter can be activated or deactivated by the presence or absence of biotic or abiotic factors. Also included in the nucleic acid constructs or vectors of the invention are enhancer sequences that may or may not be contiguous with the promoter sequence. Enhancer sequences influence promoter-dependent gene expression and may be located in the 5′ or 3′ regions of the native gene.

An “enhancer” as used herein encompasses a cis-acting element that stimulates or inhibits transcription of adjacent genes. An enhancer that inhibits transcription also is termed a “silencer”. Enhancers can function (i.e., can be associated with a coding sequence) in either orientation, over distances of up to several kilobase pairs (kb) from the coding sequence and from a position downstream of a transcribed region.

A “termination signal sequence” as used herein encompasses any genetic element that causes RNA polymerase to terminate transcription, such as for example a polyadenylation signal sequence.

As used herein, the terms “operatively linked” or “operably linked” refers to a juxtaposition of genetic elements, e.g., promoter, enhancer, termination signal sequence, polyadenylation sequence, etc., wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

As used herein, the term “heterologous” means derived from a genotypically distinct entity from that of the rest of the entity to which it is being compared. For example, a polynucleotide introduced by genetic engineering techniques into a plasmid or vector derived from a different species is a heterologous polynucleotide. As another example, a promoter removed from its native coding sequence and operatively linked to a coding sequence with which it is not naturally found linked is a heterologous promoter. Thus, for example, an LV vector that includes a heterologous nucleic acid encoding a heterologous gene product is an LV vector that includes a nucleic acid not normally included in a naturally-occurring, wild-type LV, and the encoded heterologous gene product is a gene product not normally encoded by a naturally-occurring, wild-type LV.

The term “endogenous” as used herein with reference to a nucleotide molecule or gene product refers to a nucleic acid sequence, e.g., gene or genetic element, or gene product, e.g., RNA, protein, that is naturally occurring in or associated with a host virus or cell.

The term “native” as used herein refers to a nucleotide sequence, e.g., gene, or gene product, e.g., RNA, protein, that is present in a wildtype virus or cell.

The term “variant” as used herein refers to a mutant of a reference polynucleotide or polypeptide sequence, for example a native polynucleotide or polypeptide sequence, i.e., having less than 100% sequence identity with the reference polynucleotide or polypeptide sequence. Put another way, a variant comprises at least one amino acid difference (e.g., amino acid substitution, amino acid insertion, amino acid deletion) relative to a reference polynucleotide sequence, e.g., a native polynucleotide or polypeptide sequence. For example, a variant may be a polynucleotide having a sequence identity of 70% or more with a full length native polynucleotide sequence, e.g., an identity of 75% or 80% or more, such as 85%, 90%, or 95% or more, for example, 98% or 99% identity with the full length native polynucleotide sequence. As another example, a variant may be a polypeptide having a sequence identity of 70% or more with a full length native polypeptide sequence, e.g., an identity of 75% or 80% or more, such as 85%, 90%, or 95% or more, for example, 98% or 99% identity with the full length native polypeptide sequence. Variants may also include variant fragments of a reference, e.g., native, sequence sharing a sequence identity of 70% or more with a fragment of the reference, e.g., native, sequence, e.g., an identity of 75% or 80% or more, such as 85%, 90%, or 95% or more, for example, 98% or 99% identity with the native sequence.

As used herein, the terms “biological activity” and “biologically active” refer to the activity attributed to a particular biological element in a cell. For example, the “biological activity” of an “immunoglobulin”, “antibody” or fragment or variant thereof refers to the ability to bind an antigenic determinant and thereby facilitate immunological function. As another example, the biological activity of a polypeptide or functional fragment or variant thereof refers to the ability of the polypeptide or functional fragment or variant thereof to carry out its native functions of, e.g., binding, enzymatic activity, etc. As a third example, the biological activity of a gene regulatory element, e.g., promoter, enhancer, kozak sequence, and the like, refers to the ability of the regulatory element or functional fragment or variant thereof to regulate, i.e., promote, enhance, or activate the translation of, respectively, the expression of the gene to which it is operably linked.

The terms “administering” or “introducing”, as used herein, refer to delivery of a vector for recombinant protein expression to a cell, to cells and/or organs of a subject, or to a subject. Such administering or introducing may take place in vivo, in vitro or ex vivo. A vector for expression of a gene product may be introduced into a cell by transfection, which typically means insertion of heterologous DNA into a cell by physical means (e.g., calcium phosphate transfection, electroporation, microinjection or lipofection); infection, which typically refers to introduction by way of an infectious agent, i.e., a virus; or transduction, which typically means stable infection of a cell with a virus or the transfer of genetic material from one microorganism to another by way of a viral agent (e.g., a bacteriophage).

“Transformation” is typically used to refer to bacteria comprising heterologous DNA or cells which express an oncogene and have therefore been converted into a continuous growth mode such as tumor cells. A vector used to “transform” a cell may be a plasmid, virus or other vehicle.

Typically, a cell is referred to as “transduced”, “infected”; “transfected” or “transformed” dependent on the means used for administration, introduction or insertion of heterologous DNA (i.e., the vector) into the cell. The terms “transduced”, “transfected” and “transformed” may be used interchangeably herein regardless of the method of introduction of heterologous DNA.

The term “host cell”, as used herein refers to a cell which has been transduced, infected, transfected or transformed with a vector. The vector may be a plasmid, a viral particle, a phage, etc. The culture conditions, such as temperature, pH and the like, are those previously used with the host cell selected for expression, and will be apparent to those skilled in the art. It will be appreciated that the term “host cell” refers to the original transduced, infected, transfected or transformed cell and progeny thereof.

The terms “treatment”, “treating” and the like are used herein to generally mean obtaining a desired pharmacologic and/or physiologic effect. The effect may be prophylactic in terms of completely or partially preventing a disease or symptom thereof, e.g., reducing the likelihood that the disease or symptom thereof occurs in the subject, and/or may be therapeutic in terms of a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting the disease, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “host,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.). The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The practice of the present invention employs, unless otherwise indicated, conventional techniques of cell biology, molecular biology (including recombinant techniques), microbiology, biochemistry and immunology, which are within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991), each of which is expressly incorporated by reference herein.

Several aspects of the invention are described below with reference to example applications for illustration. It should be understood that numerous specific details, relationships, and methods are set forth to provide a full understanding of the invention. One having ordinary skill in the relevant art, however, readily recognizes that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

Unless otherwise indicated, all terms used herein have the same meaning as they would to one skilled in the art and the practice of the present invention will employ conventional techniques of microbiology and recombinant DNA technology, which are within the knowledge of those of skill of the art.

In certain embodiments, the present disclosure provides polynucleotides, polynucleotide cassettes and expression vectors for the expression of a gene in cells. Also provided are pharmaceutical compositions and methods for the use of any of the compositions in promoting the expression of a gene in cells, for example, in an individual, e.g. for the treatment or prophylaxis of a disorder. These and other objects, advantages, and features of the invention will become apparent to those persons skilled in the art upon reading the details of the compositions and methods as more fully described below.

In certain embodiments, methods and compositions are provided for preparation of gene therapy vector compositions, e.g., viral vectors, comprising these genetic expression cassettes for use in the preparation of medicaments useful in central and targeted gene therapy of diseases, disorders, and dysfunctions in an animal, and in humans in particular.

In some embodiments, the present invention provides for gene therapy for Fanconi Anemia based on a LV vector harbouring the hPGK eukaryotic promoter that drives the expression of the FANCA cDNA. This therapeutic vector may be used to transduce human hematopoietic stem cells (HSCs), which may be subsequently transplanted into humans with Fanconi Anemia.

In certain embodiments, the present invention provides an FANCA LV vector for the genetic correction of Fanconi Anemia. Overall, results demonstrate feasibility of gene therapy for FA with a LV designed for clinical application.

The disclosed compositions may be utilized in a variety of investigative, diagnostic and therapeutic regimens, including the prevention and treatment of a variety of human diseases. The various compositions and methods of the invention are described below.

Although particular compositions and methods are exemplified herein, it is understood that any of a number of alternative compositions and methods are applicable and suitable for use in practicing the invention. It will also be understood that an evaluation of the expression constructs and methods of the invention may be carried out using procedures standard in the art.

### Fanconi Anemia

Fanconi Anemia (FA) is a rare inherited chromosomal instability syndrome mainly characterized by bone marrow failure (BMF) and cancer predisposition (Butturini A et al. Blood. 1994; 84:1650-1655; Kutler D I et al. Blood. 2003; 101:1249-1256). The prevalence of FA is 1-5 per million, and the heterozygote carrier frequency is estimated to be 1 in 300 (Tamary H et al. Eur J Haematol. 2004; 72:330-335).

FA is both genetically and phenotypically heterogeneous. To date, thirteen (13) complementation groups have been reported (FA-A, B, C, D1, D2, E, F, G, I, J, L, M and N) associated with mutations in the corresponding 13 Fanconia Anemia Complementation group (FANC) genes: FANCA, FANCB, FANCC, FANCD1/BRCA2, FANCD2, FANCE, FANCF, FANCG/XRCC9, FANCI, BRIP1/FANCJ, FANCL, FANCM/Hef and FANCN/PABLB2 (Wang W. Nat Rev Genet. 2007; 8:735-748). Except in the case of FA-B patients (FANCB is located in the X chromosome), FA is autosomal recessive.

Proteins encoded by FA genes participate in a biochemical route known as the FA/BRCA pathway (See Wang W. Nat Rev Genet. 2007; 8:735-748). Thirteen FA proteins have been identified in the FA pathway, each of them participating in one of the three FA protein complexes characterized so far in this pathway. The upstream complex, the FA core complex, is integrated by eight FA proteins (FANCA, FANCB, FANCC, FANCE, FANCF, FANCG, FANCL, FANCM) and two FA associated proteins (FAAP24 and FAAP100). A second complex is formed by FANCD2 and FANCI, which work together in the FA-ID complex. Due to the E3 ligase activity (FANCL) of the FA core complex, FANCD2 and FANCI can be mono-ubiquitinated and then loaded onto chromatin, forming large nuclear foci in response to DNA damage or replication arrest. Finally, mono-ubiquitinated FANCD2/FANCI interact with downstream FA proteins such as FANCJ/BRIP1, FANCN/PALB2 and FANCD1/BRCA2, which form stable complexes with proteins participating in homology directed repair (HDR), like BRCA1 and RAD51.

FA-A is the most frequent FA complementation group with about 50%-80% of FA patients corresponding to this complementation group (Casado J A et al. J Med Genet. 2007; 44:241-249; Levitus M et al. Blood. 2004; 103:2498-2503; Taniguchi T, D'andrea A D. Blood. 2006; 107:4223-4233). As a consequence of the deficient or null expression of FANCA, the FA core complex cannot be formed. This prevents the activation of the ID complex, and consequently the migration of these proteins to chromatin, thus resulting in the characteristic phenotype of FA cells.

As reviewed by D'Andrea et al. (Blood. 1997; 90:1725-1736), FA cells are characterized by different cellular phenotypes, mainly related to defects in cell survival, DNA repair and genomic stability.

FA is mainly characterized by congenital abnormalities, development of bone marrow failure, and a high risk of developing acute myeloid leukemia and certain solid tumors. On average, 70% of FA patients have congenital defects. The skeletal abnormalities (radial ray, hip, vertebral scoliosis, rib), and generalized skin hyperpigmentation, café au lait spots, are present in 60-70% of FA patients. Most patients have short stature, and around one-third of them have microphthalmia and renal abnormalities. In about 30% of FA patients, no obvious congenital abnormalities are observed (Tischkowitz M, Dokal I. Br J Haematol. 2004; 126:176-191).

The most important clinical features of FA patients are hematological. Bone marrow failure (BMF) is the main characteristic of the disease. It generally appears between the ages of 5 and 10 years. Eighty percent of 15 year-old patients develop BMF, with the actuarial risk of BMF above 90% by 40 years of age (Butturini et al., 1994, Kutler et al., 2003). Thrombocytopenia or leukopenia typically precedes anemia. Pancytopenia generally worsens over time. Neutropenia is associated with an increased risk for infections.

FA patients are also prone to develop cancer, principally, acute myeloid leukemia (AML) and myelodysplastic syndrome (MDS). The majority of tumors associated with FA develop after age 13 years, with an average age of 23 years. The relative risk for AML is increased 785-fold, with the median age of FA patients who develop AML being 14 years, and the cumulative incidence of hematological malignancy 30-55% by 40 years of age (Kutler et al., 2003; Rosenberg P S et al. Blood. 2003; 101:822-826). Older FA patients also have a high risk to develop solid tumors, mainly squamous cell carcinomas (SCC). The median age at which these patients develop solid tumors is 26 years, being the cumulative incidence of solid tumors 30% by the age of 40 (Kutler et al., 2003, Rosenberg et al., 2003).

Due to the complex clinical manifestations of FA, management of these patients is mainly focused on reducing symptoms of bone marrow failure (BMF), myeloid leukemia, and solid tumors. The limitations of each of the current therapies for patients with FA are reported in orphan medicinal products documentation for the lentiviral vector carrying the FANCA gene, whose sponsor is CIEMAT/CIBER on Rare Diseases (Bueren, J. (2010). Center for Biomedical Network Research on Rare Diseases Ref EU/3/10/822).

In certain embodiments, methods and compositions are provided for preparation of gene therapy vector compositions, e.g., viral vectors, comprising these genetic expression cassettes for use in the preparation of medicaments useful in central and targeted gene therapy of diseases, disorders, and dysfunctions in an animal, and in humans in particular.

In some embodiments, the present invention provides for gene therapy for Fanconi Anemia based on a LV vector harbouring the hPGK eukaryotic promoter that drives the expression of the FANCA cDNA. This therapeutic vector may be used to transduce human hematopoietic stem cells (HSCs), which may be subsequently transplanted into humans with Fanconi Anemia.

In certain embodiments, the present invention provides an FANCA LV vector for the genetic correction of Fanconi Anemia. Overall, results demonstrate feasibility of gene therapy for FA with a LV designed for clinical application.

The present disclosure includes gene expression cassettes (e.g., therapeutic cassettes), gene transfer cassettes comprising the gene expression cassettes (e.g., integration cassettes), plasmids comprising the gene transfer cassettes, and gene delivery vectors comprising the gene transfer cassettes. The gene expression cassettes, gene transfer cassettes, plasmids and gene delivery vectors comprising a polynucleotide sequence encoding a therapeutic gene product operably linked to a promoter sequence. In certain embodiments, the polynucleotide sequence is DNA or RNA. In certain embodiments, the gene expression cassette is a polynucleotide, the gene transfer cassette is a polynucleotide, and the vector is a virus, e.g., a lentivirus.

In certain embodiments, the therapeutic gene product is a FANCA protein or a functional fragment or variant thereof, optionally a wild-type human FANCA protein.

In particular embodiments, a gene expression cassette comprises a promoter region, a coding sequence, and a post-transcriptional regulatory element. In certain embodiments, the promoter region comprises a promoter sequence, or a functional fragment thereof. In one embodiment, the promoter is a human PGK promoter. In some embodiments, the expression cassette also comprises an RNA export signal. The RNA export signal may comprise a wPRE sequence. In some embodiments, a mutated wPRE, lacking any residual open reading frame (Schambach, Bohne et al. 2006) is included to improve the level of expression and stability of the therapeutic gene.

Some embodiments of the present invention comprise gene expression cassettes for the enhanced expression of a FANCA gene product. In some embodiments, the polynucleotide cassette comprises a wild type FANCA cDNA coding sequence, or a codon-optimized version of the human FANCA cDNA to increase mRNA stability upon transcription. For the optimization, GeneArt® software may be used, increasing the GC content and removing cryptic splice sites in order to avoid transcriptional silencing and therefore increase transgene expression. Alternatively, any optimization method known in the art may be used.

In some aspects of the invention, pharmaceutical compositions are provided comprising a gene delivery vector of the invention and a pharmaceutical excipient. In some embodiments, the pharmaceutical composition comprises a gene delivery vector of the invention and a pharmaceutical excipient.

In some aspects of the invention, methods are provided for expressing a transgene in mammalian cells. In some embodiments, the method comprises contacting one or more mammalian cells with an effective amount of a polynucleotide cassette of the invention or a gene delivery vector of the invention, wherein the transgene is expressed at detectable levels in the one or more mammalian cells. In some embodiments, the method comprises contacting one or more mammalian cells with an effective amount of a polynucleotide cassette of the invention or a gene delivery vector of the invention, wherein the transgene is expressed at therapeutic levels in the one or more mammalian cells. In some embodiments, the method is in vitro. In other embodiments, the method is in vivo.

In some aspects of the invention, methods are provided for the treatment or prophylaxis of a disease or disorder in a mammal in need of treatment or prophylaxis for a disease or disorder. In some embodiments, the method comprises administering to the mammal an effective amount of a pharmaceutical composition of the invention, wherein the coding sequence encodes a therapeutic gene product.

### Compositions

In some aspects of the disclosure, compositions are provided for the expression of a FANCA transgene in eukaryotic cells. In certain embodiments, the eukaryotic cell is a mammalian cell. In one embodiment, the mammalian cell is a hematopoietic stem cell (HSC). In one embodiment, the mammalian cell is a hematopoietic progenitor. In one embodiment, the mammalian cell is CD34+. In one embodiment, the mammalian cell is a human cell. In particular embodiments, the cell is a human CD34+ cell derived from a subject diagnosed with FA who is to be treated with a the CD34+ cell after it is transduced with a gene delivery disclosed herein, and comprises a gene expression cassette disclosed.

In one specific embodiment, the present disclosure includes a lentiviral vector comprising a gene expression cassette comprising a polynucleotide sequence encoding a therapeutic FANCA protein or a functional fragment or variant thereof. As used herein, a functional variant of a reference polynucleotide or polypeptide comprises one or more amino acid or nucleic acid deletions, additions or substitutions, as compared to the reference sequence, and it retains at least 50%, at least 80%, at least 90%, or at least 99% of the functional activity of the reference polynucleotide or polypeptide. As used herein, a functional fragment is a fragment of a reference polynucleotide or polypeptide, and it retains at least 50%, at least 80%, at least 90%, or at least 99% of the functional activity of the reference polynucleotide or polypeptide.

In one embodiment, the backbone of the lentiviral vector is the same as the one corresponding to the medicinal product “lentiviral vector carrying the Wiscott Aldrich Syndrome Protein (WASP-LV)” (Ref141/2000), although for FA treatment, the promoter is the human phosphoglycerate kinase (hPGK) promoter, characterized by its stable activity in vivo and by improved safety properties, compared to other promoters already used in gene therapy (Modlich U, Navarro S, Zychlinski D et al. Insertional Transformation of Hematopoietic Cells by Self-Inactivating Lentiviral And Gammaretroviral Vectors. Mol Ther. 2009; 17:1919-1928; Montini E, Cesana D, Schmidt M et al. Hematopoietic Stem Cell Gene Transfer in a Tumor prone Mouse Model Uncovers Low Genotoxicity Of Lentiviral Vector Integration. Nat Biotechnol. 2006; 24:687-696).

In some embodiments of the disclosure, the composition comprises a polynucleotide cassette. By a “polynucleotide cassette” is meant a polynucleotide sequence comprising two or more functional polynucleotide sequences, e.g., regulatory elements, translation initiation sequences, coding sequences, termination sequences, etc., typically in operable linkage to one another. Likewise, by a “polynucleotide cassette for the expression of a transgene in a mammalian cell,” it is meant a combination of two or more functional polynucleotide sequences, e.g., promoter, enhancer, 5′UTR, translation initiation sequence, coding sequence, termination sequences, etc. that promotes the expression of the transgene in a cell. Gene expression cassettes and gene transfer cassettes are examples of polynucleotide cassettes.

In some embodiments, the polynucleotide cassettes of the present disclosure provide for enhanced expression of a transgene in mammalian cells. As demonstrated by the working examples of the present disclosure, the present inventors have discovered a number of polynucleotide elements, i.e., improved elements as compared to those known in the art, which individually and synergistically provide for the enhanced expression of transgenes in mammalian cells. In certain embodiments, the arrangement of the two or more functional polynucleotide sequences within the polynucleotide cassettes of the present disclosure provide for enhanced expression of a transgene in mammalian cells. By “enhanced” it is meant that expression of the transgene is increased, augmented, or stronger, in cells carrying the polynucleotide cassettes of the present disclosure relative to in cells carrying the transgene operably linked to comparable regulatory elements, e.g., as known in the art. Put another way, expression of the transgene is increased, augmented, or stronger, from the polynucleotide cassettes of the present disclosure relative to expression from a polynucleotide cassette not comprising the one or more optimized elements of the present disclosure, i.e., a reference control. In certain embodiment, the enhanced expression is specific for or limited to one or more desired cell types.

For example, expression of the transgene may be enhanced, or augmented, or stronger, in cells comprising a polynucleotide cassette comprising a promoter disclosed herein than in cells that carry the transgene operably linked to a different promoter, e.g., as known in the art. As another example, expression of the transgene may be enhanced, or increased, augmented, or stronger, in cells comprising a polynucleotide cassette comprising an enhancer sequence disclosed herein than in cells that carry the transgene operably linked to a different enhancer sequence.

Without wishing to be bound by theory, enhanced expression of a transgene in cells is believed to be due to a faster build-up of gene product in the cells or a more stable gene product in the cells. Thus, enhanced expression of a transgene by the polynucleotide cassettes of the subject disclosure may be observed in a number of ways. For example, enhanced expression may be observed by detecting the expression of the transgene following contact of the polynucleotide cassette to the cells sooner, e.g. 2 days sooner, 7 days sooner, 2 weeks sooner, 3 weeks sooner, 4 weeks sooner, 8 weeks sooner, 12 weeks sooner or more, than expression would be detected if the transgene were operably linked to comparable regulatory elements, e.g., as known in the art. Enhanced expression may also be observed as an increase in the amount of gene product per cell. For example, there may be a 2-fold increase or more, e.g. a 3-fold increase or more, a 4-fold increase or more, a 5-fold increase or more, or a 10-fold increase or more in the amount of gene product per mammalian cell. Enhanced expression may also be observed as an increase in the number of mammalian cells that express detectable levels of the transgene carried by the polynucleotide cassette. For example, there may be a 2-fold increase or more, e.g. a 3-fold increase or more, a 4-fold increase or more, a 5-fold increase or more, or a 10-fold increase or more in the number of mammalian cells that express detectable levels of the transgene.

As another example, the polynucleotide of the present invention may promote detectable levels of the transgene in a greater percentage of cells as compared to a conventional polynucleotide cassette; for example, where a conventional cassette may promote detectable levels of transgene expression in, for example, less than 5% of the cells in a certain region, the polynucleotide of the present invention promotes detectable levels of expression in 5% or more of the cells in that region; e.g. 10% or more, 15% or more, 20% or more, 25% or more, 30% or more, 35% or more, 40% or more, or 45% or more, in some instances 50% or more, 55% or more; 60% or more, 65% or more, 70% or more, or 75% or more, for example 80% or more, 85% or more, 90% or more, or 95% or more of the cells that are contacted, will express detectable levels of gene product. Enhanced expression may also be observed as an alteration in the viability and/or function of the cells.

The polynucleotide cassettes of the present disclosure typically comprise a promoter region. Any suitable promoter region or promoter sequence therein can be used in the subject polynucleotide cassettes, so long as the promoter region promotes expression of a coding sequence in eukaryotic cells. In certain embodiments, the promoter region promotes expression of a coding sequence in mammalian cells. In some instances, the promoter is a ubiquitous promoter, i.e., it is a promoter that is active in a wide range of cells, tissues and species. In other instances, the promoter is a human PGK promoter.

Promoter and enhancer elements can be tissue specific or stage-specific. For example, a tissue-specific promoter or enhancer preferentially drives expression (or a higher level of expression) in one or more particular cell type. Examples of cell types include but are not limited to: hematopoietic stem cells, long term hematopoietic stem cells, short term hematopoietic stem cells, multipotent progenitors, hematopoietic CD34+ cells and any cluster differentiation subpopulation within the CD34+ population. A stage-specific promoter or enhancer preferentially drives expression (or higher level of expression) during one or more specific stages of the cell cycle or development. These include but are not limited to beta-globin locus control region, spectrin promoter, and an erythroid specific promoter.

In some embodiments, the polynucleotide comprises one or more enhancers. Enhancers are nucleic acid elements known in the art to enhance transcription, and can be located anywhere in association with the gene they regulate, e.g. upstream, downstream, within an intron, etc. Any enhancer element can be used in the polynucleotide cassettes and gene therapy vectors of the present disclosure, so long as it enhances expression of the gene when used in combination with the promoter.

The coding sequence to be expressed in the cells can be any polynucleotide sequence, e.g. gene or cDNA that encodes a gene product, e.g. a polypeptide or RNA-based therapeutic (siRNA, antisense, ribozyme, shRNA, etc.). The coding sequence may be heterologous to the promoter sequence to which it is operably linked, i.e. not naturally operably associated with it. Alternatively, the coding sequence may be endogenous to the promoter sequence to which it is operably linked, i.e. is associated in nature with that promoter. The gene product may act intrinsically in the mammalian cell, or it may act extrinsically, e.g., it may be secreted. For example, when the transgene is a therapeutic gene, the coding sequence may be any gene that encodes a desired gene product or functional fragment or variant thereof that can be used as a therapeutic for treating a disease or disorder. In various preferred embodiments, the transgene encodes human FANCA, i.e. SEQ ID NO: 25.

In one embodiment of the invention, the transgene coding sequence is modified, or “codon optimized” to enhance expression by replacing infrequently represented codons with more frequently represented codons. The coding sequence is the portion of the mRNA sequence that encodes the amino acids for translation. During translation, each of 61 trinucleotide codons are translated to one of 20 amino acids, leading to a degeneracy, or redundancy, in the genetic code. However, different cell types, and different animal species, utilize tRNAs (each bearing an anticodon) coding for the same amino acids at different frequencies. When a gene sequence contains codons that are infrequently represented by the corresponding tRNA, the ribosome translation machinery may slow, impeding efficient translation. Expression can be improved via “codon optimization” for a particular species, where the coding sequence is altered to encode the same protein sequence, but utilizing codons that are highly represented, and/or utilized by highly expressed human proteins (Cid-Arregui et al., 2003; J. Virol. 77: 4928). In one aspect of the present invention, the coding sequence of the transgene is modified to replace codons infrequently expressed in mammal or in primates with codons frequently expressed in primates. For example, in some embodiments, the coding sequence encoded by the transgene encodes a polypeptide having at least 85% sequence identity to a polypeptide encoded by a sequence disclosed above or herein, for example at least 90% sequence identity, e.g. at least 95% sequence identity, at least 98% identity, at least 99% identity, wherein at least one codon of the coding sequence has a higher tRNA frequency in humans than the corresponding codon in the sequence disclosed above or herein.

In an additional embodiment of the invention, the transgene coding sequence is modified to enhance expression by termination or removal of open reading frames (ORFs) that do not encode the desired transgene. An open reading frame (ORF) is the nucleic acid sequence that follows a start codon and does not contain a stop codon. ORFs may be in the forward or reverse orientation, and may be “in frame” or “out of frame” compared with the gene of interest. Such open reading frames have the potential to be expressed in an expression cassette alongside the gene of interest, and could lead to undesired adverse effects. In one aspect of the present invention, the coding sequence of the transgene has been modified to remove open reading frames by further altering codon usage. This was done by eliminating start codons (ATG) and introducing stop codons (TAG, TAA, or TGA) in reverse orientation or out-of-frame ORFs, while preserving the amino acid sequence and maintaining highly utilized codons in the gene of interest (i.e., avoiding codons with frequency <20%). In the present invention, the transgene coding sequence may be optimized by either of codon optimization and removal of non-transgene ORFs or using both techniques. As will be apparent to one of ordinary skill in the art, it is preferable to remove or minimize non-transgene ORFs after codon optimization in order to remove ORFs introduced during codon optimization.

In some embodiments, a polynucleotide cassette comprises:
(i) a phosphoglycerate kinase (PGK) promoter sequence or a functional variant or fragment thereof;
(ii) a sequence encoding a human FANCA protein or a functional fragment or variant thereof; and:
(iii) a post-transcriptional regulatory element of the woodchuck hepatitis virus (WPRE) sequence.

In some embodiments, a polynucleotide cassette comprises:
(i) a human phosphoglycerate kinase (PGK) promoter sequence;
(ii) a sequence encoding a human FANCA protein; and:
(iii) a mutant WPRE sequence.

In some embodiments, a polynucleotide cassette comprises:
a) a 5′ LTR, optionally a modified 5′ LTR;
b) a cPPT sequence;
c) PGK promoter sequence, optionally a human PGK promoter sequence;
d) a sequence encoding a human FANCA protein, optionally a cDNA sequence or a codon optimized sequence;
e) a mutant wPRE sequence; and
f) a 3′ LTR, optionally a modified 3′ LTR.

In one embodiment, the modified WPRE is referred to as WPRE*. WPRE* is a modified WPRE that lacks an open reading frame.

In certain embodiments, a gene transfer cassette comprises one or more additional elements, e.g., one or more elements selected from the following: 5′ LTR, 3′LTR, cPPT, CTS, RRE, enhancer sequences, and packaging signals.

The RRE sequence improves the efficiency of gene transfer. In particular embodiments of any of the expression cassettes and gene delivery vectors described herein, the RRE sequence comprises or consists of any of the following sequences, or sequences having at least 80%, at least 85%, at least 90%, at least 95%, at least 98%, or at least 99% identity to the following sequences:

(SEQ ID NO: 1)
(AGGAGCTTTGTTCCTTGGGTTCTTGGGAGCAGCAGGAAGCACTATGGGC
GCAGCGTCAATGACGCTGACGGTACAGGCCAGACAATTATTGTCTGGTAT
AGTGCAGCAGCAGAACAATTTGCTGAGGGCTATTGAGGCGCAACAGCATC
TGTTGCAACTCACAGTCTGGGGCATCAAGCAGCTCCAGGCAAGAATCCTG
GCTGTGGAAAGATACCTAAAGGATCAACAGCTCCT;
or

a sequence comprising or consisting of nucleotides 2649-2882 or SEQ ID NO:24.

The retroviral leader region contains the packaging signal (Ψ), which is involved in packaging the retroviral genome into the viral capsid. LV vectors were thought to require approximately 300 bp of the Gag gene in this region. Currently, this Gag sequence has been reduced to just 40 bp. In particular embodiments of any of the expression cassettes and gene delivery vectors described herein, the ψ sequence is an HIV-1 ψ sequence or the ψ sequence comprises or consists of any of the following sequences, or sequences having at least 80%, at least 85%, at least 90%, at least 95%, at least 98%, or at least 99% identity to the following sequences:

(SEQ ID NO: 2)
CTCTCTCGACGCAGGACTCGGCTTGCTGAAGCGCGCACGGCAAGAGGCGA
GGGGCGGCGACTGGTGAGTACGCCAAAAATTTTGACTAGCGGAGGCTAGA
AGGAGAGAGATGGGTGCGAGAGCGTC;
or

a sequence comprising or consisting of polynucleotides 2031-2156 of SEQ ID NO:24.

In particular embodiments of any of the expression cassettes and gene delivery vectors described herein, the truncated HIV-1 5′ LTR comprises or consists of any of the following sequences, or sequences having at least 80%, at least 85%, at least 90%, at least 95%, at least 98%, or at least 99% identity to any of the following sequences:

(SEQ ID NO: 3)
GGGTCTCTCTGGTTAGACCAGATCTGAGCCTGGGAGCTCTCTGGCTAACT
AGGGAACCCACTGCTTAAGCCTCAATAAAGCTTGCCTTGAGTGCTTCAAG
TAGTGTGTGCCCGTCTGTTGTGTGACTCTGGTAACTAGAGATCCCTCAGA
CCCTTTTAGTCAGTGTGGAAAATCTCTAGCA;
or

a sequence comprising or consisting of polynucleotides 1586-9495 of SEQ ID NO:24.

In particular embodiments of any of the expression cassettes and gene delivery vectors described herein, the HIV-1 self-inactivating 3′ LTR comprises or consists of any of the following sequences, or sequences having at least 80%, at least 85%, at least 90%, at least 95%, at least 98%, or at least 99% identity to the following sequences:

(SEQ ID NO: 4)
TGGAAGGGCTAATTCACTCCCAACGAAGACAAGATCTGCTTTTTGCTTGT
ACTGGGTCTCTCTGGTTAGACCAGATCTGAGCCTGGGAGCTCTCTGGCTA
ACTAGGGAACCCACTGCTTAAGCCTCAATAAAGCTTGCCTTGAGTGCTTC
AAGTAGTGTGTGCCCGTCTGTTGTGTGACTCTGGTAACTAGAGATCCCTC
AGACCCTTTTAGTCAGTGTGGAAAATCTCTAGCA;
or

a sequence comprising or consisting of polynucleotides 9262-9495 of SEQ ID NO:24.

In particular embodiments of any of the expression cassettes and gene delivery vectors described herein, the human cytomegalovirus (CMV) immediate early promoter comprises or consists of any of the following sequences, a functional fragment thereof, or a sequence having at least 80%, at least 85%, at least 90%, at least 95%, at least 98%, or at least 99% identity to any of the following sequences:

(SEQ ID NO: 5)
GTGATGCGGTTTTGGCAGTACATCAATGGGCGTGGATAGCGGTTTGACTC
ACGGGGATTTCCAAGTCTCCACCCCATTGACGTCAATGGGAGTTTGTTTT
GGCACCAAAATCAACGGGACTTTCCAAAATGTCGTAACAACTCCGCCCCA
TTGACGCAAATGGGCGGTAGGCGTGTACGGTGGGAGGTCTATATAAGCAG
AGCT;
or

a sequence comprising or consisting of polynucleotides 1586-1789 of SEQ ID NO:24.

The cPPT, which facilitates nuclear translocation of the pre-integration complexes, together with the CTS involved in the separation of reverse transcriptase, has been seen to improve viral titer (Zennou, et al. 2000; Follenzi et al. 2000). In particular embodiments of any of the expression cassettes and gene delivery vectors described herein, the central polypurine tract and central termination sequence of HIV-1 (cPPT/CTS) comprises or consists of any of the following sequences, a functional fragment thereof, or a sequence having at least 80%, at least 85%, at least 90%, at least 95%, at least 98%, or at least 99% identity to any of the following sequences:

(SEQ ID NO: 6)
TTTTAAAAGAAAAGGGGGGATTGGGGGGTACAGTGCAGGGGAAAGAATAG
TAGACATAATAGCAACAGACATACAAACTAAAGAATTACAAAAACAAATT
ACAAAAATTCAAAATTTT;
(SEQ ID NO: 12)
TTTAAAAGAAAAGGGGGGATTGGGGGGT;
or

a sequence comprising or consisting of nucleotides 3378-3495 of SEQ ID NO:24.

In particular embodiments of any of the expression cassettes and gene delivery vectors described herein, the human phosphoglycerate kinase 1 (hPGK) promoter comprises or consists of any of the following sequences, a functional fragment thereof, or a sequence having at least 80%, at least 85%, at least 90%, at least 95%, at least 98%, or at least 99% identity to any of the following sequences:

(SEQ ID NO: 7)
GGGGTTGGGGTTGCGCCTTTTCCAAGGCAGCCCTGGGTTTGCGCAGGGAC
GCGGCTGCTCTGGGCGTGGTTCCGGGAAACGCAGCGGCGCCGACCCTGGG
TCTCGCACATTCTTCACGTCCGTTCGCAGCGTCACCCGGATCTTCGCCGC
TACCCTTGTGGGCCCCCCGGCGACGCTTCCTGCTCCGCCCCTAAGTCGGG
AAGGTTCCTTGCGGTTCGCGGCGTGCCGGACGTGACAAACGGAAGCCGCA
CGTCTCACTAGTACCCTCGCAGACGGACAGCGCCAGGGAGCAATGGCAGC
GCGCCGACCGCGATGGGCTGTGGCCAATAGCGGCTGCTCAGCAGGGCGCG
CCGAGAGCAGCGGCCGGGAAGGGGCGGTGCGGGAGGCGGGGTGTGGGGCG
GTAGTGTGGGCCCTGTTCCTGCCCGCGCGGTGTTCCGCATTCTGCAAGCC
TCCGGAGCGCACGTCGGCAGTCGGCTCCCTCGTTGACCGAATCACCGACC
TCTCTCCCCAG;
or

a sequence comprising or consisting of nucleotides 3541-4051 of SEQ ID NO:24.

Since most FA patients belong to the FA-A complementation group (Casado et al., 2007, Levitus et al., 2004, Taniguchi et al., 2006), in particular embodiments, the encoded therapeutic gene product is FANCA, although the disclosure contemplates that FA proteins of other complementation groups may also be delivered, and thus encoded in the expression cassettes disclosed herein, e.g., instead of FANCA.

In particular embodiments of any of the expression cassettes and gene delivery vectors described herein, the polynucleotide sequence encoding FANCA is a human FANCA cDNA sequence that comprises or consists of the following sequence, or a functional fragment thereof, or a sequence having at least 80%, at least 85%, at least 90%, at least 95%, at least 98%, or at least 99% identity to the following sequence:

(SEQ ID NO: 8)
ATGTCCGACTCGTGGGTCCCGAACTCCGCCTCGGGCCAGGACCCAGGGGG
CCGCCGGAGGGCCTGGGCCGAGCTGCTGGCGGGAAGGGTCAAGAGGGAAA
AATATAATCCTGAAAGGGCACAGAAATTAAAGGAATCAGCTGTGCGCCTC
CTGCGAAGCCATCAGGACCTGAATGCCCTTTTGCTTGAGGTAGAAGGTCC
ACTGTGTAAAAAATTGTCTCTCAGCAAAGTGATTGACTGTGACAGTTCTG
AGGCCTATGCTAATCATTCTAGTTCATTTATAGGCTCTGCTTTGCAGGAT
CAAGCCTCAAGGCTGGGGGTTCCCGTGGGTATTCTCTCAGCCGGGATGGT
TGCCTCTAGCGTGGGACAGATCTGCACGGCTCCAGCGGAGACCAGTCACC
CTGTGCTGCTGACTGTGGAGCAGAGAAAGAAGCTGTCTTCCCTGTTAGAG
TTTGCTCAGTATTTATTGGCACACAGTATGTTCTCCCGTCTTTCCTTCTG
TCAAGAATTATGGAAAATACAGAGTTCTTTGTTGCTTGAAGCGGTGTGGC
ATCTTCACGTACAAGGCATTGTGAGCCTGCAAGAGCTGCTGGAAAGCCAT
CCCGACATGCATGCTGTGGGATCGTGGCTCTTCAGGAATCTGTGCTGCCT
TTGTGAACAGATGGAAGCATCCTGCCAGCATGCTGACGTCGCCAGGGCCA
TGCTTTCTGATTTTGTTCAAATGTTTGTTTTGAGGGGATTTCAGAAAAAC
TCAGATCTGAGAAGAACTGTGGAGCCTGAAAAAATGCCGCAGGTCACGGT
TGATGTACTGCAGAGAATGCTGATTTTTGCACTTGACGCTTTGGCTGCTG
GAGTACAGGAGGAGTCCTCCACTCACAAGATCGTGAGGTGCTGGTTCGGA
GTGTTCAGTGGACACACGCTTGGCAGTGTAATTTCCACAGATCCTCTGAA
GAGGTTCTTCAGTCATACCCTGACTCAGATACTCACTCACAGCCCTGTGC
TGAAAGCATCTGATGCTGTTCAGATGCAGAGAGAGTGGAGCTTTGCGCGG
ACACACCCTCTGCTCACCTCACTGTACCGCAGGCTCTTTGTGATGCTGAG
TGCAGAGGAGTTGGTTGGCCATTTGCAAGAAGTTCTGGAAACGCAGGAGG
TTCACTGGCAGAGAGTGCTCTCCTTTGTGTCTGCCCTGGTTGTCTGCTTT
CCAGAAGCGCAGCAGCTGCTTGAAGACTGGGTGGCGCGTTTGATGGCCCA
GGCATTCGAGAGCTGCCAGCTGGACAGCATGGTCACTGCGTTCCTGGTTG
TGCGCCAGGCAGCACTGGAGGGCCCCTCTGCGTTCCTGTCATATGCAGAC
TGGTTCAAGGCCTCCTTTGGGAGCACACGAGGCTACCATGGCTGCAGCAA
GAAGGCCCTGGTCTTCCTGTTTACGTTCTTGTCAGAACTCGTGCCTTTTG
AGTCTCCCCGGTACCTGCAGGTGCACATTCTCCACCCACCCCTGGTTCCC
AGCAAGTACCGCTCCCTCCTCACAGACTACATCTCATTGGCCAAGACACG
GCTGGCCGACCTCAAGGTTTCTATAGAAAACATGGGACTCTACGAGGATT
TGTCATCAGCTGGGGACATTACTGAGCCCCACAGCCAAGCTCTTCAGGAT
GTTGAAAAGGCCATCATGGTGTTTGAGCATACGGGGAACATCCCAGTCAC
CGTCATGGAGGCCAGCATATTCAGGAGGCCTTACTACGTGTCCCACTTCC
TCCCCGCCCTGCTCACACCTCGAGTGCTCCCCAAAGTCCCTGACTCCCGT
GTGGCGTTTATAGAGTCTCTGAAGAGAGCAGATAAAATCCCCCCATCTCT
GTACTCCACCTACTGCCAGGCCTGCTCTGCTGCTGAAGAGAAGCCAGAAG
ATGCAGCCCTGGGAGTGAGGGCAGAACCCAACTCTGCTGAGGAGCCCCTG
GGACAGCTCACAGCTGCACTGGGAGAGCTGAGAGCCTCCATGACAGACCC
CAGCCAGCGTGATGTTATATCGGCACAGGTGGCAGTGATTTCTGAAAGAC
TGAGGGCTGTCCTGGGCCACAATGAGGATGACAGCAGCGTTGAGATATCA
AAGATTCAGCTCAGCATCAACACGCCGAGACTGGAGCCACGGGAACACAT
TGCTGTGGACCTCCTGCTGACGTCTTTCTGTCAGAACCTGATGGCTGCCT
CCAGTGTCGCTCCCCCGGAGAGGCAGGGTCCCTGGGCTGCCCTCTTCGTG
AGGACCATGTGTGGACGTGTGCTCCCTGCAGTGCTCACCCGGCTCTGCCA
GCTGCTCCGTCACCAGGGCCCGAGCCTGAGTGCCCCACATGTGCTGGGGT
TGGCTGCCCTGGCCGTGCACCTGGGTGAGTCCAGGTCTGCGCTCCCAGAG
GTGGATGTGGGTCCTCCTGCACCTGGTGCTGGCCTTCCTGTCCCTGCGCT
CTTTGACAGCCTCCTGACCTGTAGGACGAGGGATTCCTTGTTCTTCTGCC
TGAAATTTTGTACAGCAGCAATTTCTTACTCTCTCTGCAAGTTTTCTTCC
CAGTCACGAGATACTTTGTGCAGCTGCTTATCTCCAGGCCTTATTAAAAA
GTTTCAGTTCCTCATGTTCAGATTGTTCTCAGAGGCCCGACAGCCTCTTT
CTGAGGAGGACGTAGCCAGCCTTTCCTGGAGACCCTTGCACCTTCCTTCT
GCAGACTGGCAGAGAGCTGCCCTCTCTCTCTGGACACACAGAACCTTCCG
AGAGGTGTTGAAAGAGGAAGATGTTCACTTAACTTACCAAGACTGGTTAC
ACCTGGAGCTGGAAATTCAACCTGAAGCTGATGCTCTTTCAGATACTGAA
CGGCAGGACTTCCACCAGTGGGCGATCCATGAGCACTTTCTCCCTGAGTC
CTCGGCTTCAGGGGGCTGTGACGGAGACCTGCAGGCTGCGTGTACCATTC
TTGTCAACGCACTGATGGATTTCCACCAAAGCTCAAGGAGTTATGACCAC
TCAGAAAATTCTGATTTGGTCTTTGGTGGCCGCACAGGAAATGAGGATAT
TATTTCCAGATTGCAGGAGATGGTAGCTGACCTGGAGCTGCAGCAAGACC
TCATAGTGCCTCTCGGCCACACCCCTTCCCAGGAGCACTTCCTCTTTGAG
ATTTTCCGCAGACGGCTCCAGGCTCTGACAAGCGGGTGGAGCGTGGCTGC
CAGCCTTCAGAGACAGAGGGAGCTGCTAATGTACAAACGGATCCTCCTCC
GCCTGCCTTCGTCTGTCCTCTGCGGCAGCAGCTTCCAGGCAGAACAGCCC
ATCACTGCCAGATGCGAGCAGTTCTTCCACTTGGTCAACTCTGAGATGAG
AAACTTCTGCTCCCACGGAGGTGCCCTGACACAGGACATCACTGCCCACT
TCTTCAGGGGCCTCCTGAACGCCTGTCTGCGGAGCAGAGACCCCTCCCTG
ATGGTCGACTTCATACTGGCCAAGTGCCAGACGAAATGCCCCTTAATTTT
GACCTCTGCTCTGGTGTGGTGGCCGAGCCTGGAGCCTGTGCTGCTCTGCC
GGTGGAGGAGACACTGCCAGAGCCCGCTGCCCCGGGAACTGCAGAAGCTA
CAAGAAGGCCGGCAGTTTGCCAGCGATTTCCTCTCCCCTGAGGCTGCCTC
CCCAGCACCCAACCCGGACTGGCTCTCAGCTGCTGCACTGCACTTTGCGA
TTCAACAAGTCAGGGAAGAAAACATCAGGAAGCAGCTAAAGAAGCTGGAC
TGCGAGAGAGAGGAGCTATTGGTTTTCCTTTTCTTCTTCTCCTTGATGGG
CCTGCTGTCGTCACATCTGACCTCAAATAGCACCACAGACCTGCCAAAGG
CTTTCCACGTTTGTGCAGCAATCCTCGAGTGTTTAGAGAAGAGGAAGATA
TCCTGGCTGGCACTCTTTCAGTTGACAGAGAGTGACCTCAGGCTGGGGCG
GCTCCTCCTCCGTGTGGCCCCGGATCAGCACACCAGGCTGCTGCCTTTCG
CTTTTTACAGTCTTCTCTCCTACTTCCATGAAGACGCGGCCATCAGGGAA
GAGGCCTTCCTGCATGTTGCTGTGGACATGTACTTGAAGCTGGTCCAGCT
CTTCGTGGCTGGGGATACAAGCACAGTTTCACCTCCAGCTGGCAGGAGCC
TGGAGCTCAAGGGTCAGGGCAACCCCGTGGAACTGATAACAAAAGCTCGT
CTTTTTCTGCTGCAGTTAATACCTCGGTGCCCGAAAAAGAGCTTCTCACA
CGTGGCAGAGCTGCTGGCTGATCGTGGGGACTGCGACCCAGAGGTGAGCG
CCGCCCTCCAGAGCAGACAGCAGGCTGCCCCTGACGCTGACCTGTCCCAG
GAGCCTCATCTCTTCTGA.

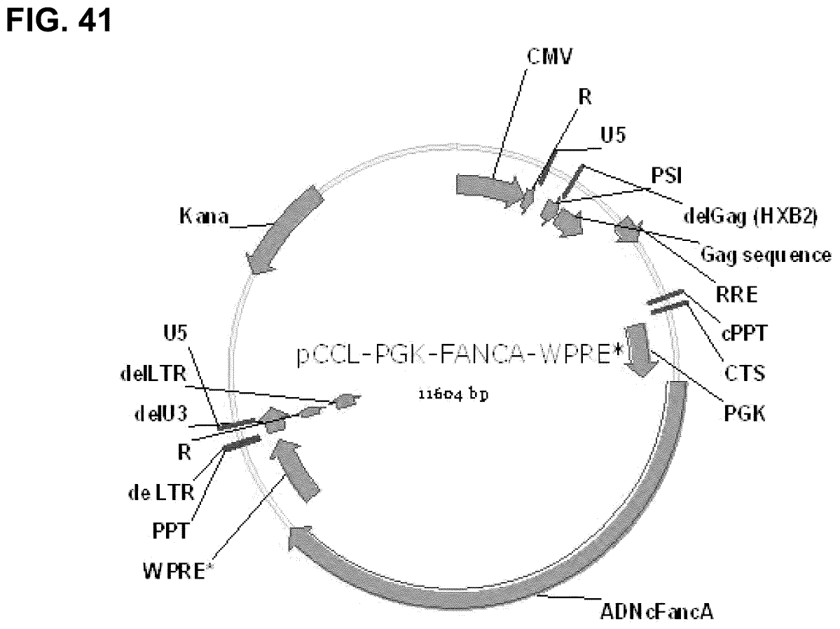

The present disclosure includes plasmids comprising an expression cassette or transfer cassette described herein. In particular embodiments, the plasmid is pCCL-PGK-FANCA-WPRE* (FIG. 41; SEQ ID NO: 24).

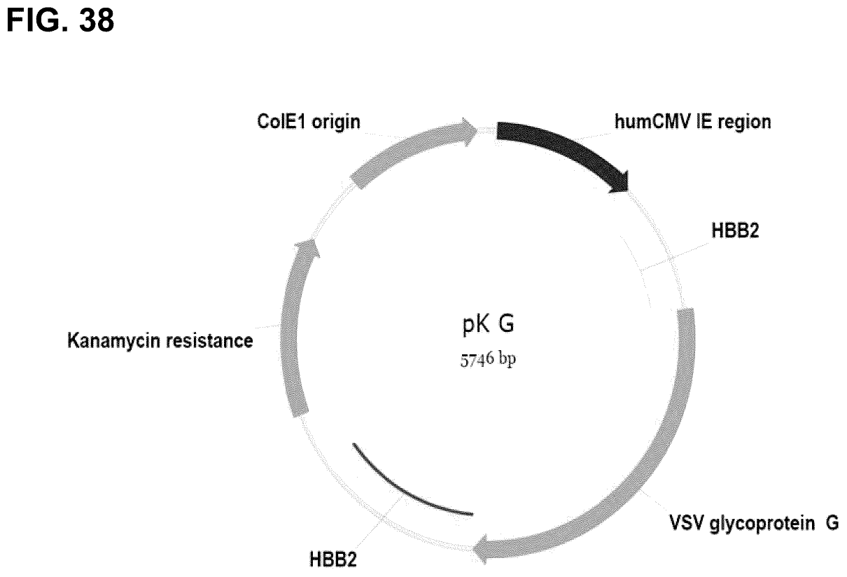

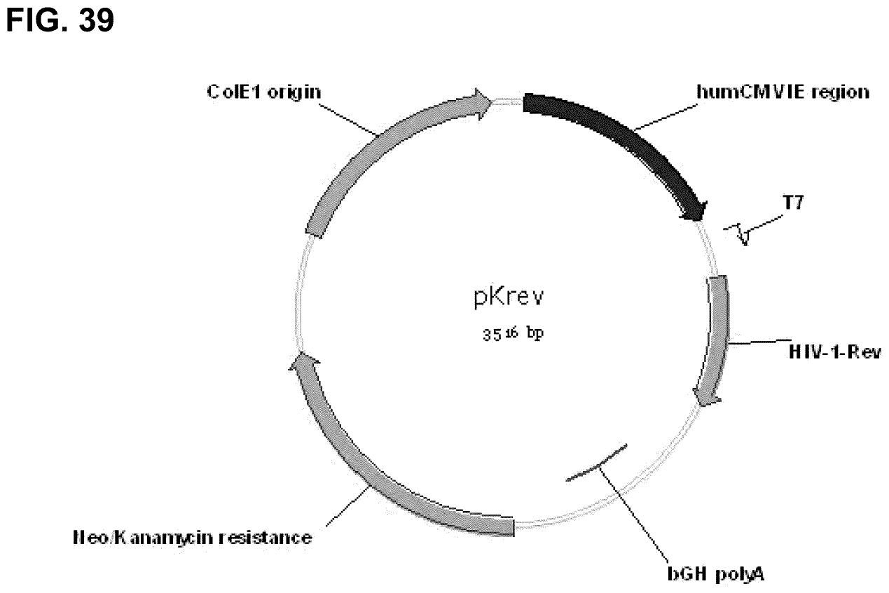

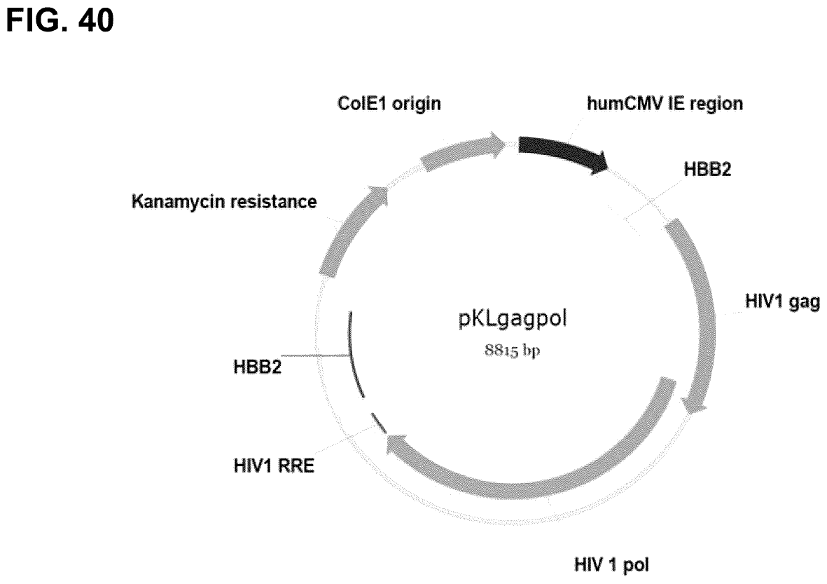

In certain embodiments, the disclosure includes a cell, e.g., a packaging cell or packaging cells line, e.g., 293 cells, comprising a plasmid disclosed herein. In particular embodiments, the cell comprises the plasmids depicted in FIGS. 38-41.

In certain embodiments, a transfer cassette or plasmid disclosed herein further comprises one or more additional elements, e.g., a CMV promoter and/or enhancer, an SV40 polyA sequence, an origin of replication, e.g., an SV40 on sequence, or any of the elements disclosed herein.

In particular embodiments of any of the transfer cassettes, plasmids or vectors described herein, the human CMV enhancer comprises or consists of the following sequence, a functional fragment thereof, or a sequence having at least 80%, at least 85%, at least 90%, at least 95%, at least 98%, or at least 99% identity to the following sequence:

(SEQ ID NO: 9)
GACATTGATTATTGACTAGTTATTAATAGTAATCAATTACGGGGTCATTA
GTTCATAGCCCATATATGGAGTTCCGCGTTACATAACTTACGGTAAATGG
CCCGCCTGGCTGACCGCCCAACGACCCCCGCCCATTGACGTCAATAATGA
CGTATGTTCCCATAGTAACGCCAATAGGGACTTTCCATTGACGTCAATGG
GTGGAGTATTTACGGTAAACTGCCCACTTGGCAGTACATCAAGTGTATCA
TATGCCAAGTACGCCCCCTATTGACGTCAATGACGGTAAATGGCCCGCCT
GGCATTATGCCCAGTACATGACCTTATGGGACTTTCCTACTTGGCAGTAC
ATCTACGTATTAGTCATCGCTATTACCATG.

In particular embodiments of any of the transfer cassettes, plasmids or vectors described herein described herein, the simian virus 40 (SV40) poly(A) signal comprises or consists of the following sequence, a functional fragment thereof, or a sequence having at least 80%, at least 85%, at least 90%, at least 95%, at least 98%, or at least 99% identity to the following sequence:

(SEQ ID NO: 10)
AACTTGTTTATTGCAGCTTATAATGGTTACAAATAAAGCAATAGCATCAC
AAATTTCACAAATAAAGCATTTTTTTCACTGCATTCTAGTTGTGGTTTGT
CCAAACTCATCAATGTATCTTA.

In particular embodiments of any of the transfer cassettes, plasmids or vectors described herein described herein, the SV40 origin of replication comprises or consists of the following sequence, a functional fragment thereof, or a sequence having at least 80%, at least 85%, at least 90%, at least 95%, at least 98%, or at least 99% identity to the following sequence:

(SEQ ID NO: 11)
ATCCCGCCCCTAACTCCGCCCAGTTCCGCCCATTCTCCGCCCCATGGCTG
ACTAATTTTTTTTATTTATGCAGAGGCCGAGGCCGCCTCGGCCTCTGAGC
TATTCCAGAAGTAGTGAGGAGGCTTTTTTGGAGGCC.

In some embodiments of any of the transfer cassettes, plasmids or vectors described herein described herein, the dNEF signal present in any of the expression cassettes or gene delivery vectors described herein comprises or consists of the following sequence, a functional fragment thereof, or a sequence having at least 80%, at least 85%, at least 90%, at least 95%, at least 98%, or at least 99% identity to the following sequence:

(SEQ ID NO: 13)
GAATTCGAGCTCGGTACCTTTAAGACCAATGACTTACAAGGCAGCTGTAG
ATCTTAGCCACTTTTTAAAAGAAAAGGGGGGAC.

In particular embodiments of any of the transfer cassettes, plasmids or vectors described herein described herein, the KanR sequence present in any of the expression cassettes or gene delivery vectors described herein comprises or consists of the following sequence:

(SEQ ID NO: 14)
ATGATTGAACAAGATGGATTGCACGCAGGTTCTCCGGCGGCTTGGGTGGA
GAGGCTATTCGGCTATGACTGGGCACAACAGACAATCGGCTGCTCTGATG
CCGCCGTGTTCCGGCTGTCAGCGCAGGGGCGTCCGGTTCTTTTTGTCAAG
ACCGACCTGTCCGGTGCCCTGAATGAACTGCAAGACGAGGCAGCGCGGCT
ATCGTGGCTGGCGACGACGGGCGTTCCTTGCGCGGCTGTGCTCGACGTTG
TCACTGAAGCGGGAAGGGACTGGCTGCTATTGGGCGAAGTGCCGGGGCAG
GATCTCCTGTCATCTCACCTTGCTCCTGCCGAGAAAGTATCCATCATGGC
TGATGCAATGCGGCGGCTGCATACGCTTGATCCGGCTACCTGCCCATTCG
ACCACCAAGCGAAACATCGCATCGAGCGAGCACGTACTCGGATGGAAGCC
GGTCTTGTCGATCAGGATGATCTGGACGAAGAGCATCAGGGGCTCGCGCC
AGCCGAACTGTTCGCCAGGCTCAAGGCGTCTATGCCCGACGGCGAGGATC
TCGTCGTGACCCACGGCGATGCCTGCTTGCCGAATATCATGGTGGAAAAT
GGCCGCTTTTCTGGATTCATCGACTGTGGCCGTCTGGGTGTGGCGGACCG
CTATCAGGACATAGCGTTGGCTACCCGTGATATTGCTGAAGAGCTTGGCG
GCGAATGGGCTGACCGCTTCCTTGTGCTTTACGGTATCGCCGCGCCCGAT
TCGCAGCGCATCGCCTTCTATCGCCTTCTTGACGAGTTCTTCTGA

In some embodiments of any of the transfer cassettes, plasmids or vectors described herein described herein, the rrnG terminator (transcription terminator from the E. coli ribosomal RNA rrnG operon (Albrechtsen et al., 1991) present in any of the expression cassettes or gene delivery vectors described herein comprises or consists of the following sequence:

(SEQ ID NO: 15)
GCATTGGCGCAGAAAAAAATGCCTGATGCGACGCTGCGCGTCTTATACTC
CCACATATGCCAGATTCAGCAACGGATACGGCTTCCCCAACTTGCCCACT
TCCATACGTGTCCTCCTTACCAGAAATTTATCCTTAA

In some embodiments of any of the transfer cassettes, plasmids or vectors described herein described herein, the ori (high-copy-number ColE1/pMB1/pBR322/pUC origin of replication) present in any of the expression cassettes or gene delivery vectors described herein comprises or consists of the following sequence, a functional fragment thereof, or a sequence having at least 80%, at least 85%, at least 90%, at least 95%, at least 98%, or at least 99% identity to the following sequence:

(SEQ ID NO: 16)
TTGAGATCCTTTTTTTCTGCGCGTAATCTGCTGCTTGCAAACAAAAAAAC
CACCGCTACCAGCGGTGGTTTGTTTGCCGGATCAAGAGCTACCAACTCTT
TTTCCGAAGGTAACTGGCTTCAGCAGAGCGCAGATACCAAATACTGTTCT
TCTAGTGTAGCCGTAGTTAGGCCACCACTTCAAGAACTCTGTAGCACCGC
CTACATACCTCGCTCTGCTAATCCTGTTACCAGTGGCTGCTGCCAGTGGC
GATAAGTCGTGTCTTACCGGGTTGGACTCAAGACGATAGTTACCGGATAA
GGCGCAGCGGTCGGGCTGAACGGGGGGTTCGTGCACACAGCCCAGCTTGG
AGCGAACGACCTACACCGAACTGAGATACCTACAGCGTGAGCTATGAGAA
AGCGCCACGCTTCCCGAAGGGAGAAAGGCGGACAGGTATCCGGTAAGCGG
CAGGGTCGGAACAGGAGAGCGCACGAGGGAGCTTCCAGGGGGAAACGCCT
GGTATCTTTATAGTCCTGTCGGGTTTCGCCACCTCTGACTTGAGCGTCGA
TTTTTGTGATGCTCGTCAGGGGGGCGGAGCCTATGGAAA.

In some embodiments of any of the transfer cassettes, plasmids or vectors described herein described herein, the CAP binding site present in any of the expression cassettes or gene delivery vectors described herein comprises or consists of the following sequence, a functional fragment thereof, or a sequence having at least 80%, at least 85%, at least 90%, at least 95%, at least 98%, or at least 99% identity to the following sequence:

(SEQ ID NO: 17)
TAATGTGAGTTAGCTCACTCAT.

In some embodiments of any of the transfer cassettes, plasmids or vectors described herein described herein, the E. coli lac promoter present in any of the expression cassettes or gene delivery vectors described herein comprises or consists of the following sequence, a functional fragment thereof, or a sequence having at least 80%, at least 85%, at least 90%, at least 95%, at least 98%, or at least 99% identity to the following sequence:

(SEQ ID NO: 18)
TTTACACTTTATGCTTCCGGCTCGTATGTTG.

In some embodiments of any of the transfer cassettes, plasmids or vectors described herein described herein, the lac operator present in any of the expression cassettes or gene delivery vectors described herein comprises or consists of the following sequence, a functional fragment thereof, or a sequence having at least 80%, at least 85%, at least 90%, at least 95%, at least 98%, or at least 99% identity to the following sequence:

(SEQ ID NO: 19)
TTGTGAGCGGATAACAA

In some embodiments of any of the transfer cassettes, plasmids or vectors described herein described herein, the T3 promoter (promoter for bacteriophage T3 RNA polymerase) present in any of the expression cassettes or gene delivery vectors described herein comprises or consists of the following sequence, a functional fragment thereof, or a sequence having at least 80%, at least 85%, at least 90%, at least 95%, at least 98%, or at least 99% identity to the following sequence: AATTAACCCTCACTAAAGG (SEQ ID NO: 20).

In some embodiments of any of the transfer cassettes, plasmids or vectors described herein described herein, the T7 promoter (promoter for bacteriophage T7 RNA polymerase) present in any of the expression cassettes or gene delivery vectors described herein comprises or consists of the following sequence, a functional fragment thereof, or a sequence having at least 80%, at least 85%, at least 90%, at least 95%, at least 98%, or at least 99% identity to the following sequence: CCTATAGTGAGTCGTATTA (SEQ ID NO: 21).

In some embodiments of any of the transfer cassettes, plasmids or vectors described herein described herein, the f1 on (f1 bacteriophage origin of replication) present in any of the expression cassettes or gene delivery vectors described herein comprises or consists of the following sequence, a functional fragment thereof, or a sequence having at least 80%, at least 85%, at least 90%, at least 95%, at least 98%, or at least 99% identity to the following sequence:

(SEQ ID NO: 22)
ACGCGCCCTGTAGCGGCGCATTAAGCGCGGCGGGTGTGGTGGTTACGCGC
AGCGTGACCGCTACACTTGCCAGCGCCCTAGCGCCCGCTCCTTTCGCTTT
CTTCCCTTCCTTTCTCGCCACGTTCGCCGGCTTTCCCCGTCAAGCTCTAA
ATCGGGGGCTCCCTTTAGGGTTCCGATTTAGTGCTTTACGGCACCTCGAC
CCCAAAAAACTTGATTAGGGTGATGGTTCACGTAGTGGGCCATCGCCCTG
ATAGACGGTTTTTCGCCCTTTGACGTTGGAGTCCACGTTCTTTAATAGTG
GACTCTTGTTCCAAACTGGAACAACACTCAACCCTATCTCGGTCTATTCT
TTTGATTTATAAGGGATTTTGCCGATTTCGGCCTATTGGTTAAAAAATGA
GCTGATTTAACAAAAATTTAACGCGAATT.

As discussed herein, the polynucleotide cassettes of the present invention may comprise an RNA export signal. Exemplary RNA export sequences include but are not limited to wPRE. The wPRE significantly increases transgene expression in target cells, by increasing RNA stability in a transgene, promoter and vector-independent manner (Zuffrey et al, 1999). However, it can express a truncated 60-amino acid protein derived from the WHV X gene involved in liver cancer (Kingsman et al, 2005). Therefore, most pre-clinical protocols and clinical trials include a mutated version of the wPRE element (Zanta-Boussif et al, 2009). On the other hand, the use of two SV40-USE elements in SIN-LV vectors has been seen to be more efficient than the wPRE sequence in suppressing transcriptional read through (Schambach et al, 2007). More precisely, the wPRE disclosed herein is a chimeric wPRE that carries 589 nucleotides from the modified WPRE performed by Axel Schambach (nucleotides 1-589) (WO 2008136670 A2; [5]) and 88 from a former wPRE (nucleotide 590-677) (Zuffrey et al, 1999). Data disclosed herein shows this chimeric wPRE works better than the former wPRE. The chimeric wPRE sequence comprises the following sequence, a functional fragment thereof, or a sequence having at least 80%, at least 85%, at least 90%, at least 95%, at least 98%, or at least 99% identity to the following sequence:

(SEQ ID NO: 23)
CGAGCATCTTACCGCCATTTATTCCCATATTTGTTCTGTTTTTCTTGATT
TGGGTATACATTTAAATGTTAATAAAACAAAATGGTGGGGCAATCATTTA
CATTTTTAGGGATATGTAATTACTAGTTCAGGTGTATTGCCACAAGACAA
ACATGTTAAGAAACTTTCCCGTTATTTACGCTCTGTTCCTGTTAATCAAC
CTCTGGATTACAAAATTTGTGAAAGATTGACTGATATTCTTAACTATGTT
GCTCCTTTTACGCTGTGTGGATATGCTGCTTTAATGCCTCTGTATCATGC
TATTGCTTCCCGTACGGCTTTCGTTTTCTCCTCCTTGTATAAATCCTGGT
TGCTGTCTCTTTATGAGGAGTTGTGGCCCGTTGTCCGTCAACGTGGCGTG
GTGTGCTCTGTGTTTGCTGACGCAACCCCCACTGGCTGGGGCATTGCCAC
CACCTGTCAACTCCTTTCTGGGACTTTCGCTTTCCCCCTCCCGATCGCCA
CGGCAGAACTCATCGCCGCCTGCCTTGCCCGCTGCTGGACAGGGGCTAGG
TTGCTGGGCACTGATAATTCCGTGGTGTTGTCGGGGAAGGGCCTGCTGCC
GGCTCTGCGGCCTCTTCCGCGTCTTCGCCTTCGCCCTCAGACGAGTCGGA
TCTCCCTTTGGGCCGCCTCCCCGCCTG.

In particular embodiments, the mutated WPRE sequence comprises or consists of WPRE*, which corresponds to nucleotides 8502-9178 of SEQ ID NO:24, or has at least 80%, at least 85%, at least 90%, at least 95%, at least 98%, or at least 99% identity to this region of SEQ ID NO:24.

Other combinations of elements both as disclosed herein or as known in the art will be readily appreciated by the ordinarily skilled artisan.

Additionally, as will be recognized by one of ordinary skill in the art, the polynucleotide cassettes may optionally contain other elements including, but not limited to restriction sites to facilitate cloning and regulatory elements for a particular gene expression vector.

In some aspects of the present invention, the subject polynucleotide cassettes are used to deliver a gene to cells, e.g. to determine the effect that the gene has on cell viability and/or function, to treat a cell disorder, etc. In various embodiments, delivery of a viral vector to cells by transduction may occur in vitro, ex vivo, or in vitro. Accordingly, in some aspects of the invention, the composition that provides for the expression of a transgene in mammalian cells is a gene delivery vector, wherein the gene delivery vector comprises a polynucleotide cassette, e.g., a gene transfer cassette, of the present disclosure.

Any convenient gene delivery vector that finds use delivering polynucleotide sequences to mammalian cells is encompassed by the gene delivery vectors of the present disclosure. For example, the vector may comprise single or double stranded nucleic acid, e.g. single stranded or double stranded DNA. For example, the gene delivery vector may be DNA, e.g., a naked DNA, e.g., a plasmid, a minicircle, etc. The vector may comprise single-stranded or double-stranded RNA, including modified forms of RNA. In another example, the gene delivery vector may be an RNA, e.g., an mRNA or modified mRNA.

As another example, the gene delivery vector may be a viral vector derived from a virus, e.g., an adenovirus, an adeno-associated virus, a lentivirus (LV), a herpes virus, an alphavirus or a retrovirus, e.g., Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) or Rous Sarcoma Virus (RSV). While embodiments encompassing the use of LV are described in greater detail below, it is expected that the ordinarily skilled artisan will appreciate that similar knowledge and skill in the art can be brought to bear on non-LV gene therapy vectors as well.

In some embodiments, the gene delivery vector is a self-limiting LV. In a specific embodiment of any of the expression cassettes and gene delivery vectors described herein, the transfer cassette is a pCCL-SIN-cPPT/CTS-hPGK-hFANCA-WPRE (FIG. 41) of the disclosure comprises or consists of the following sequence, or a sequence having at least 80%, at least 85%, at least 90%, at least 95%, at least 98%, or at least 99% identity to SEQ ID NO: 24. SEQ ID NO: 24 corresponds to the pCCL-PGK-FANCA-WPRE* plasmid of FIG. 41.

In one embodiment, a FANCA gene is delivered via a lentiviral vector (LV). The FANCA LVs described herein utilize a self-inactivating lentiviral vector (LV). In one embodiment, the FANCA LV comprises a promoter of the human phosphoglycerate (PGK) gene. The safety properties of this vector have been markedly improved, compared to the gamma-retroviral vectors already used in the clinics, which harbored strong viral promoters.

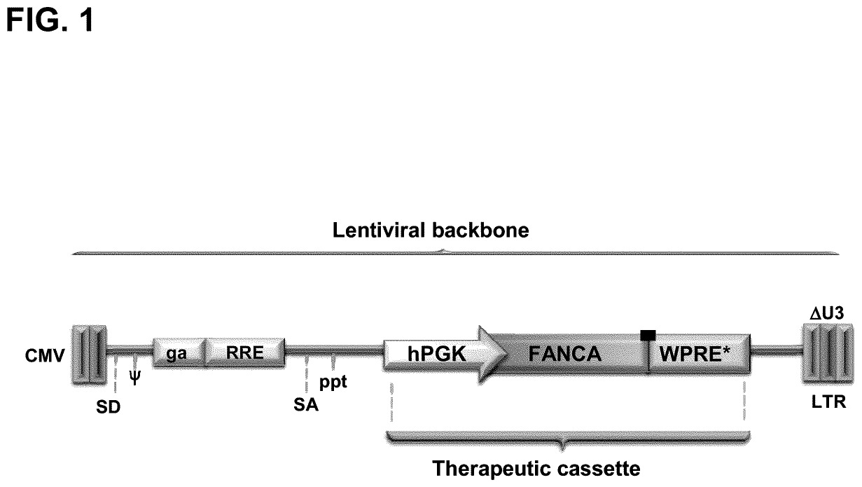

In certain embodiments, the lentiviral vector is PGK-FANCA.WPRE*LV, which comprises the gene transfer cassette depicted in FIG. 1, comprising sequences disclosed in SEQ ID NO: 24. The PGK-FANCA-WPRE*LV gene expression cassette portion comprises the human PGK promoter, the coding sequence for FANCA cDNA, and the WPRE*; and corresponds to nucleotides 3541 to 9178 of SEQ ID NO: 24. The PGK-FANCA-WPRE*LV transfer cassette portion comprises from about the 5′ LTR (U5) to about the 3′ LTR (U5) of the sequence shown in FIG. 41. With respect to SEQ ID NO: 24, nucleotides 1586-1789 of SEQ ID NO: 24 comprise human CMV immediate early promoter. Nucleotides 2031-2156 of SEQ ID NO: 24 comprise HIV1 psi packaging signal. Nucleotides 2649-2882 of SEQ ID NO: 24 comprise HIV1 RRE element. Nucleotides 3378-3495 of SEQ ID NO: 24 comprise HIV cPPT/CTS element. Nucleotides 3541-4051 of SEQ ID NO: 24 comprise the hPGK promoter. Nucleotides 4078-8445 of SEQ ID NO: 24 comprise human FANCA-A cDNA. Nucleotides 8502-9178 of SEQ ID NO: 24 comprise mutated WPRE element. Nucleotides 9262-9495 of SEQ ID NO: 24 comprise the HIV delta U 3′ LTR.

In yet another embodiment, the lentiviral vector contains the following elements:
(i) the backbone of the lentiviral vector derived from the initial pCCLsin-cppt-hPGK-eGFP-WPRE (Dull et al, 1998; J. Virol 72 (11), 9873-9880). The pCCL backbone utilizes a heterologous CMV-HIV 5′ LTR to obtain high levels of viral RNA transcription in the producer cells. Such heterologous LTR renders the construct independent from the need to use the HIV Tat protein for the production of the rHIV particles and it is therefore a safety feature. The U3 region of the 3′ LTR contains a 400 bp deletion as described in (Zufferey et al J Virol, 1998) which confers self inactivating properties to the vector;
(ii) the cDNA of the human FANCA gene (4368 bp GenBank accession number: X_99226 or as disclosed herein) encoding the FANCA protein (1455 AA) under control of the human PGK promoter. The promoter has already been characterized by its stable activity in vivo and by improved safety properties, compared to other promoters already used in gene therapy; and
(iii) a mutated version of the woodchuck hepatitis virus post-transcriptional regulatory element (WPRE) that is deleted in the 3′ region of a sequence coding for the X protein and any residual ORF of the described by Schambach et al (Gene therapy, 2006; 13, 641-645) or WPRE*.

Gene therapy vectors encapsulating the polynucleotide cassettes of the present disclosure may be produced using standard methodology. For example, in the case of LV virions, an LV expression vector according to the invention may be introduced into a producer cell, followed by introduction of an LV helper construct, where the helper construct includes LV coding regions capable of being expressed in the producer cell and which complement LV helper functions absent in the LV vector. This is followed by introduction of helper virus and/or additional vectors into the producer cell, wherein the helper virus and/or additional vectors provide accessory functions capable of supporting efficient LV virus production. The producer cells are then cultured to produce LV. These steps are carried out using standard methodology. In particular embodiments, the plasmids depicted in FIGS. 38-41 are used to produce the gene delivery vectors.

Any suitable method for producing viral particles for delivery of the subject polynucleotide cassettes can be used, including but not limited to those described in the examples that follow. Any concentration of viral particles suitable to effectively transducer mammalian cells can be prepared for contacting mammalian cells in vitro or in vivo. For example, the viral particles may be formulated at a concentration of 10⁸ vector genomes per ml or more, for example, 5×10⁸ vector genomes per mL; 10⁹ vector genomes per mL; 5×10⁹ vector genomes per mL, 10¹⁰ vector genomes per mL, 5×10¹⁰ vector genomes per mL; 10¹¹ vector genomes per mL; 5×10¹¹ vector genomes per mL; 10¹² vector genomes per mL; 5×10¹² vector genomes per mL; 10¹³ vector genomes per mL; 1.5×10¹³ vector genomes per mL; 3×10¹³ vector genomes per mL; 5×10¹³ vector genomes per mL; 7.5×10¹³ vector genomes per mL; 9×10¹³ vector genomes per mL; 1×10¹⁴ vector genomes per mL, 5×10¹⁴ vector genomes per mL or more, but typically not more than 1×10¹⁵ vector genomes per mL.

In preparing the subject LV compositions, any host cells for producing LV virions may be employed, including, for example, mammalian cells (e.g. 293 cells), insect cells (e.g. SF9 cells), microorganisms and yeast. Host cells can also be packaging cells in which the LV rep and cap genes are stably maintained in the host cell or producer cells in which the LV vector genome is stably maintained and packaged. Exemplary packaging and producer cells are derived from SF-9, 293, A549 or HeLa cells. LV vectors are purified and formulated using standard techniques known in the art.

In certain embodiments, the present invention includes a cell comprising a gene expression cassette, gene transfer cassette, or gene delivery vector disclosed herein. In related embodiments, the cell is transduced with a gene delivery vector comprising an expression cassette disclosed herein or has an expression cassette disclosed herein integrated into the cell's genome.

In certain embodiments, the cell is a cell used to produce a viral gene delivery vector, e.g., a packaging cell.

In other embodiments, the cell is a cell to be delivered to a subject in order to provide to the subject the gene product encoded by the expression cassette. Thus, in certain embodiments, the cell is autologous to the subject to be treated or was obtained from the subject to be treated. In other embodiments, the cell is allogeneic to the subject to be treated or was obtained from a donor other than the subject to be treated. In particular embodiments, the cell is a mammalian cell, e.g., a human cell. In certain embodiments, the cell is a blood cell, an erythrocyte, a hematopoietic progenitor cell, a bone marrow cell, e.g., a lineage depleted bone marrow cell, a hematopoietic stem cell (e.g., CD34+) or a committed hematopoietic erythroid progenitor cell. In particular embodiments, the cell is a CD34+ cell obtained from a subject to be treated with the cell after it is transduced by a gene delivery vector disclosed herein. In particular embodiment, the cell is a CD34+ FA cell obtained from a subject diagnosed with FA.

The present invention includes pharmaceutical compositions comprising a polynucleotide cassette, gene delivery vector, or cell described herein and a pharmaceutically-acceptable carrier, diluent or excipient. The subject polynucleotide cassette, gene delivery vector, or cell can be combined with pharmaceutically-acceptable carriers, diluents and reagents useful in preparing a formulation that is generally safe, nontoxic, and desirable, and includes excipients that are acceptable for primate use. Such excipients can be solid, liquid, semisolid, or, in the case of an aerosol composition, gaseous. Examples of such excipients, carriers or diluents include, but are not limited to, water, saline, Ringer's solutions, dextrose solution, and 5% human serum albumin. Supplementary active compounds can also be incorporated into the formulations. Solutions or suspensions used for the formulations can include a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates; detergents such as Tween 20 to prevent aggregation; and compounds for the adjustment of tonicity such as sodium chloride or dextrose. The pH can be adjusted with acids or bases, such as hydrochloric acid or sodium hydroxide. In particular embodiments, the pharmaceutical compositions are sterile.

Pharmaceutical compositions suitable for use in the present invention further include sterile aqueous solutions or dispersions and sterile powders for the extemporaneous preparation of sterile injectable solutions or dispersion.

Sterile solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In one embodiment, the compositions are prepared with carriers that will protect the gene cassette or expression vector against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially.

It is especially advantageous to formulate oral, ocular or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser, e.g. syringe, e.g. a prefilled syringe, together with instructions for administration.

The pharmaceutical compositions of the invention encompass any pharmaceutically acceptable salts, esters, or salts of such esters, or any other compound which, upon administration to an animal comprising a human, is capable of providing (directly or indirectly) the biologically active metabolite or residue thereof.

The term “pharmaceutically acceptable salt” refers to physiologically and pharmaceutically acceptable salts of the compounds of the invention: i.e., salts that retain the desired biological activity of the parent compound and do not impart undesired toxicological effects thereto. A variety of pharmaceutically acceptable salts are known in the art and described, e.g., in in “Remington's Pharmaceutical Sciences”, 17th edition, Alfonso R. Gennaro (Ed.), Mark Publishing Company, Easton, PA, USA, 1985 (and more recent editions thereof), in the “Encyclopaedia of Pharmaceutical Technology”, 3rd edition, James Swarbrick (Ed.), Informa Healthcare USA (Inc.), NY, USA, 2007, and in J. Pharm. Sci. 66: 2 (1977). Also, for a review on suitable salts, see Handbook of Pharmaceutical Salts: Properties, Selection, and Use by Stahl and Wermuth (Wiley-VCH, 2002).

Pharmaceutically acceptable base addition salts are formed with metals or amines, such as alkali and alkaline earth metals or organic amines. Metals used as cations comprise sodium, potassium, magnesium, calcium, and the like. Amines comprise N—N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. Pharma Sci., 1977, 66, 119). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention.

The subject polynucleotide cassette, gene delivery vector, e.g., recombinant virus (virions), or cell (e.g., transduced with a gene delivery vector disclosed herein) can be incorporated into pharmaceutical compositions for administration to mammalian patients, particularly primates and more particularly humans. The subject polynucleotide cassette, gene delivery vector, e.g. virions, or cell can be formulated in nontoxic, inert, pharmaceutically acceptable aqueous carriers, preferably at a pH ranging from 3 to 8, more preferably ranging from 6 to 8. Such sterile compositions will comprise the vector or virion containing the nucleic acid encoding the therapeutic molecule dissolved in an aqueous buffer having an acceptable pH upon reconstitution.

In some embodiments, the pharmaceutical composition provided herein comprise a therapeutically effective amount of a cell, vector or virion disclosed herein in admixture with a pharmaceutically acceptable carrier and/or excipient, for example saline, phosphate buffered saline, phosphate and amino acids, polymers, polyols, sugar, buffers, preservatives and other proteins. Exemplary amino acids, polymers and sugars and the like are octylphenoxy polyethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringer's and Hank's solutions, cysteine, arginine, carnitine, alanine, glycine, lysine, valine, leucine, polyvinylpyrrolidone, polyethylene and glycol. Preferably, this formulation is stable for at least six months at 4° C.

In some embodiments, the pharmaceutical composition provided herein comprises a buffer, such as phosphate buffered saline (PBS) or sodium phosphate/sodium sulfate, tris buffer, glycine buffer, sterile water and other buffers known to the ordinarily skilled artisan such as those described by Good et al. (1966) Biochemistry 5:467. The pH of the buffer in which the pharmaceutical composition comprising the tumor suppressor gene contained in the adenoviral vector delivery system, may be in the range of 6.5 to 7.75, preferably 7 to 7.5, and most preferably 7.2 to 7.4.

In certain embodiments, viral vectors may be formulated into any suitable unit dosage, including, without limitation, 1×10⁸ vector genomes or more, for example, 1×10⁹, 1×10¹⁰, 1×10¹¹, 1×10¹², or 1×10¹³ vector genomes or more, in certain instances, 1×10¹⁴ vector genomes, but usually no more than 4×10¹⁵ vector genomes. In some cases, the unit dosage is at most about 5×10¹⁵ vector genomes, e.g. 1×10¹⁴ vector genomes or less, for example 1×10¹³, 1×10¹², 1×10¹¹, 1×10¹⁰, or 1×10⁹ vector genomes or less, in certain instances 1×10⁸ vector genomes or less, and typically no less than 1×10⁸ vector genomes. In some cases, the unit dosage is 1×10¹⁰ to 1×10¹¹ vector genomes. In some cases, the unit dosage is 1×10¹⁰ to 3×10¹² vector genomes. In some cases, the unit dosage is 1×10⁹ to 3×10¹³ vector genomes. In some cases, the unit dosage is 1×10⁸ to 3×10¹⁴ vector genomes. In one embodiment, the range is from about 5×10¹⁰ to about 1×10¹¹ vector genomes. In some embodiments, the range is from about 1×10⁹ to about 1×10¹⁰ vector genomes.

In some cases, the unit dosage of a pharmaceutical composition may be measured using multiplicity of infection (MOI). By MOI it is meant the ratio, or multiple, of vector or viral genomes to the cells to which the nucleic acid may be delivered. In some cases, the MOI may be 1×10⁶. In some cases, the MOI may be 1×10⁵-1×10⁷. In some cases, the MOI may be 1×10⁴-1×10⁸. In some cases, recombinant viruses of the disclosure are at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI. In some cases, recombinant viruses of this disclosure are 1×10⁸ to 3×10¹⁴ MOI. In some cases, recombinant viruses of the disclosure are at most about 1×10¹, 1×10², 1×10³, 1×10⁴, 1×10⁵, 1×10⁶, 1×10⁷, 1×10⁸, 1×10⁹, 1×0¹⁰, 1×10¹¹, 1×10¹², 1×10¹³, 1×10¹⁴, 1×10¹⁵, 1×10¹⁶, 1×10¹⁷, and 1×10¹⁸ MOI. In some, embodiments the range is from about 20 to about 400 MOI.

In some aspects, the amount of pharmaceutical composition comprises about 1×10⁸ to about 1×10¹⁵ recombinant viruses, about 1×10⁹ to about 1×10¹⁴ recombinant viruses, about 1×10¹⁰ to about 1×10¹³ recombinant viruses, or about 1×10¹¹ to about 3×10¹² recombinant viruses.

### Methods

As discussed in more detail below, the subject polynucleotide cassettes and gene delivery vectors, referred to collectively herein as the “subject compositions”, find use in expressing a transgene, e.g., FANCA, in cells of an animal, e.g., a mammal or human. For example, the subject compositions may be used in research, e.g., to determine the effect that the gene has on cell viability and/or function. As another example, the subject compositions may be used in medicine, e.g., to treat a disorder such as FA. Thus, in some aspects of the invention, methods are provided for the expression of a gene in cells, the method comprising contacting cells with a composition of the present disclosure. In some embodiments, contacting occurs in vitro. In some embodiments, contacting occurs in vivo, i.e., the subject composition is administered to a subject.

For instances in which mammalian cells are to be contacted in vitro or in vivo with a subject polynucleotide cassette or gene delivery vector comprising a subject polynucleotide cassette, the cells may be from any mammalian species, e.g., rodent (e.g., mice, rats, gerbils, squirrels), rabbit, feline, canine, goat, ovine, pig, equine, bovine, primate, human. Cells may be from established cell lines or they may be primary cells, where “primary cells”, “primary cell lines”, and “primary cultures” are used interchangeably herein to refer to cells and cells cultures that have been derived from a subject and allowed to grow in vitro for a limited number of passages, i.e., splittings, of the culture. For example, primary cultures are cultures that may have been passaged 0 times, 1 time, 2 times, 4 times, 5 times, 10 times, or 15 times, but not enough times go through the crisis stage. Typically, the primary cell lines of the present invention are maintained for fewer than 10 passages in vitro.

Embodiments of the present invention comprise mammalian cells (e.g., CD34+ cells) transduced with a viral delivery vector, e.g., a LV vector containing the human FANCA gene. In addition, the present invention includes a method of transducing a mammalian cell, e.g. a human hematopoietic stem cell or other cell described herein, comprising contacting the cell with a gene delivery vector, e.g., a LV vector, disclosed herein or comprising an expression cassette described herein. In certain embodiments, the cell was previously obtained from a subject to be treated, or from another donor. In particular embodiments, the subject was diagnosed with Fanconi Anemia, and the cell is transduced with a LV comprising an expression cassette encoding a FANCA coding region or cDNA. It is understood that the disclosed methods, e.g., those used to deliver a FANCA gene product, e.g., using a FANCA cDNA sequence, to a subject may also be used to treat Fanconi Anemia. In particular embodiments, the transduced cells are a population of cells obtained from a subject with FA, who is to be treated with the cells once they have been transduced. The cells may be obtained from bone marrow or blood. In certain embodiments the subject with FA is treated with agents to mobilize stem cells, then blood is drawn from the subject, red blood cells are removed, and CD34+ cells are selected. Following selection, the cells are then transduced. In particular embodiments, the transduced cells are stored or frozen before use, whereas in certain embodiments, they are provided to the subject immediately or shortly after they are transduced, e.g., within one hour, two hours, or four hours.

In certain embodiments, when transducing a cell with a gene delivery vector disclosed herein, the cells are contacted with the gene delivery vector for about 30 minutes, about 1 hour, about 1.5 hours, about 2 hours, about 2.5 hours, about 3 hours, about 3.5 hours, about 4 hours, about 5 hours, about 6 hours, about 7 hours, about 8 hours, about 12 hours, about 16 hours, about 18 hours, about 20 hours, about 24 hours, about 36 hours, about 48 hours, about 60 hours. In some embodiments, the cells are transduced for less than 60 hours, less than 48 hours, less than 36 hours, or less than 24 hours.

The subject polynucleotide cassette or gene delivery vector comprising a subject polynucleotide cassette may be provided to the subject cells one or more times, e.g. one time, twice, three times, or more than three times, and the cells allowed to incubate with the agent(s) for some amount of time following each contacting event e.g. 16-24 hours, after which time the media is replaced with fresh media and the cells are cultured further. Contacting the cells may occur in any culture media and under any culture conditions that promote the survival of the cells. The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

Typically, an effective amount of subject gene delivery vector or transduced cells comprising a subject polynucleotide cassette is provided to produce the expression of the transgene in cells. As discussed elsewhere herein, the effective amount may be readily determined empirically, e.g. by detecting the presence or levels of transgene gene product, by detecting an effect on the viability or function of the cells, etc. Typically, an effect amount of subject polynucleotide cassette or gene delivery vector comprising a subject polynucleotide cassette will promote greater expression of the transgene in cells than the same amount of a polynucleotide cassette as known in the art. Typically, expression will be enhanced 2-fold or more relative to the expression from a reference, or control, polynucleotide cassette e.g. as known in the art, for example 3-fold, 4-fold, or 5-fold or more, in some instances 10-fold, 20-fold or 50-fold or more, e.g. 100-fold.

For instances in which cells are to be contacted in vivo with a subject polynucleotide cassette or gene delivery vector comprising a subject polynucleotide cassette, the subject may be any mammal, e.g. rodent (e.g. mice, rats, gerbils), rabbit, feline, canine, goat, ovine, pig, equine, bovine, or primate. In a further preferred embodiment, the primate is a human. In a further embodiment, the cells are CD34+ cells.

The methods and compositions of the present disclosure find use, e.g., in the treatment of Fanconi Anemia.

In some embodiments, the subject method results in a therapeutic benefit, e.g., preventing the development of a disorder, halting the progression of a disorder, reversing the progression of a disorder, etc. For example, in one embodiment, the disorder is BMF. In one embodiment, the disorder is thrombocytopenia. In another embodiment, the disorder is leukopenia. In one embodiment, the disorder is pancytopenia. In one embodiment, the disorder is neutropenia. In another embodiment, the disorder is anemia. In some embodiments, the subject method comprises the step of detecting that a therapeutic benefit has been achieved. The ordinarily skilled artisan will appreciate that such measures of therapeutic efficacy will be applicable to the particular disease being modified, and will recognize the appropriate detection methods to use to measure therapeutic efficacy.

In another embodiment, the present invention includes a method of treating a disease in a subject in need thereof comprising providing to the subject an effective amount of cells transduced with a gene delivery vector, e.g., a viral vector, that expresses a therapeutic gene product in the cells. In particular embodiments, the cells are autologous to the subject. In certain embodiments, the cells are erythroid cells, e.g., hematopoietic stem cells or committed hematopoietic erythroid progenitor cells. In some embodiments, the cell is a bone marrow cell, e.g., a lineage depleted bone marrow cell. In particular embodiments, the method is used to treat FA, and the viral vector is a LV comprising an expression construct disclosed herein comprising a human PGK promoter operably linked to a FANCA gene cDNA or coding sequence, and a mutated wPRE disclosed herein. In particular embodiments, the cells are provided to the subject parenterally, e.g., via intravenous injection.

In another embodiment, the present invention includes a method of treating FA in a subject in need thereof, comprising providing to the subject an effective amount of autologous CD34+ stem cells transduced with a LV vector that expresses a FANCA cDNA in the cells, wherein the LV vector comprises a human PGK promoter operably linked to the FANCA cDNA or coding sequence, and a mutated wPRE sequence disclosed herein. In particular embodiments, the cells are hematopoietic stem cells or committed hematopoietic erythroid progenitor cells, e.g., bone marrow cells. In particular embodiments, the cells are provided to the subject parenterally, e.g., via intravenous injection.

Expression of the transgene using the subject transgene is expected to be robust. Accordingly, in some instances, the expression of the transgene, e.g. as detected by measuring levels of gene product, by measuring therapeutic efficacy, etc. may be observed two months or less after administration, e.g. 4, 3 or 2 weeks or less after administration, for example, 1 week after administration of the subject composition. Expression of the transgene is also expected to persist over time. Accordingly, in some instances, the expression of the transgene, e.g. as detected by measuring levels of gene product, by measuring therapeutic efficacy, etc., may be observed 2 months or more after administration of the subject composition, e.g., 4, 6, 8, or 10 months or more, in some instances 1 year or more, for example 2, 3, 4, or 5 years, in certain instances, more than 5 years.

In certain embodiments, the method comprises the step of detecting expression of the transgene in the cells or in the subject, wherein expression is enhanced relative to expression from a polynucleotide cassette not comprising the one or more improved elements of the present disclosure. Typically, expression will be enhanced 2-fold or more relative to the expression from a reference, i.e. a control polynucleotide cassette, e.g. as known in the art, for example 3-fold, 4-fold, or 5-fold or more, in some instances 10-fold, 20-fold or 50-fold or more, e.g. 100-fold, as evidenced by, e.g. earlier detection, higher levels of gene product, a stronger functional impact on the cells, etc.

Typically, if the subject composition is an LV comprising the subject a polynucleotide cassette of the present disclosure, an effective amount to achieve a change in will be about 1×10⁸ vector genomes or more, in some cases 1×10⁹, 1×10¹⁰, 1×10¹¹, 1×10¹², or 1×10¹³ vector genomes or more, in certain instances, 1×10¹⁴ vector genomes or more, and usually no more than 1×10¹⁵ vector genomes. In some cases, the amount of vector genomes that is delivered is at most about 1×10¹⁵ vector genomes, e.g. 1×10¹⁴ vector genomes or less, for example 1×10¹³, 1×10¹², 1×10¹¹, 1×10¹⁰, or 1×10⁹ vector genomes or less, in certain instances 1×10⁸ vector genomes, and typically no less than 1×10⁸ vector genomes. In some cases, the amount of vector genomes that is delivered is 1×10¹⁰ to 1×10¹¹ vector genomes. In some cases, the amount of vector genomes that is delivered is 1×10¹⁰ to 3×10¹² vector genomes. In some cases, the amount of vector genomes that is delivered is 1×10⁹ to 3×10¹³ vector genomes. In some cases, the amount of vector genomes that is delivered is 1×10⁸ to 3×10¹⁴ vector genomes.

In some cases, the amount of pharmaceutical composition to be administered may be measured using multiplicity of infection (MOI). In some cases, MOI may refer to the ratio, or multiple of vector or viral genomes to the cells to which the nucleic may be delivered. In some cases, the MOI may be 1×10⁶. In some cases, the MOI may be 1×10⁵-1×10⁷. In some cases, the MOI may be 1×10⁴-1×10⁸. In some cases, recombinant viruses of the disclosure are at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI. In some cases, recombinant viruses of this disclosure are 1×10⁸ to 3×10¹⁴ MOI. In some cases, recombinant viruses of the disclosure are at most about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI.

In some aspects, the amount of pharmaceutical composition comprises about 1×10⁸ to about 1×10¹⁵ particles of recombinant viruses, about 1×10⁹ to about 1×10¹⁴ particles of recombinant viruses, about 1×10¹⁰ to about 1×10¹³ particles of recombinant viruses, or about 1×10¹¹ to about 3×10¹² particles of recombinant viruses.

Any total number of viral particles suitable to provide appropriate transduction of cells to confer the desired effect or treat the disease can be administered to the mammal. In various preferred embodiments, at least 10⁸; 5×10⁸; 10⁹; 5×10⁹, 10¹⁰, 5×10¹⁰; 10¹¹; 5×10¹¹; 10¹²; 5×10¹²; 10¹³; 1.5×10¹³; 3×10¹³; 5×10¹³; 7.5×10¹³; 9×10¹³, 1×10¹⁴ viral particles, or 5×10¹⁴ viral particles or more, but typically not more than 1×10¹⁵ viral particles are injected. Any suitable number of administrations of the vector to the mammal or the primate eye can be made. In one embodiment, the methods comprise a single administration; in other embodiments, multiple administrations are made over time as deemed appropriate by an attending clinician. In some embodiments at least 2×10⁸ VG/ml of 5×10⁵ cells/ml is required in a single administration (24 hours transduction) to result in high transduction efficiencies. Individual doses are typically not less than an amount required to produce a measurable effect on the subject, and may be determined based on the pharmacokinetics and pharmacology for absorption, distribution, metabolism, and excretion (“ADME”) of the subject composition or its by-products, and thus based on the disposition of the composition within the subject. This includes consideration of the route of administration as well as dosage amount. Effective amounts of dose and/or dose regimen can readily be determined empirically from preclinical assays, from safety and escalation and dose range trials, individual clinician-patient relationships, as well as in vitro and in vivo assays such as those described herein and illustrated in the Examples.

In some embodiments, the dose of cells patients receive by infusion will be that which is obtained from the transduction process. In various preferred embodiments, at least at least about 1×10¹, 1×10², 1×10³, 1×10⁴, 1×10⁵, 1×10⁶, 1×10⁷, 1×10⁸, or more CD34+ cells/KG of patient weight are infused into the patient. In some embodiments, between 1×10⁶ and 4×10⁶ CD34+ cells/KG of patient weight are infused into the patient. In other embodiments, 3×10⁵ and 4×10⁶ CD34⁺ cells/Kg of patient weight are infused into the patient. In some embodiments, cells will be infused into the patient a single dose. In other embodiments, cells will be infused into the patient in multiple doses. Transduced cells may be infused immediately after the transduction process is completed.

Once integrated, the therapeutic protein (e.g., human FANCA protein) is expressed by the cells. Transduced FA cells are genetically corrected, and thus able to activate the FA pathway by the mono-ubiquitination of FANCD2 and FANCI. These proteins migrate to areas of DNA damage, and in cooperation with other DNA repair proteins, promote the repair of the DNA in these cells, as occurs in healthy cells

As described in further detail in the Examples, preclinical in vitro data with BM samples from human FA patients has already shown the efficacy of an FANCA LV to correct the phenotype of these cells.

Accordingly, the present invention provides methods for treatment of the hematological manifestations of FA. In one embodiment, the hematological manifestation of FA is selected from one or more of BMF, thrombocytopenia, leukopenia, pancytopenia, neutropenia, and anemia. In a particular embodiment, the hematological manifestation is bone marrow failure (BMF), which appears in pediatric ages in most FA patients. In one embodiment, the hematological manifestation is thrombocytopenia. In another embodiment, the hematological manifestation is leukopenia. In one embodiment, the hematological manifestation is pancytopenia. In one embodiment, the hematological manifestation is neutropenia. In another embodiment, the hematological manifestation is anemia. In one embodiment, the hematological manifestation is a combination of two or more of BMF, thrombocytopenia, leukopenia, pancytopenia, neutropenia, and anemia.

An FANCA LV does not directly treat solid tumors that may be generated in more advanced stages of the disease. Nevertheless, the improvement of the hematological status of FA patients treated by hematopoietic gene therapy may also improve the immunological surveillance against the development of solid tumors. Therefore, an indirect antitumor effect may also be generated as a consequence of the treatment of FA patients with an FANCA LV.

In order to achieve successful gene therapy in FA, it is beneficial to collect from a subject a “sufficient” number of hematopoietic stem cells (HSC).

In one embodiment, HSCs are obtained, or collected, from a bone marrow sample. In one embodiment, the bone marrow sample is depleted of erythrocytes. In some embodiments, the bone marrow sample is depleted of CD16+ white blood cells. In some embodiments, the cells remaining after depletion techniques are washed. In another embodiment, non-specific IgG is added to the washed cells. In some embodiments, the non-specific IgG is flebogamma. Subsequently, CD34+ cells may be selected from the washed cells. In one embodiment, CD34+ cells are selected from the bone marrow sample. Selection methods for CD34+ cells may be positive selection, negative selection, or a combination thereof.

In another embodiment, HSCs are obtained from peripheral blood. In one embodiment, the peripheral blood sample is depleted of erythrocytes. In some embodiments, the blood sample is depleted of CD16+ white blood cells. In some embodiments, the blood cells remaining after depletion techniques are washed. In another embodiment, non-specific IgG is added to the washed cells. In some embodiments, the non-specific IgG is flebogamma. Subsequently, CD34+ cells may be selected from the washed cells. In one embodiment, CD34+ cells are selected from the peripheral blood sample. Selection methods for CD34+ cells may be positive selection, negative selection, or a combination thereof.

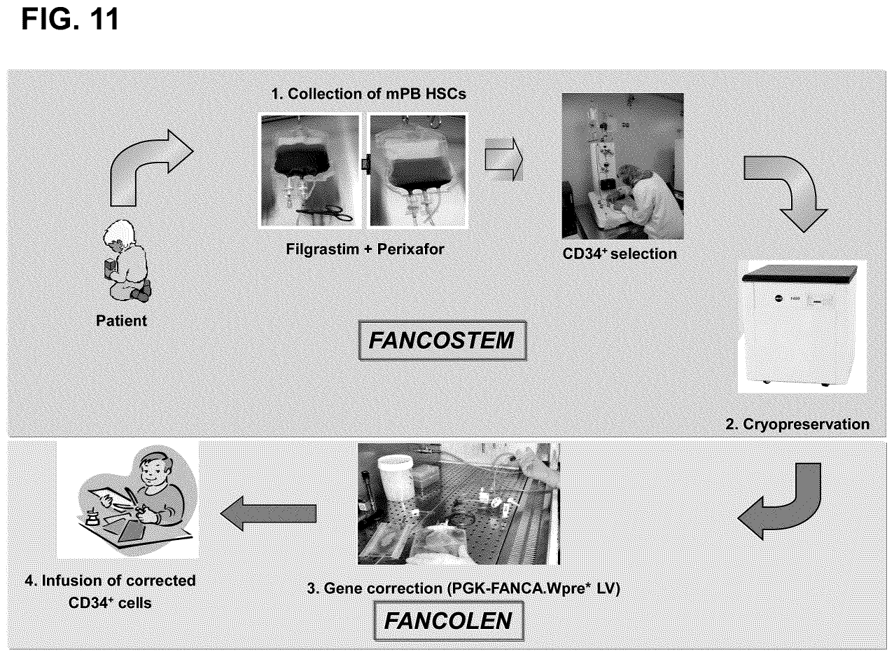

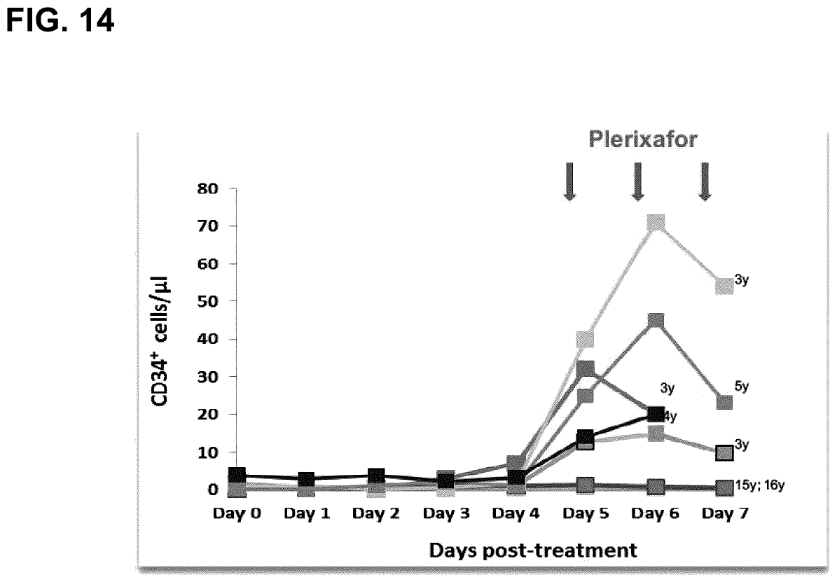

In some embodiments of the present invention, the HSCs are obtained from a subject following mobilization. Mobilization may be achieved by treating the subject with drugs or compounds that cause the movement of stem cells from the bone marrow into the blood. The stem cells can be collected and stores. In some embodiments, mobilization is achieved by treating the subject with G-CSF (filgrastim). In other embodiments, mobilization is achieved by treating the subject with plerixafor. In yet other embodiments, mobilization is achieved by treating the subject with a combination of filgrastim and plerixafor. (FIG. 11 and FIG. 14)

In one embodiment, at least 1 to 4×10⁶ CD34⁺ corrected cells (e.g., FANCA transduced HSCs) per kilogram of patient weight are administered to restore haematopoiesis in a non-conditioned FA patient. In some embodiments, the transduced cells are infused or administered into the patient immediately after transduction. (FIG. 11) In other embodiments, the transduced cells are frozen prior to infusing or administering into the patient. (FIG. 11)

The genetic correction of HSCs from FA patients, followed by the autologous transplantation of these cells (hematopoietic gene therapy), is a good alternative for FA patients, particularly those lacking an HLA-identical sibling. In one embodiment, hematopoietic gene therapy is the preferred treatment regimen for a patient lacking an HLA-identical sibling. In another embodiment, hematopoietic gene therapy is the preferred treatment regimen for a patient that has an HLA-identical sibling.

All publications mentioned herein are incorporated herein by reference to disclose and describe the methods and/or materials in connection with which the publications are cited. It is understood that the present disclosure supersedes any disclosure of an incorporated publication to the extent there is a contradiction.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed.

Because FA-A is the most frequent complementation group in FA patients (Casado et al., 2007, Taniguchi et al., 2006), vectors expressing the FANCA gene and/or the EGFP marker gene are the focus of the Examples; however, other FANCA genes may be utilized to similarly treat other complementation groups.

### EXAMPLES

### Example 1

### FANCA Lentiviral Vectors

Because of the safer integration pattern of LVs compared to RVs (Gonzalez-Murillo et al., 2008; Modlich et al., 2009; Montini et al., 2006; Mitchell R S, Beitzel B F, Schroder A R et al. Plos Biol. 2004; 2:E234; Montini E et al. J Clin Invest. 2009; 119:964-975; Schroder A R et al. Cell. 2002; 110:521-529), it was an aim to develop LVs as therapeutic vectors to correct the phenotype of FA cells (Gonzalez-Murillo et al., 2009). Additionally, since recent studies have shown that LVs harboring potent internal promoters can also trans-activate neighboring genes (Modlich et al., 2009), LVs were considered for use in the clinic as a compromise between therapeutic efficacy and risks to trans-activate neighboring genes. The objective was, therefore, to define threshold levels of FANCA expression that could be therapeutic, in order to limit risks of gene trans-activation by the enhancer/promoter driving the expression of the therapeutic gene. The efficacy of the FANCA LV was first verified in vitro in FA-A LCLs, thereafter in primary BM samples from FA-A patients, and finally in vivo in a mouse model of FA-A.

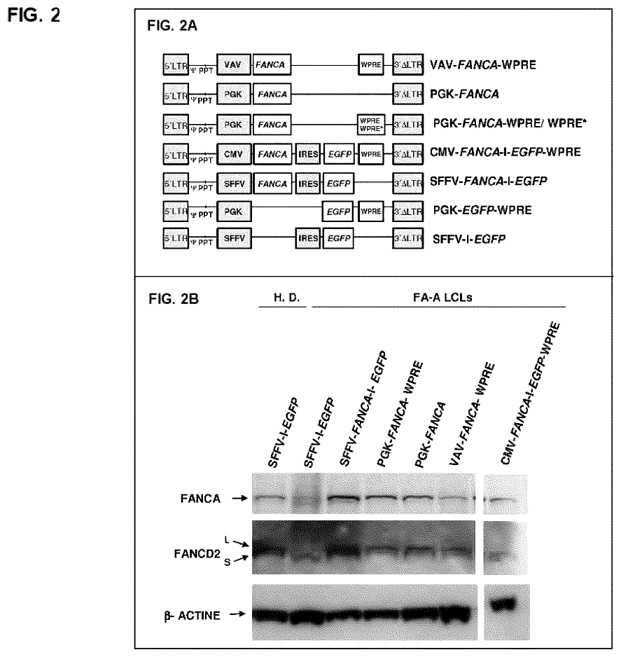

To this aim, LVs expressing FANCA under the control of different promoters: vav, PGK, CMV and SFFV promoters, were constructed. FIG. 1 is a schematic of the medicinal product. FIG. 2A shows a schematic representation of LVs expressing FANCA under the control of different internal promoters. Additionally we also investigated the influence of post-transcriptional WPRE elements, both in the expression level of FANCA and the therapeutic efficacy of the LVs. Initially, all LVs were packaged with the chimeric GALV-TR envelope. Titers of 1-2×10⁶ tu/ml were routinely obtained, and transductions conducted at estimated MOIs of 1-2 tu/cell. FIG. 2B shows a Western blot analysis of FANCA in FA-A cells transduced.

In order to determine the level of FANCA mRNA that was conferred by each vector, transduced FA-A lymphoblast cell lines (LCL) were selected with 30 nM MMC for 5 days. After the selection process, transduced FA-A LCLs contained 0.81 to 3.04 copies of the respective LV per cell (Table 1). Unselected FA-A LCLs transduced with EGFP-LVs and LCLs from a healthy donor (HD) were used as controls.

Total FANCA mRNA levels, as well as relative FANCA mRNA levels per LV copy number were determined in LCLs transduced with the different LVs (Table 1). Compared to FANCA mRNA levels observed in HD LCLs, similar levels of FANCA mRNA/copy were observed in FA-A cells transduced with vav-FANCA and PGK-FANCA LVs. CMV-FANCA and more significantly SFFV-FANCA LVs conferred supra-physiological levels of FANCA mRNA/copy (3.6 and 5.6 fold, respectively). PGK-FANCA LVs harboring the WPRE or the mutated WPRE* sequences increased FANCA mRNA levels 2.3-2.6 fold compared to PGK-LVs without WPRE. Consistent with other studies (Schambach et al., 2006; Zanta-Boussif M A, Charrier S, Brice-Ouzet A Et al. Validation of a Mutated Pre Sequence Allowing High and Sustained Transgene Expression While Abrogating WHV-X Protein Synthesis: Application to the Gene Therapy of WAS. Gene Ther. 2009; 16:605-619; Zufferey R, Donello J E, Trono D, Hope T J. Woodchuck Hepatitis Virus Posttranscriptional Regulatory Element Enhances Expression of Transgenes Delivered by Retroviral Vectors. J Virol. 1999; 73:2886-2892), the insertion of the WPRE or WPRE* sequences significantly increased FANCA mRNA levels in cells transduced with PGK-FANCA LVs. Since the wild type WPRE element encodes for a C-terminal truncated version of the hepatitis virus X protein which could mediate a tumorogenic effect (Bouchard M J, Schneider R J. The Enigmatic X Gene Of Hepatitis B Virus. J Virol. 2004; 78:12725-12734.2004; Kingsman S M, Mitrophanous K, Olsen J C. Potential Oncogene Activity of the Woodchuck Hepatitis Post-Transcriptional Regulatory Element (WPRE). Gene Ther. 2005; 12:3-4), the LV with the mutated WPRE* sequence, which lacks any residual open reading frame is considered more adequate for clinical uses.

TABLE 1
FANCA expression levels in FA-A LCLS transduced with FANCA-expressing lentiviral vectors.

| Cell | | FANCA | mRNA | | Protein | |
|---|---|---|---|---|---|---|
| Donor | Lentiviral Vector | copies/cell | Total FANCA | FANCA/Copy | Total FANCA | FANCA/Copy |
| HD | — | 2* | 1 | 0.5 | 1 | 0.5 |
| FA-A | SFFV-I-EGFP | 0 | 0.27 ± 0.03 | — | — | — |
| FA-A | VAV-FANCA-WPRE | 1.1 ± 0.33 | 0.51 ± 0.01 | 0.57 ± 0.20 | 0.75 | 0.59 |
| FA-A | PGK-FANCA | 3.04 ± 1.36 | 1.92 ± 1.32 | 0.54 ± 0.11 | 1.3 ± 0 | 0.53 ± 0.24 |
| FA-A | PGK-FANCA-WPRE | 3.1 ± 0.30 | 3.91 ± 2.19 | 1.24 ± 0.60 | 1.74 ± 0.28 | 0.57 ± 0.03 |
| FA-A | PGK-FANCA-WPRE * | 1.52 ± 0.05 | 2.20 ± 0.70 | 1.43 ± 0.41 | 1.7 | 0.54 |
| FA-A | CMV-FANCA-I-EGFP | 1.4 ± 0.28 | 2.42 ± 0.06 | 1.8 ± 0.41 | 0.96 | 0.58 |
| FA-A | SFFV-FANCA-I-EGFP | 0.81 ± 0.18 | 2.13 ± 0.26 | 2.78 ± 0.36 | 1.6 | 1.7 |

* To estimate levels of FANCA mRNA and FANCA protein per copy of FANCA, 2 copies of genomic FANCA were considered in healthy donor LCLs.

To test whether differences in FANCA mRNA expression were confirmed at the protein level, Western blot analyses (FIG. 2B) were conducted with samples shown in Table 1. As it was done with FANCA mRNA determinations, FANCA protein values were related not only to protein loadings, but also to the provirus copy number determined in each transduced FA-LCL. Compared to FANCA levels/copy determined in HD-LCLs, essentially normal levels of FANCA/copy were conferred by all tested FANCA-LVs, except by the SFFV-FANCA LV. In this case, relative FANCA levels/copy were 3.4 fold higher to levels determined in HD-LCLs. Western blots re-stained with anti-FANCD2 showed that, while FA-A LCLs transduced with the control vector were not able to mon-oubiquitinate FANCD2, FA-A LCLs transduced with either type of FANCA-LVs expressed both the non-ubiquitinated as well as the mono-ubiquitinated forms of FANCD2, consistent with a functional FA pathway in these cells.

When FANCA expression levels conferred by the different LVs in FA-A LCLs were compared with those observed in healthy donor (HD) LCLs (with two copies of FANCA), we concluded that the insertion of two LV copies per cell may result in physiological levels of the therapeutic protein, except in the case of SFFV-LVs, which would confer supra-physiological levels of FANCA. Achieving this copy number may fit the requirements of a clinical trial of FA patients, where transduction efficacies of at least 50% may be desired because of the low number of progenitors present in the BM of these patients (Gonzalez-Murillo et al., 2009, Jacome et al., 2006, Kelly et al., 2007; Larghero J, Marolleau J P, Soulier J et al. Hematopoietic Progenitor Cell Harvest and Functionality in Fanconi Anemia Patients. Blood. 2002; 100:3051).

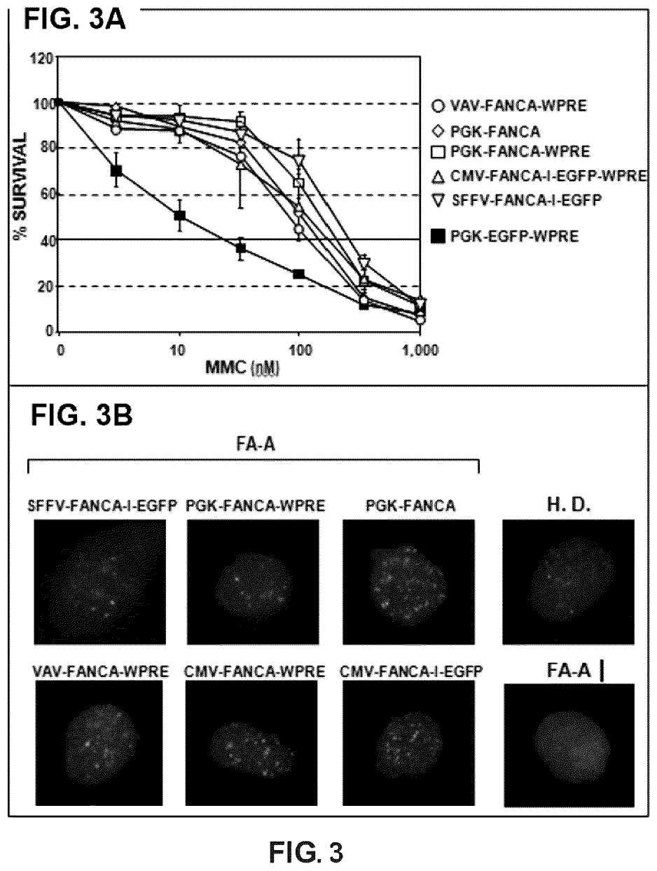

In order to analyze possible differences in the therapeutic efficacy of the different FANCA-expressing LVs, the efficiency of each vector to correct the MMC-hypersensitivity of FA-A lymphoblast cell lines (LCLs) was determined. To this aim, FA-A LCLs were transduced with the different LVs (FIG. 3A) and then exposed to increasing concentrations of MMC. Thereafter, the viability of transduced cells was determined. As shown in FIG. 3A, all tested FANCA-LVs were equally efficient to revert the hypersensitivity of FA-A LCLs. Similarly, all vectors promoted the generation of nuclear FANCD2 foci in MMC-treated cells (FIG. 3B), consistent with a functional FA pathway in FA-A cells transduced with either FANCA-LV.

### Example 2 Mouse Model Studies

To evaluate the repopulating properties of BM samples from FA patients, either genetically corrected or not, several groups have transplanted BM cells from FA patients into immuno-deficient mice. Nevertheless, in no instance have significant engraftments been reported, most probably because of the reduced number of hematopoietic progenitors and HSCs present in the BM of these patients.

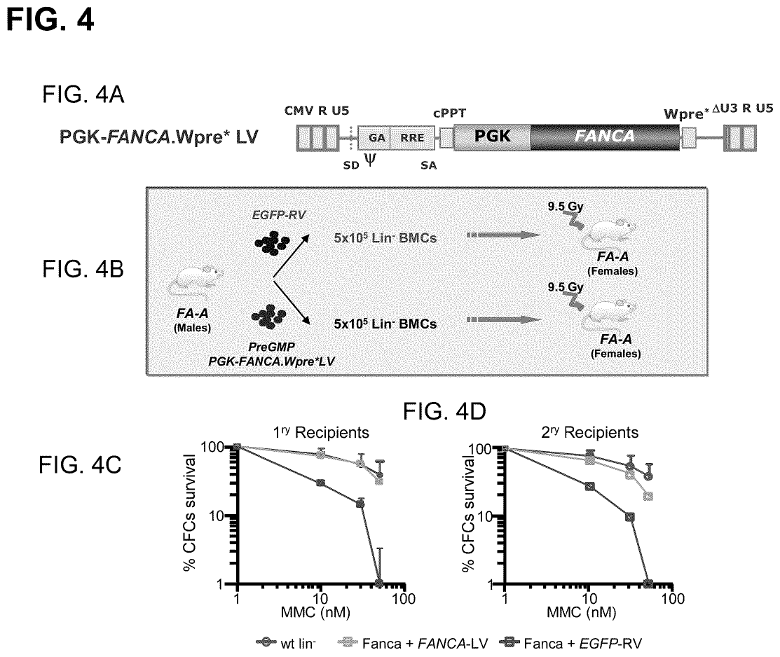

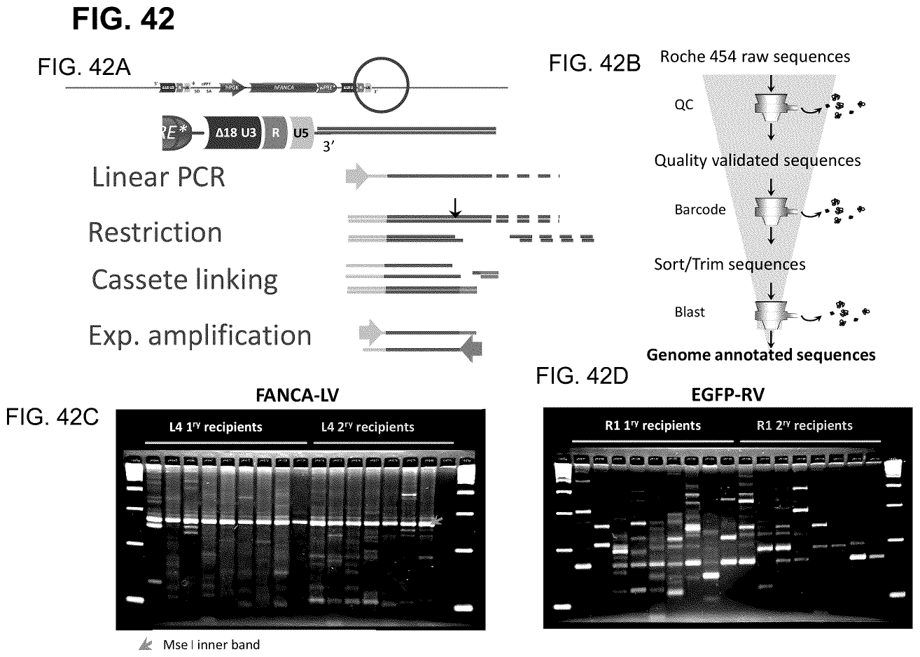

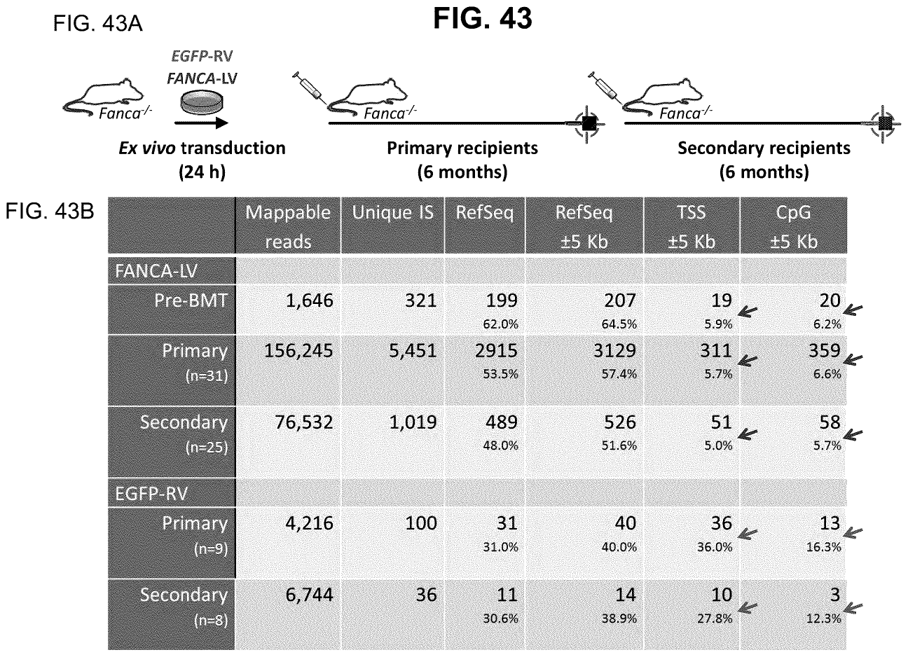

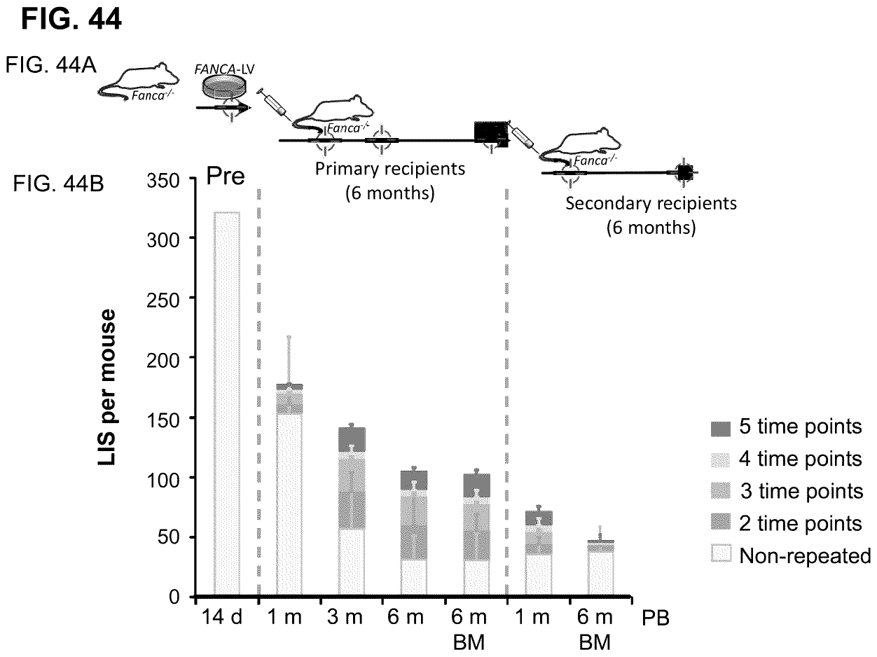

To evaluate the in vivo effects of the medicinal product, a mouse model for FA-A, which contains deletion in the Fanca gene, was used. In contrast to FA patients, these animals do not develop evident hematological defects. Nevertheless, their BM progenitor cells are highly sensitive to MMC (Rio et al., 2002), as it is also the case in FA patients. Therefore, to determine whether the FANCA LV (FIG. 4A) stably corrected the phenotype of the HSCs from FA-A mice in vivo, BM cells from FA-A mice were transduced with FANCA LV and then transplanted into irradiated FA-A recipients (FIG. 4B). To evaluate whether after the transplantation of genetically-corrected cells the phenotype of the hematopoietic progenitors was corrected, BM samples from transplanted FA-A mice were cultured in methylcellulose in the absence and the presence of MMC (FIG. 4C). Additional data confirming the integration of the therapeutic cassette is presented in FIGS. 42-44. Linear Amplification Mediated (LAM) PCR is a method to retrieve the integration sites of different integrating vectors into the genome. A PCR product that starts from the known sequence of the vector and extends through the unknown flanking genome is generated and sequenced to identify the position within the genome of the vector integration. FIG. 42 represents the LAM-PCR analysis of FANCA-LV insertion sites in FA hematopoietic stem cells (HSC). FIG. 43 depicts LAM-PCR results for tracking of FANCA-LV treated cells. FIG. 44 shows the clonal diversity of Fanca−/− recipients transplanted with LV-corrected HSCs.

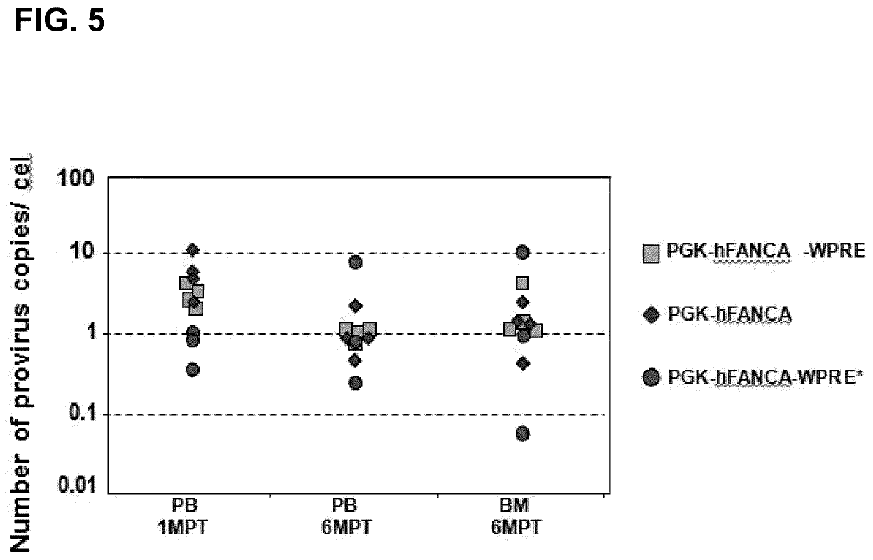

After 1 month post-transplantation normal hematological counts were observed in these animals, showing no evident toxicity of the LV. At this time, the number of proviral FANCA copies per cell varied between 0.5 to 10 copies/cell (similar results were observed at 3 and 6 months post-transplantation) (FIG. 5). To evaluate whether after the transplantation of genetically-corrected cells the phenotype of the hematopoietic progenitors was corrected, BM samples from transplanted FA-A mice were cultured in methylcellulose in the absence and the presence of MMC.

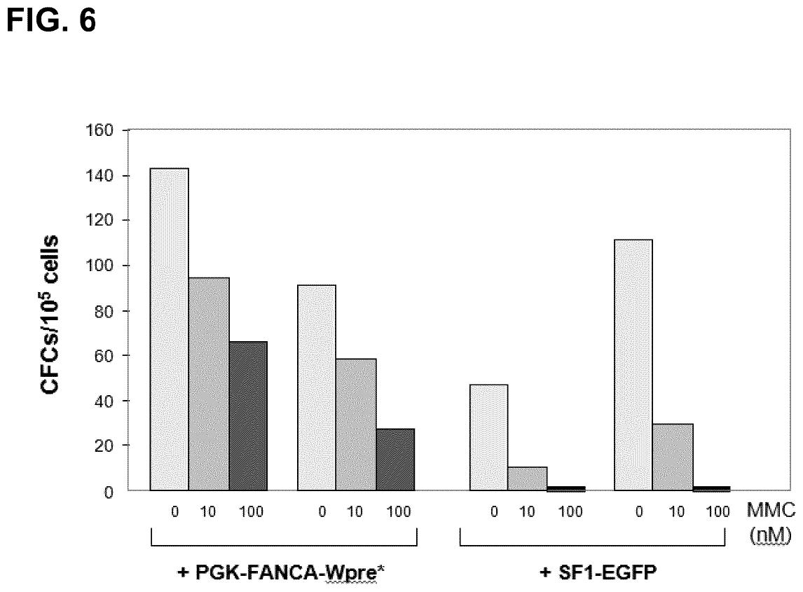

As shown in FIG. 6, a significant correction of the MMC-hypersensitivity was observed in the hematopoietic progenitors from FA mice that were subjected to gene therapy with FANCA LV as compared to the SF1-EGFP LV control. FA-A bone marrow (BM) cells were transduced with PGK_FANCA-WPRE* or control SF1-EGFP LVs and transplanted into irradiated FA-A mice. At 7 months post-transplantation, BM samples were harvested and cultured in methylcellulose in the presence of increasing concentrations of mitomycin C (MMC).

These data therefore indicate that FANCA LV can revert the phenotype of hematopoietic progenitors from FA-A mice after in vivo transplantation. In safety terms, none of the 30 mice that were transplanted with BM cells previously transduced with FANCA LV have developed symptoms of myeloproliferative disorders or leukemia (data obtained up to 1 year post-transplantation).

### Example 3. Efficient Transduction of Fresh Hematopoietic Progenitors from FA Patients with Lentiviral Vectors

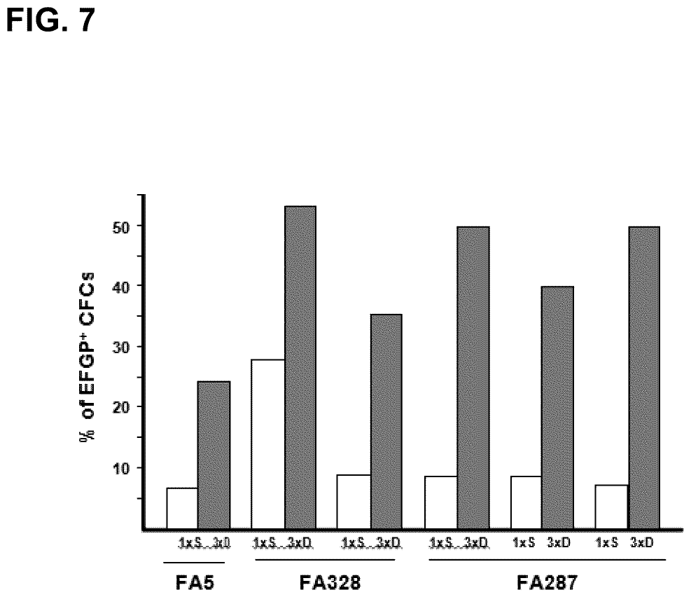

Because transduction of FA cryopreserved hematopoietic stem cells (HSC) was previously shown to be less efficient compared to transduction of fresh grafts (Jacome et al., 2009), an effort to optimize the efficacy of the transduction of cryopreserved FA BM samples was undertaken. As shown in FIG. 7, conducting three transduction cycles significantly increased the transduction efficacy of FA CFCs compared to values obtained after a single transduction cycle (45.7±4.2% versus 13.5±5.10%, respectively). Samples were subjected to standard transductions consisting in a single transduction cycle (16 h) after 2 h of static preloading (white bars; 1×S) or improved transduction consisting in three transduction cycles (2 h+2 h+12 h) with the lentiviral vectors (grey bars; 3×D).

### Example 4. PGK-FANCA-WPRE* Lentiviral Vector Efficiently Corrects the Phenotype of Bone Marrow Progenitors from FA-A Patients

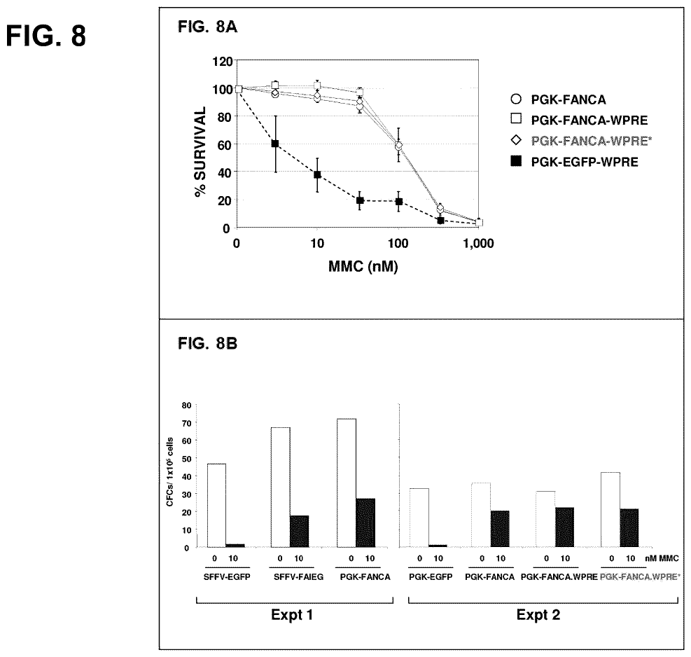

Because of the efficacy of LVs in which FANCA was driven by the PGK promoter, and based on previous studies showing the stability (Follenzi A et al. Nat Genet. 2000; 25:217-222) and low genotoxic properties of LVs carrying the PGK promoter (Modlich et al., 2009, Montini et al., 2006, Montini et al., 2009) further experiments were conducted in LCLs and also in bone marrow cells from FA-A patients using PGK-FANCA LVs, free from any WPRE element or harboring the WPRE or WPRE* sequences. As shown in FIG. 8A, all these three vectors conferred the same reversion in the hypersensitivity of FA-A LCLs to MMC.

To compare the efficacy of SFFV-FANCA and PGK-FANCA LVs to correct the phenotype of hematopoietic progenitors from FA-A patients, erythrocyte-depleted BM samples from FA-A patients were transduced as recently described (Jacome et al., 2009). Erythrocyte-depleted BM cells were transduced for 16 h in plates preloaded with LV supernatants as recently described (Gonzalez-Murillo et al., 2009). Fourteen days later, the number of colonies grown in the absence and the presence of 10 nM MMC was scored to determine the proportion of progenitors that became resistant to the drug.

As shown in FIG. 8B, when samples were transduced with EGFP-LVs almost no colonies were generated in the presence of MMC. In contrast to this observation, the transduction of FA-A BM cells with SFFV-FANCA and PGK-FANCA LVs allowed the growth of 26 and 38% of the colonies scored in cultures without MMC. The efficacy of PGK-FANCA LVs harboring the WPRE and WPRE* sequences was compared to WPRE-free PGK-FANCA LVs. As shown in FIG. 8B, all the three LVs mediated a high and similar level of protection to MMC. Although the insertion of the post-transcriptional regulatory element WPRE* was not necessary for improving the efficacy of PGK-FANCA LVs, this element will offer a redundant element to maintain in the long-term therapeutic levels of ectopic FANCA in the patient.

Taken together, data obtained in these studies strongly suggest that PGK-FANCA-WPRE* LVs confer sufficient levels of FANCA expression to correct the hematopoietic phenotype of FA-A patients. These results, together with previous observations showing the stability (Follenzi et al., 2000) and safety properties of PGK-LVs (Modlich et al., 2009, Montini et al., 2006, Montini et al., 2009) and the efficacy of the mutated WPRE* post-transcriptional element, reinforces the hypothesis that the PGK-FANCA-WPRE* LV may constitute an efficient and safe vector for the gene therapy of FA-A patients.

### Example 5. Analysis of the Efficacy of the GALV-TR and VSV-G Packaged Lentiviral Vectors to Transduce Hematopoietic Progenitors from FA Patients

Because LVs can be pseudotyped both with GALV-TR and VSV-G envelopes, new experiments were conducted in which unselected bone marrow cells from FA patients were transduced under optimized conditions with EGFP-LVs pseudotyped in the two envelopes.

For GALV-TR pseudotyped LVs, two transduction cycles with nonconcentrated LVs (estimated titer: 2×10⁵ IUs/mL; estimated MOI: 2 IUs/cell) were conducted.

For VSV-G pseudotyped LVs, one transduction cycle with concentrated and purified LVs (estimated titer: 10⁸ IUs/mL; estimated MOI: 50 IUs/cell).

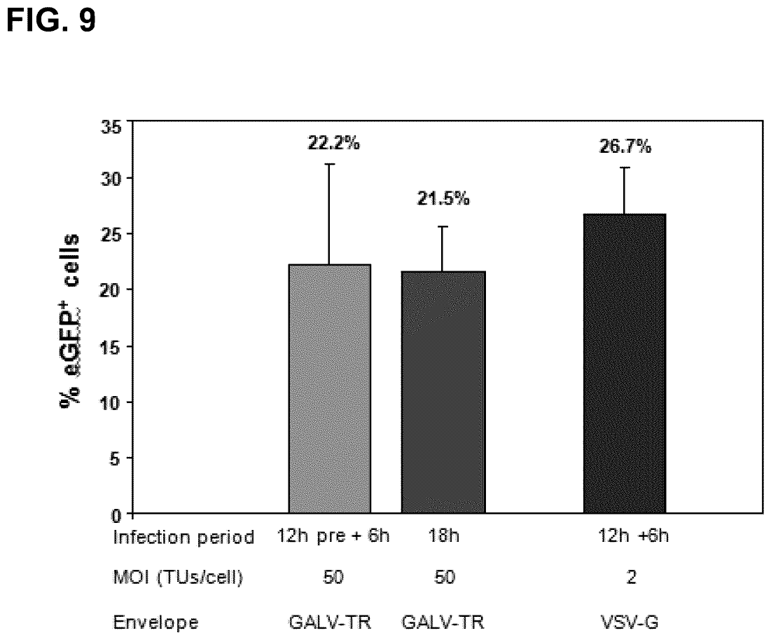

As shown in FIG. 9, under both conditions similar transductions of hematopoietic progenitors from FA patients were obtained, indicating that manufacturing compromises can determine the best envelope for packaging.

### Example 6. Safety Studies

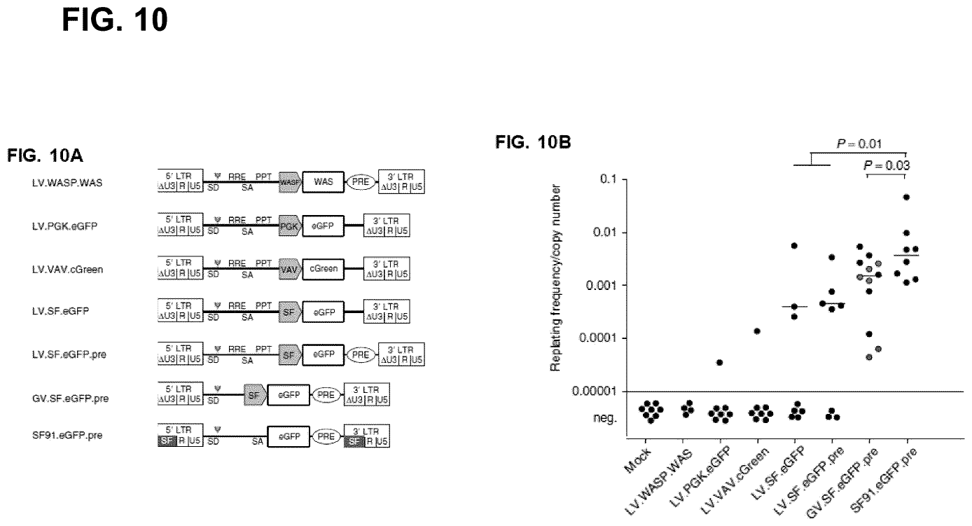

The transforming potential of the lentiviral vectors shown in FIG. 10A was measured in replating frequency over copy number. As shown in FIG. 10B, the transforming potential of the lentiviral backbone corresponding to the PGK-FANCA-WPRE* LV (a PGK-derived lentiviral vector) is markedly lower compared to the transformation capacity of vectors harboring viral promoters, which are the ones already used in the X1-SCID and CGD clinical trials.

With respect to in vivo studies with mice transplanted with BM cells transduced with the PGK-FANCA-WPRE* LV, 30 mice have been transplanted, and so far (up to 1 year post-transplantation) none of the transplanted animals have developed symptoms of myeloproliferative disorders or leukemia.

### Example 7. Pharmaceutical Product

The FANCA lentiviral vector is a third generation self-inactivated rHIV1-derived vector encoding the human FANCA cDNA under control of the human PGK promoter and regulated at the post-transcriptional level by a mutated WPRE lacking the X protein ORF (See FIG. 1, FIG. 41, and SEQ ID NO: 24). Such a FANCA lentiviral vector presents several advantages over the gamma-retroviral vectors previously used in FA gene therapy, notably the ability to transduce cells in spite of short pre-activation protocols which is advantageous to preserve the multi-lineage potential of hematopoietic stem cells.

For the generation of the therapeutic lentiviral vector, 293T cells transfected with three additional plasmids, providing all the required helper proteins for the packaging of the vector (See FIGS. 38-40) will comprise the final packaging of the PGK-FANCA-WPRE* medicinal product (FIG. 41).

The PGK-FANCA-WPRE*LV therapeutic cassette comprises the human PGK promoter, the coding sequence for FANCA cDNA, and the WPRE* enhancer and comprises nucleotides 3541 to 9178 of SEQ ID NO: 24. The region of the transfer cassette comprising the human CMV immediate early promoter, the HIV packaging sequence, the ga and RRE elements, the therapeutic cassette, and the HIV self inactivating 3′LTR wherein the therapeutic cassette comprises the human PGK promoter, the coding sequence for FANCA cDNA, and the WPRE* enhancer is coded for by nucleotides 1586-9495 of SEQ ID NO: 24.

Nucleotides 1586-1789 of SEQ ID NO: 24 comprise human CMV immediate early promoter. Nucleotides 2031-2156 of SEQ ID NO: 24 comprise HIV1 psi packaging signal. Nucleotides 2649-2882 of SEQ ID NO: 24 comprise HIV1 RRE element. Nucleotides 3378-3495 of SEQ ID NO: 24 comprise HIV cPPT/CTS element. Nucleotides 3541-4051 of SEQ ID NO: 24 comprise the hPGK promoter. Nucleotides 4078-8445 of SEQ ID NO: 24 comprise human FANCA-A cDNA. Nucleotides 8502-9178 of SEQ ID NO: 24 comprise mutated WPRE element. Nucleotides 9262-9495 of SEQ ID NO:24 comprise the HIV delta U 3′ LTR.

### Example 8. Clinical Study FANCOSTEM

In the U.S., two gene therapy trials have been conducted in FA-A and FA-C patients, which showed no clinical efficacy (Liu, J. M., et al. (1999). Engraftment of hematopoietic progenitor cells transduced with the Fanconi anaemia group C gene (FANCC). Hum. Gene Ther. 10: 2337-2346; Kelly, P. F., et al. (2007). Stem cell collection and gene transfer in fanconi anaemia. Mol Ther 15: 211-219). The efficacy of the aforementioned trials could be significantly improved through various optimizations. Two clinical trials were conducted in tandem to determine the feasibility of a process for collecting and purifying a sufficient number of CD34⁺ cells for future clinical use (FANCOSTEM) and, in parallel, to evaluate the safety and efficacy of the gene therapy in patients with FA complementation group A (FA-A) (FANCOLEN) See FIG. 11.

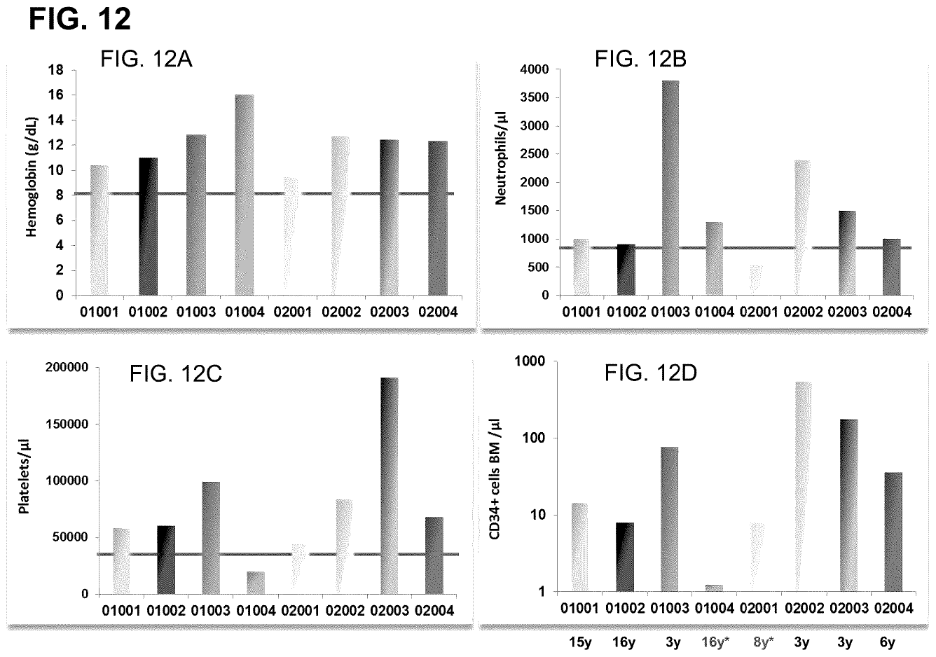

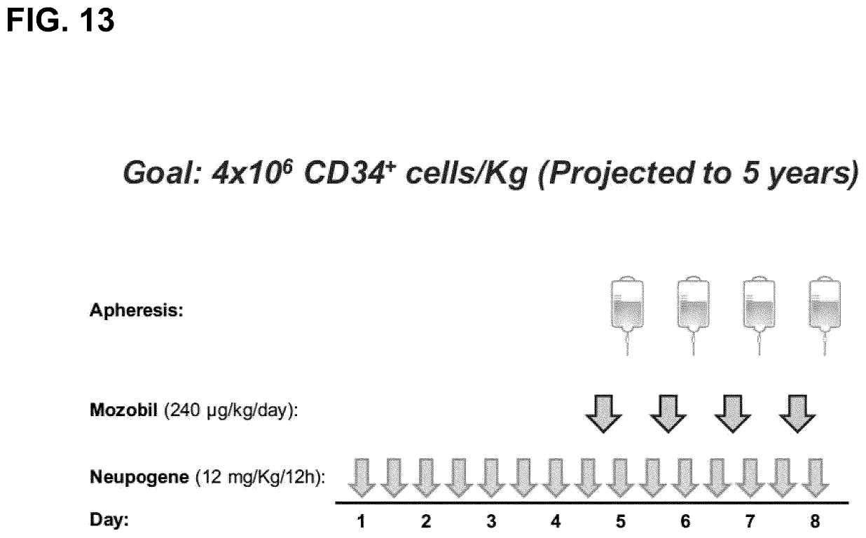

The main inclusion criteria for FANCOSTEM were Patients with a diagnosis of AF, confirmed by a test of chromosomal instability with diepoxybutane or mitomycin C, Age >1 year, and_At least one of the following parameters should be as high_as: 1) Hemoglobin: 8.0 g/dL, 2) Neutrophils: 750/mm³, 3) Platelets: 30,000/mm³. Ten patients were recruited, nine were screened out of which 2 failed (mosaic patients.) FIG. 12 shows the haematological parameters of recruited patients. Seven (7) patients were treated with G-CSF and Plerixafor. The mobilization regimen utilized the administration of G-CSF (neupogen; 12 μg/Kg/12 hours) and plerixafor (mozobil; 240 μg/kg body weight/day). Mobilized peripheral blood (mPB) CD34+ cells were transduced under GMP conditions with a PGK-FANCA-WPRE* LV using a short ex vivo transduction protocol (FIG. 13). While two patients who were 15 and 16 years old did not reach the threshold level of CD34+ cells in peripheral blood, apheresis could be conducted in five patients with ages between 3-5 years old. In these patients the median number of CD34+ cells/kg was of 6.6×10\hat{ }6 (range: 1.6×10\hat{ }6 to 7.6×10\hat{ }6). After CD34+ cell selection, the number of CD34+ cells/kg was 2.0×10\hat{ }6 (range: 8.5×10\hat{ }5 and 5.1×10\hat{ }6). No severe adverse events related to the mobilization regimen have been detected in any treated patient followed for up to 2.5 years.

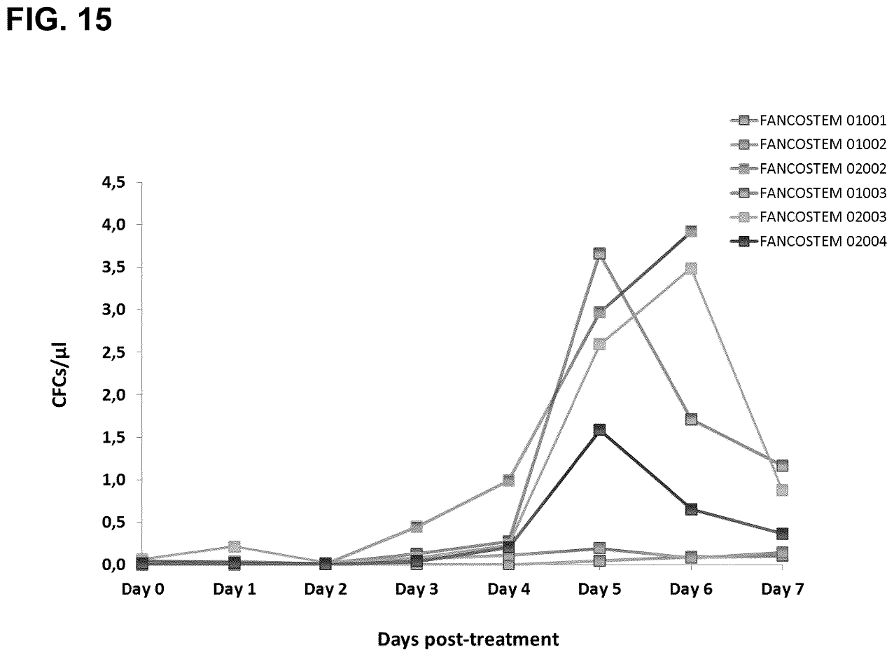

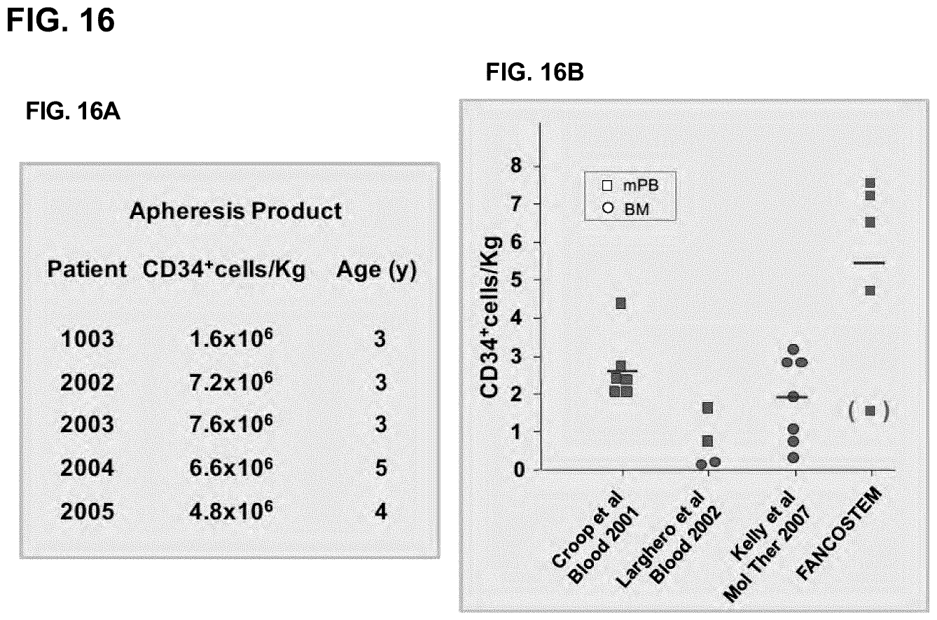

FIG. 14 shows G-SCF/Plerixafor-mediated Mobilization of CD34+ cells in FA-A patients and FIG. 15 shows G-SCF/Plerixafor-mediated Mobilization of colony forming cells (CFC) in FA-A patients. FIG. 16A shows CD34+ cell collection in FANCOSTEM and FIG. 16B shows compared to previous studies.

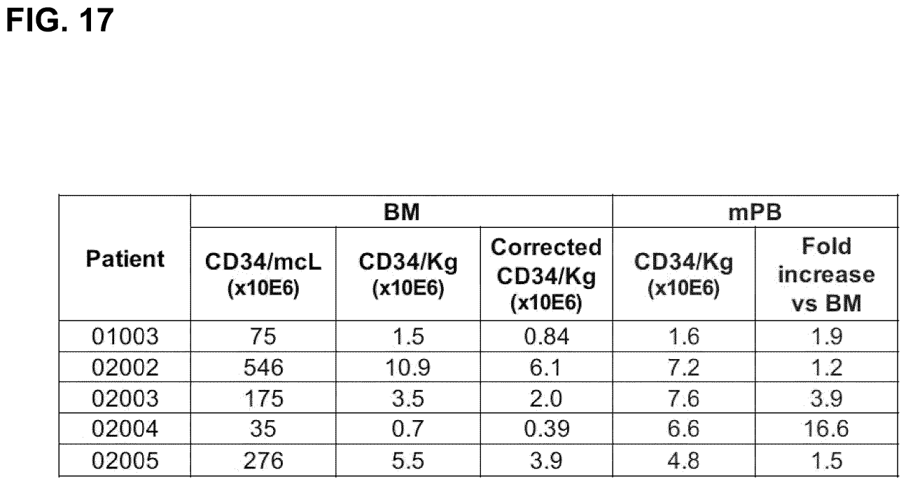

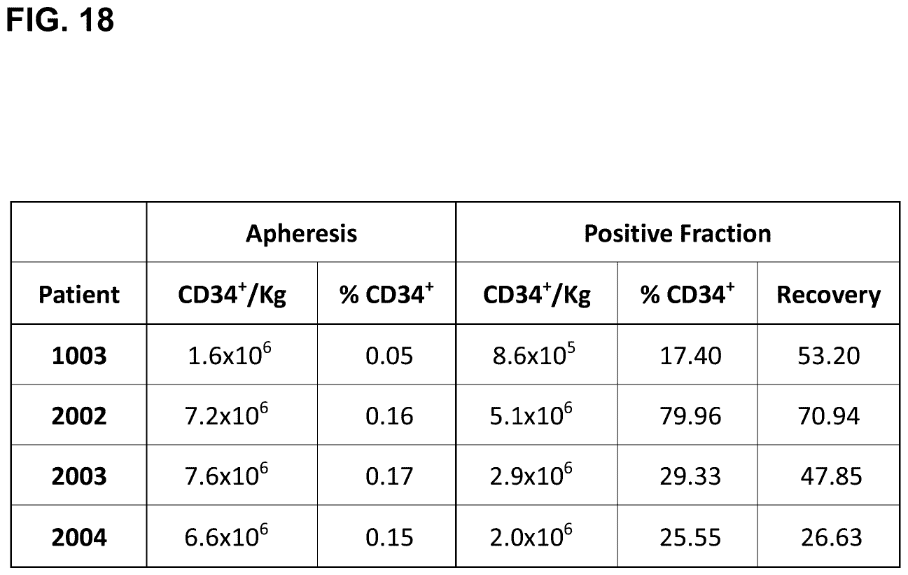

FIG. 17 shows comparison between predicted CD34+ cell numbers in bone marrow (BM) vs actual numbers in mobilized peripheral blood (mPB). FIG. 18 is a summary of the CD34⁺ cells collection in G-CSF/Plerixafor mobilized FA-A patients. The number of CD34+ after selection correlates with the number of CD34⁺ cells/μl in BM at day 0 (data not shown).

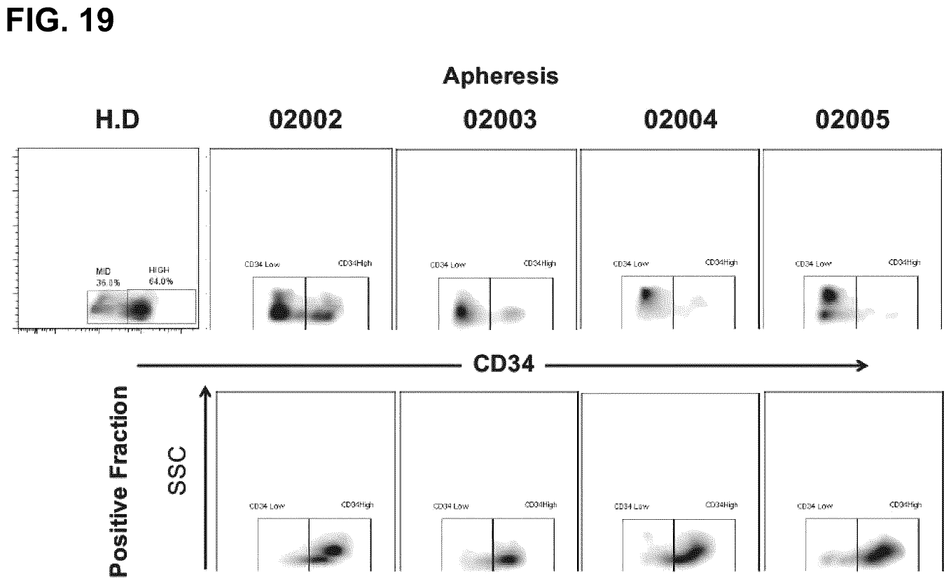

FIG. 19 depicts CD34 expression prior to and after immunoselection of mPB CD34+ cells from healthy donor (HD) and FA patients.

From these data, we concluded that compared to other clinical studies, evident improvements in the collection of CD34+ cells have been observed so far in patients treated with Filgrastim (G-CSF) and Plerixafor and only FA patients in early stages of the disease seem to be suitable for the collection of clinically relevant numbers of HSCs.

### Example 9. Clinical Study FANCOLEN

The second parallel clinical trial (FANCOLEN) aimed to evaluate the safety and efficacy of the gene therapy in patients with FA complementation group A (FA-A) (FANCOLEN). In order to restore the hematopoiesis of FA patients by the_infusion of gene corrected autologous HSCs, optimized vectors and transduction protocols were developed. Specifically, this was a Phase I/II clinical trial to evaluate the safety and efficacy of the infusion of_autologous CD34+ cells transduced with a lentiviral vector carrying the FANCA gene (Orphan drug) for patients with Fanconi Anemia Subtype A.

The main inclusion criteria were patients with a diagnosis of FA-A, Age >1 year, and at least one of the following parameters should be below the threshold of: 1) Hemoglobin: 8.0 g/dL; 2) Neutrophils: 1,000/mm³; 3) Platelets: 50,000/mm³.

More specific subject inclusion criteria include 1) Patients of the complementation group FA-A; 2) At least one of the following parameters must be lower than the values indicated: haemoglobin: 8.0 g/dL; neutrophils: 750/mm3; platelets: 30.000/mm3; 3) Minimum age: 1 year; 4) Maximum age: 21 years; 5) Lansky index >60% 6) Mild organ functional impairment; 7) Provide informed consent in accordance with current legislation; 8) Number of cells to transduce: at least 3×105 purified CD34+ cells/Kg of patient weight; 9) Women of childbearing age must have a negative urine pregnancy test at the baseline visit, and accept the use of an effective contraception method during participation in the trial.

Subject exclusion criteria include 1) Patients with an HLA-identical family donor; 2) Evidence of myelodysplastic syndrome or leukemia, or cytogenetic abnormalities predictive of these conditions in bone marrow aspirate analysis. This assessment should be made by valid studies two months before the patient enters the clinical trial; 3) Evidence that the patient has signs of somatic mosaicism with improved haematology; 4) Any concomitant disease or condition that, in the opinion of the investigator, deems the subject unfit to participate in the study; 5) Pre-existing sensory or motor impairment >=grade 2 according to the criteria of the National Cancer Institute (NCI); 6) Pregnant or breastfeeding women.

Route of administration: Patients received the cells transduced with therapeutic vector by intravenous infusion.

Dose of cells: The dose of cells patients received by transfusion was that which was obtained from transduction, between 3×105 and 4×106 purified CD34+ cells/kg of patient weight.

Below 3×105 CD34+ cells/Kg is highly unlikely to produce a patient graft from transduced cells, especially considering that this clinical trial will initially infuse unconditioned patients. In the gene therapy trial in Fanconi anemia patients conducted by Dr Williams (Kelly et al., 2007) the number of cells infused in 2 patients (FAAGT1001 and 1003) was 4.5×105 and 3.25×105 nucleated cells/kg of patient weight, respectively. In both patients, a transient improvement in Hb and platelet count was seen, but without being able to demonstrate an associated presence of the transgene. Unlike in Dr Williams' trial where cells were transduced for 4 days with a gamma-retroviral vector, in this clinical trial cells were transduced with a more efficacious lentiviral vector, and the transduction will be conducted for a maximum of 48 hours, i.e., much shorter than the 4 days used in the previous protocol. Given this, we considered 3×105 purified CD34+ cells/kg of patient body weight to be a reasonable lower limit for this trial.

The upper limit of 4×106 purified CD34+ cells/kg is not based on the need to limit the number of cells infused, as a greater number of cells increase the likelihood of graft. Rather the limit of 4×106 purified CD34+ cells/kg of patient body weight comes from the difficulty in mobilizing and collecting cells exceeding this number from patients with Fanconi anemia, characterized by having a reduced CD34+ cell count in their bone marrow (Jacome et al., 2009).

Dosage regimen: The cells transduced with the therapeutic vector were infused in a single dose to the patient. This was for two main reasons: 1) All hematopoietic gene therapy trials conducted so far have been performed using a single infusion of transduced cells (Naldini, 2011). Following this prior experience, we were not inclined to vary this parameter. 2) Single infusion of all transduced cells would increase the likelihood that there is a greater graft, compared to infusion of the same dose fractionated.

Recruitment period: Patients are recruited over a period of 3 years. As patients with subtype FA-A are the most frequent in the FA patient population (around 80% of Spanish patients with FA correspond to this complementation group (Casado et al. (2007)), the constructed therapeutic vector carries the FANCA gene. Therefore, of FA patients only those patients belonging to subtype FA-A can participate in this study. Any patients in this complementation group, pediatric or adult, provided they meet the defined inclusion and exclusion criteria, were included in the study.

Specific description of the primary and, if any, secondary variables that will be assessed in the clinical trial include 1) Main variable: A) To determine the toxicity associated with infusion of autologous CD34+ cells transduced with the therapeutic lentiviral vector in patients with Fanconi anemia subtype A. B) To determine the degree of grafting associated with the infusion of autologous CD34+ cells transduced with the therapeutic lentiviral vector in patients with Fanconi anemia subtype A 2) Secondary variables: To determine the clinical response associated with infusion of autologous CD34+ cells transduced with the therapeutic lentiviral vector in patients with Fanconi anemia subtype A.

CD34+ cells from bone marrow and/or mobilized in peripheral blood (fresh and/or cryopreserved) from patients with Fanconi anemia subtype A (FA-A) were transduced ex vivo with a lentiviral vector carrying the FANCA gene (orphan drug) (FIG. 11). After cell transduction, patients received an infusion of these genetically corrected stem cells in order to restore hematopoiesis.

Assessment: Patients were assessed before initiating treatment, obtaining prior informed consent. The assessment was carried out in the month before infusion of genetically corrected cells, through a standard physical examination (including weight and height), peripheral blood cell counts, basic biochemistry, and a bone marrow aspirate.

Transduction and infusion of genetically corrected CD34+ cells: The purified CD34+ cell population was transduced ex vivo with the therapeutic lentiviral vector. After cell transduction, product quality control evaluations was carried out, aliquots were cryopreserved for further study, and the product was prepared for infusion into patients.

If in two patients infused with an acceptable number of transduced cells (at least 1 million cells infused/kg of body weight, in an aliquot in which at least 0.3 copies of the vector/cell is detected after at least 7 days in culture in vitro) AT LEAST 0.1 COPIES VECTOR/CELL is not observed in either the bone marrow or peripheral blood at 6 MONTHS POST-INFUSION, the subsequent patients will be subject to conditioning process prior to cell infusion.

For a patient to be eligible for conditioning of any kind there must be a suitable method of rescuing potential aplasia of bone marrow associated with conditioning and possible implant failure of transduced cells. Rescue methods include a unit of umbilical cord blood or hematopoietic cells from a haploidentical donor. Cells will be infused intravenously.

The dose of cells patients receive by infusion was that which is obtained from the transduction process, between 3×10⁵ and 4×10⁶ CD34⁺ cells/Kg of patient weight.

Cells are infused into the patient a single dose.

Transduced cells are infused immediately after the transduction process is completed.

The product infused consists of a suspension of CD34⁺ cells which was packaged in a sterile bag for infusion by the CLINISTEM GMP laboratory at CIEMAT.

Recruitment Period: 3 years from infusion of the 1st patient.

Follow-up period: two years from the infusion of transduced cells. However, patients are monitored outside the clinical trial for a period of 10 years.

Monitoring of the graft of transduced cells will be carried out on peripheral blood and bone marrow samples.

First 72 hours after infusion: During this period, vital signs were recorded every 8 hours and vital organ functions were monitored (electrolyte profile, haematology, renal and hepatic function) every 24 hours.

Subsequently the following checks were carried out as shown in Table 2.

TABLE 2

| | Peripheral blood | | | | | | | | | | | | |
|---|---|---|---|---|---|---|---|---|---|---|---|---|---|
| | Week: | | | | Month: | | | | | | | | |
| Haematological | 2 | 4 | 6 | — | 2 | 4 | 6 | 9 | 12 | 15 | 18 | 21 | 24 |
| monitoring | | | | | | | | | | | | | |
| Copies of the | 2 | 4 | 6 | — | 2 | 4 | 6 | 9 | 12 | 15 | 18 | 21 | 24 |
| therapeutic vector | | | | | | | | | | | | | |
| | Bone marrow | | | | | | | | | | | | |
| | Week: | | | | Month: | | | | | | | | |
| Cytology | | 4 | | — | | | 6 | | 12 | | | | 24 |
| Copies of the | | 4 | | — | | | 6 | | 12 | | | | 24 |
| therapeutic vector | | | | | | | | | | | | | |

Patients diagnosed with FA and belonging to complementation group FA-A will be included in the study. Patients were considered if cellular phenotype correction has been demonstrated by transduction with vectors carrying the FANCA gene or if bi-allelic pathogenic mutations in this gene are demonstrated.

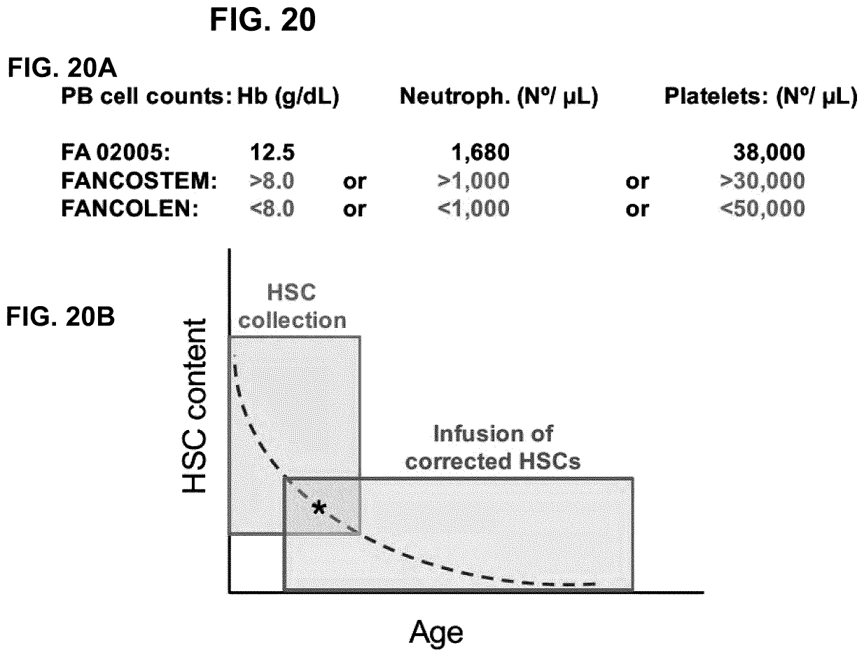

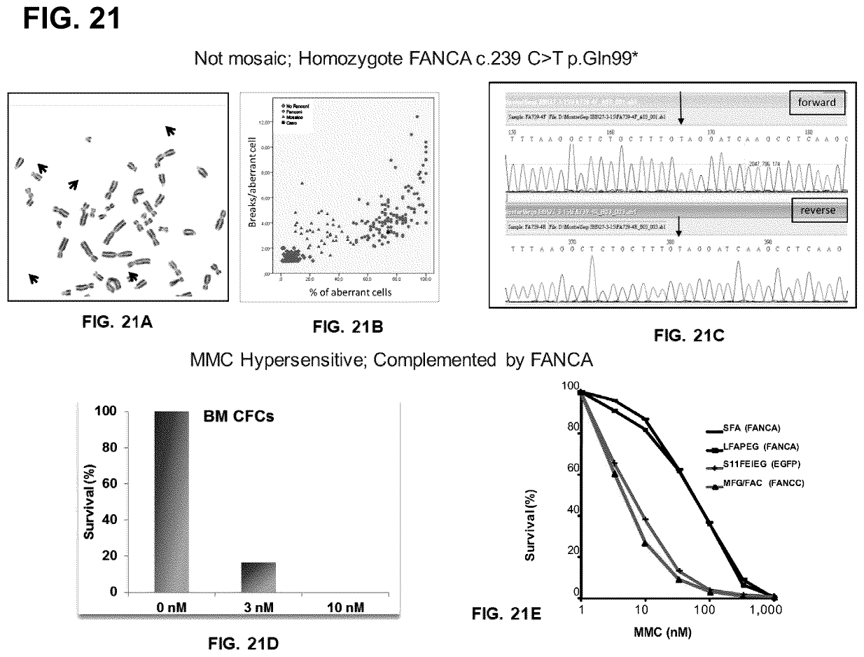

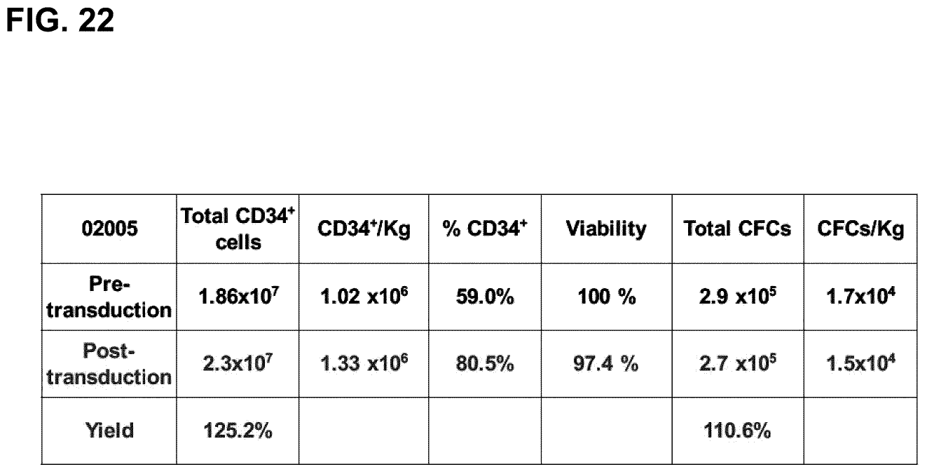

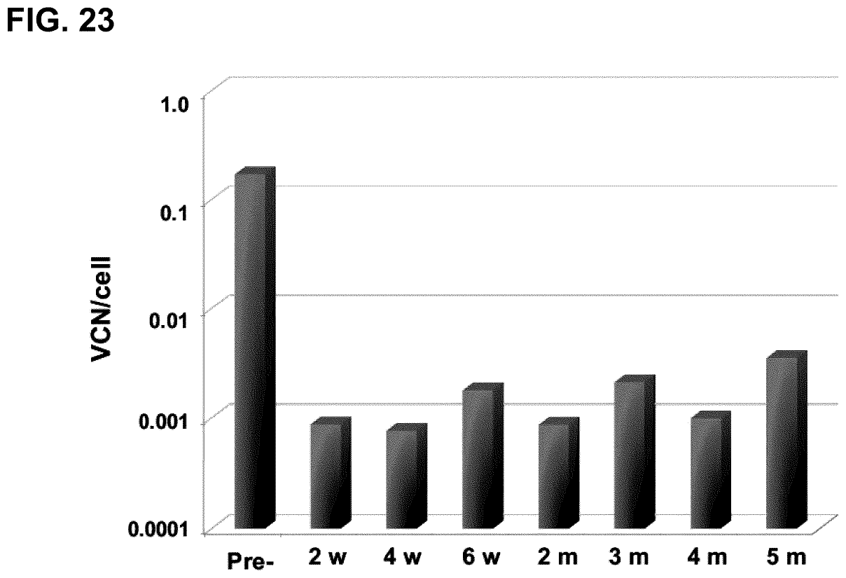

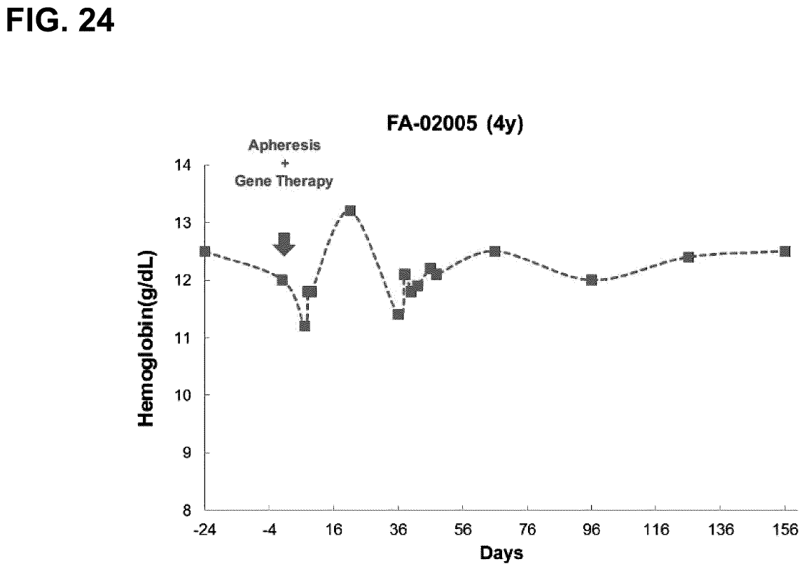

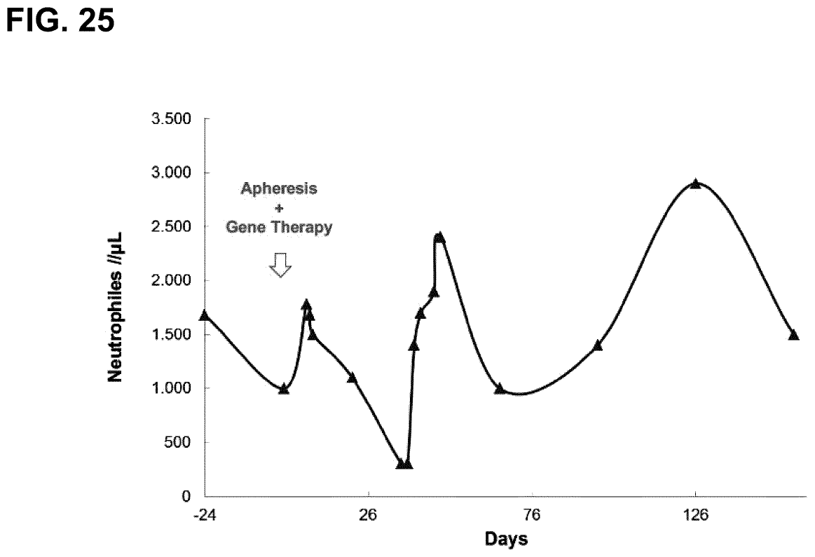

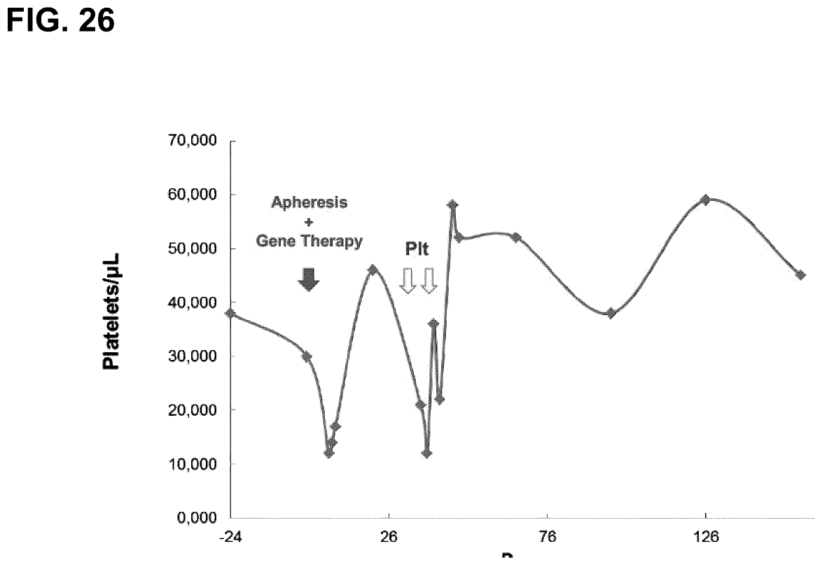

Patient FA 02005 fit the criteria for FANCOSTEM and FANCOLEN as summarized in FIG. 20. FIG. 21 depicts the tests showing FA diagnosis of patient FA-02005 prior to gene therapy and FIG. 22 shows the follow up of the cell manufacturing process for patient FA-02005. FIG. 23 shows vector copy number in patient FA02005 prior to gene therapy and 2 weeks, 4 weeks, 6 weeks, 2 months, 3 months, 4 months, and 5 months after gene therapy. Follow-up of the first not-conditioned FA-A patient prior to and after gene therapy, patient FA02005 (4 year old) is represented for hemoglobin (FIG. 24), neutrophils (FIG. 25), and platelets (FIG. 26).

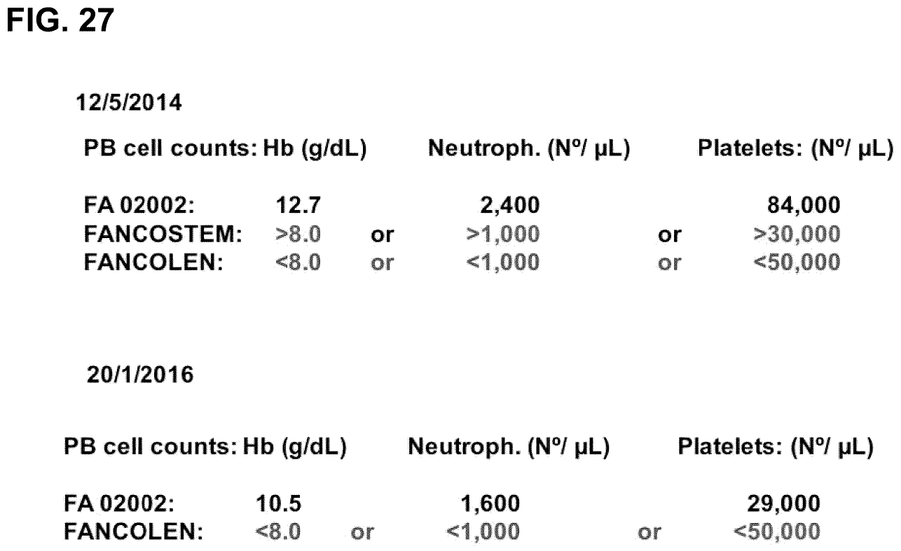

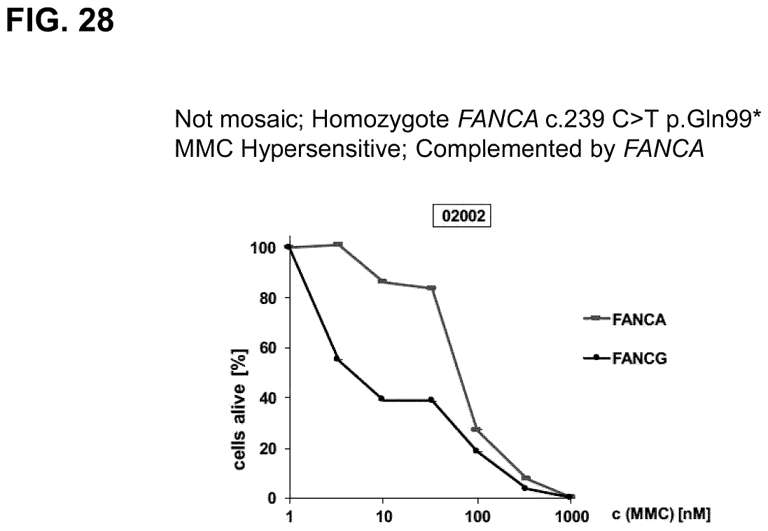

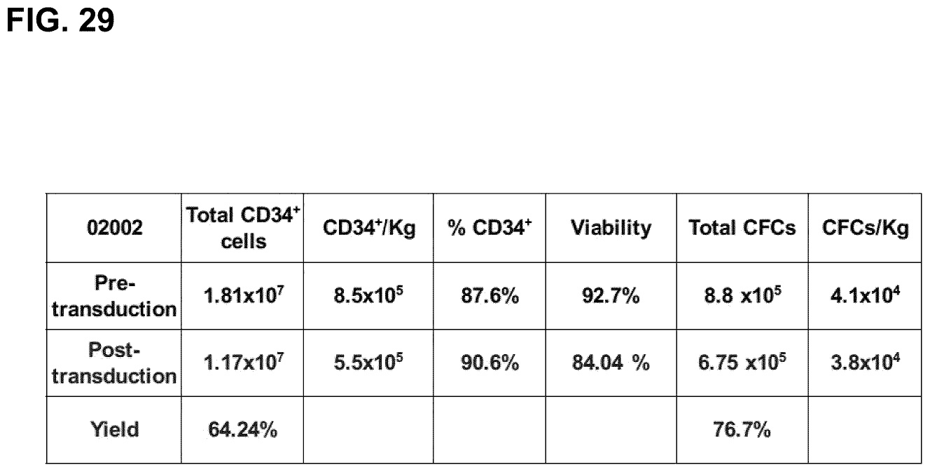

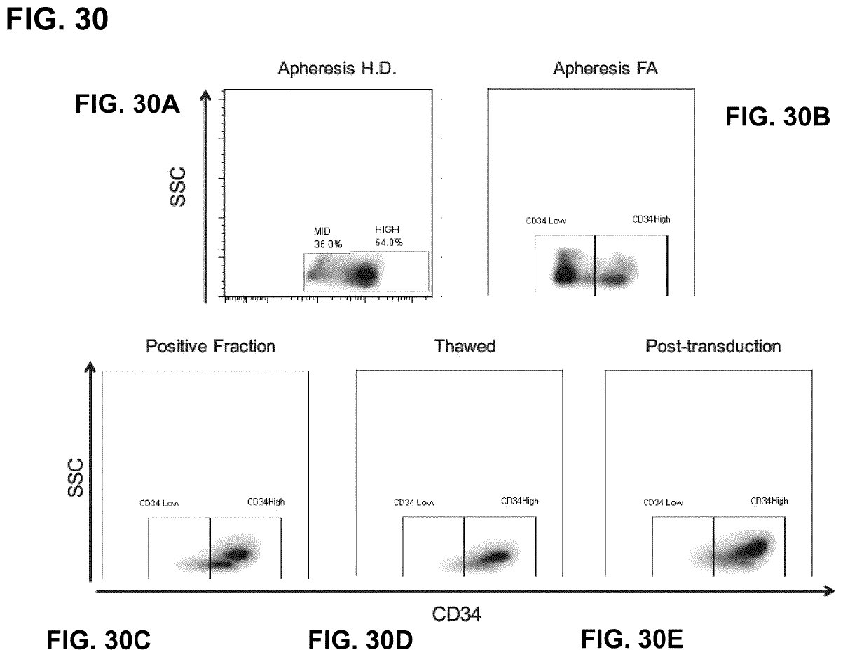

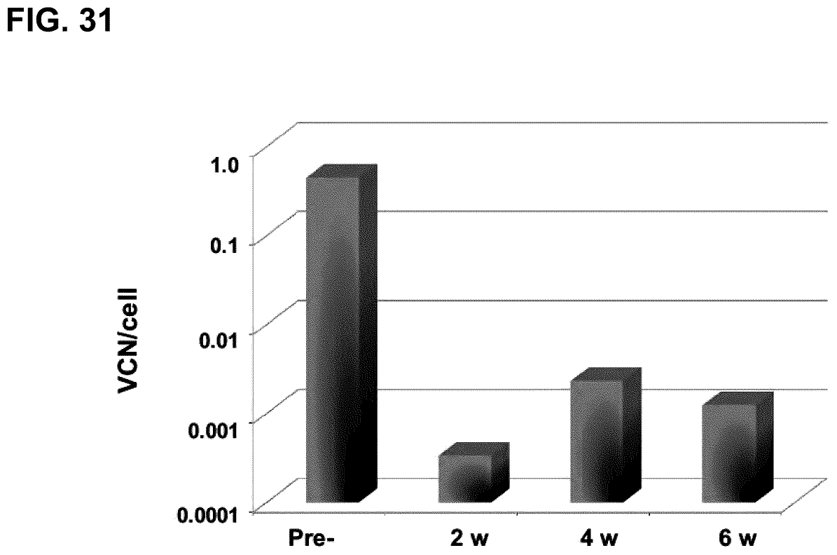

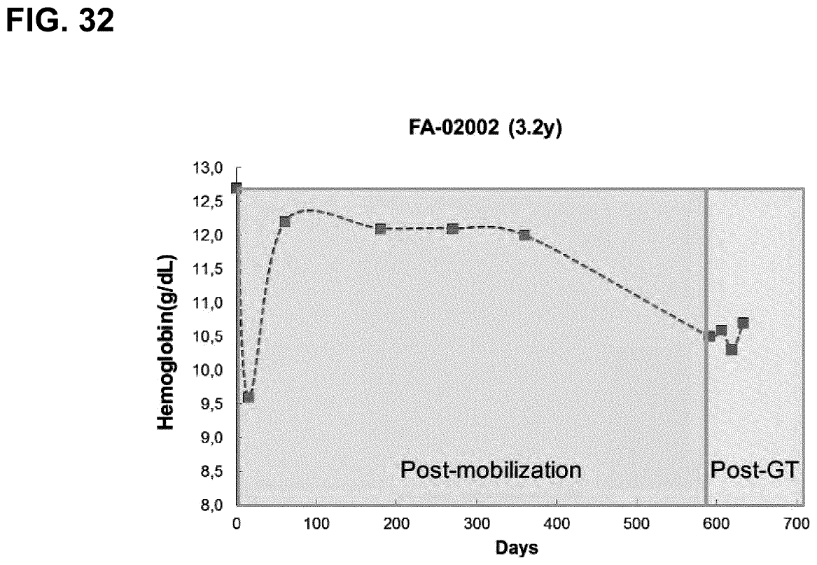

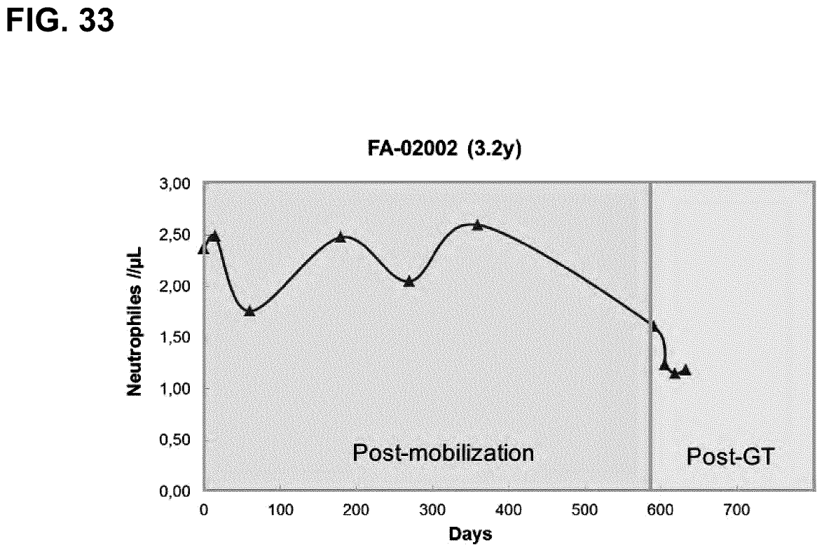

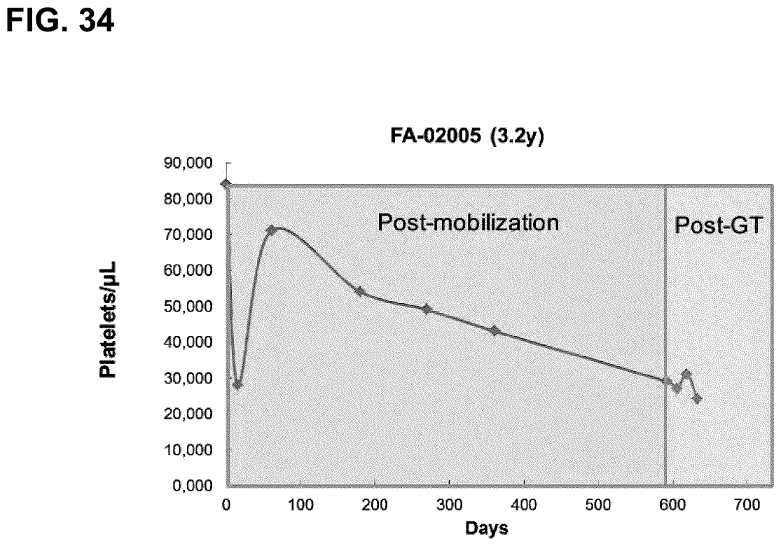

For patient FA02002, the hematological evolution is presented in FIG. 27, diagnosis is presented in FIG. 28, and follow up of the cell manufacturing process is presented in FIG. 29. FIG. 30 shows analysis of CD34 expression in healthy donor and FA mPB during the different steps required for LV-transduction for patient FA-02002. FIG. 31 shows vector copy number in patient FA02002 prior to gene therapy and 2 weeks, 4 weeks, 6 weeks after gene therapy. Follow-up of the patient FA02002 (infused with cryopreserved cells) is presented for hemoglobin (FIG. 32), neutrophils (FIG. 33), and platelets (FIG. 34).

The data for these two patients support the conclusion that the patients were infused with a significant number of gene-corrected mobilized peripheral blood (mPB) CD34+ cells. No serious adverse events have been observed in any of the two treated patients. Gene marking levels of around 1-5 copies/1000 peripheral blood cells were detected in treated patients at 15 days to 5 months post-GT, with moderate increases along time. These viral copy numbers were around 100× higher compared to the highest value detected at 3 weeks post-GT in previous trials (3 copies/10\hat{ }5 cells; Kelly et al. 2007).

### Example 10. Transduction of Fresh mPB CD34⁺ Cells from FA-A Patients

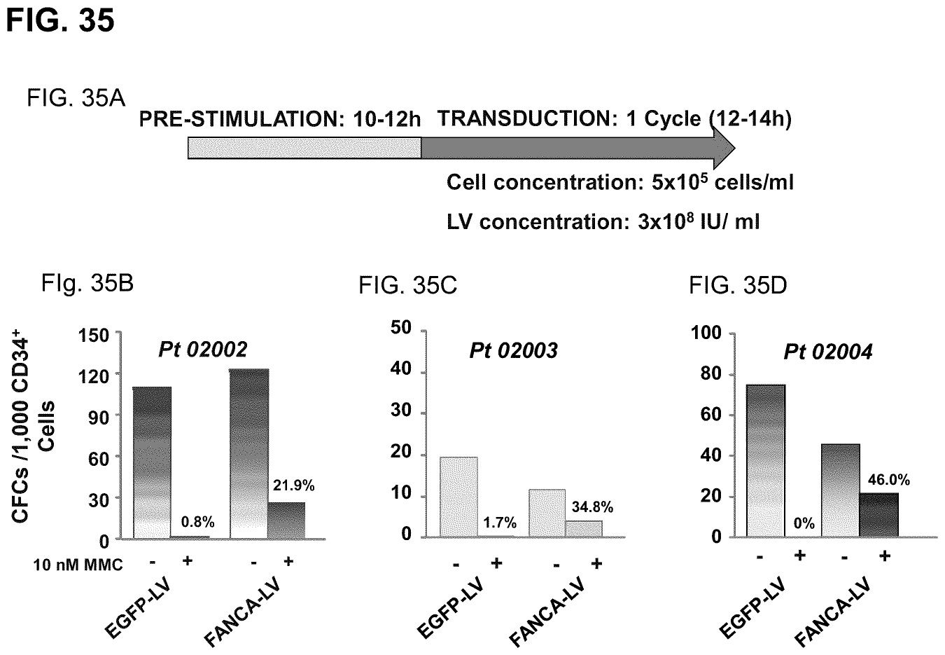

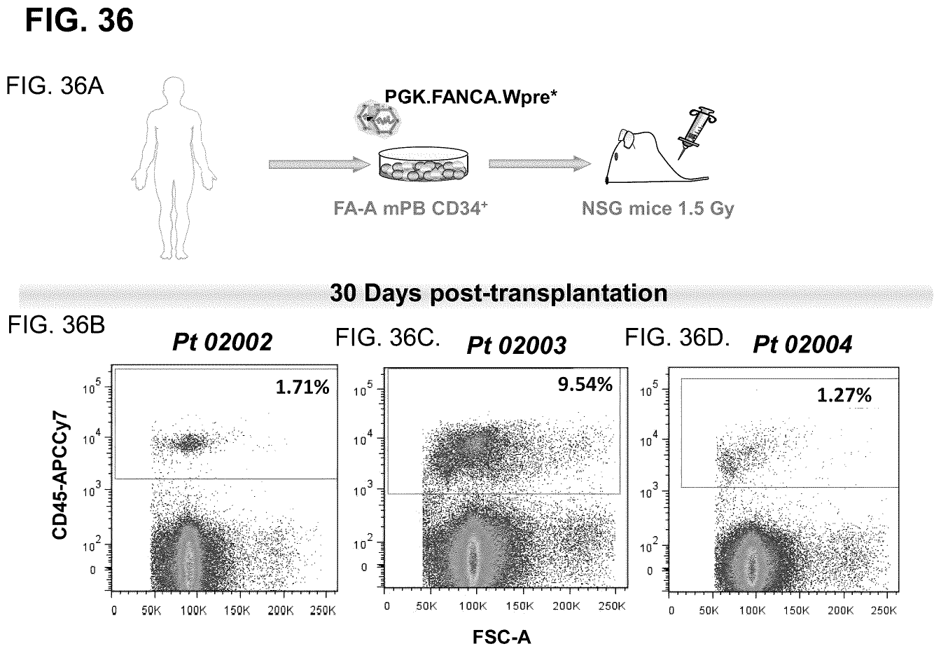

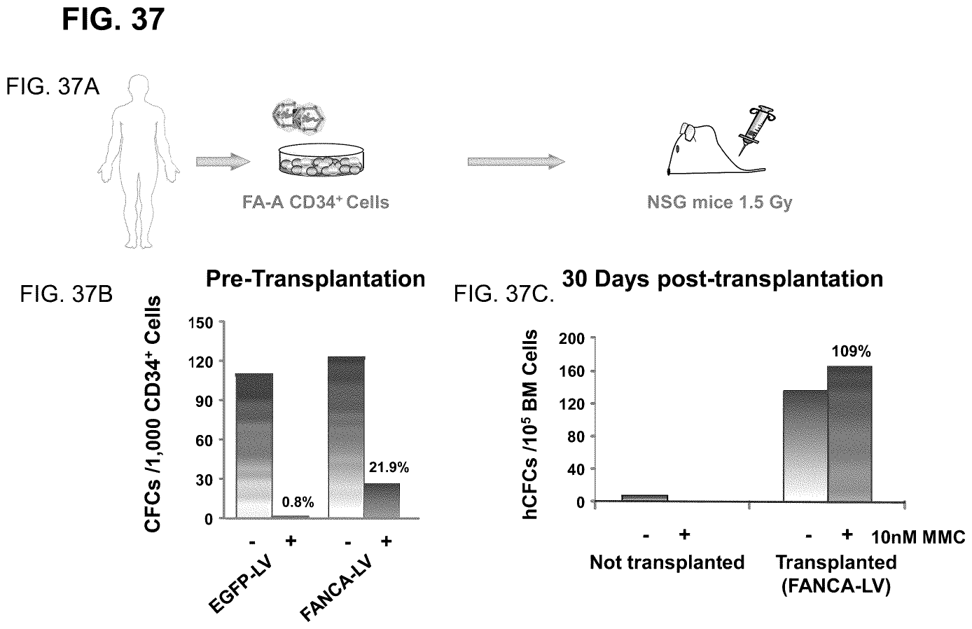

The short transduction with the therapeutic vector of small aliquots of mobilized peripheral blood (mPB) CD34+ samples from patients treated with the G-CSF/Plerixafor regimen showed transduction efficacies between 17-45% (FIG. 35). Small aliquots of these samples were transplanted into NSG mice conditioned with 1.5 Gy. Most of the transplanted samples engrafted into the NSG mice (1-10% of the BM cells were hCD45+/mCD45−) (FIG. 36). Moreover, an evident selection advantage of corrected CD34+ FA-A cells was observed in engrafted mice (FIG. 37).

These results show that transduced FA-A mPB CD34⁺ cells engraft into NSG mice and there is an in vivo proliferation advantage of corrected human FA-A repopulating cells takes place in NSG recipient mice.

1. (canceled)

2. An expression cassette comprising a polynucleotide sequence comprising in the following 5′ to 3′ order:
(a) a human phosphoglycerate kinase (PGK) promoter sequence or a functional homolog or variant thereof;
(b) a sequence encoding a human FANC polypeptide or a functional fragment or variant thereof;
(c) a woodchuck hepatitis virus regulatory element (WPRE) sequence or a functional variant or fragment thereof,
wherein the sequence encoding the human FANC polypeptide or functional fragment or variant thereof is operably linked to the PGK promoter sequence; and
wherein the WPRE element comprises a sequence having at least 90% identity to SEQ ID NO: 23.

3. The expression cassette of claim 2, wherein the FANC polypeptide is selected from FANCA, FANCC, and FANCG.

4. The expression cassette of claim 3, wherein the FANCA polypeptide comprises a sequence having at least 90% identity to SEQ ID NO: 25.

5. The expression cassette of claim 3, wherein the FANCA polypeptide comprises the sequence of SEQ ID NO: 25.

6. The expression cassette of claim 3, wherein the sequence encoding the FANCA polypeptide comprises a sequence having at least 90% identity to SEQ ID NO: 8.

7. The expression cassette of claim 3, wherein the sequence encoding the FANCA polypeptide comprises the sequence of SEQ ID NO: 8.

8. The expression cassette of claim 2, wherein the PGK promoter comprises a sequence having at least 90% identity to SEQ ID NO: 7.

9. The expression cassette of claim 2, wherein the PGK promoter comprises the sequence of SEQ ID NO: 7.

10. The expression cassette of claim 2, wherein the WPRE element comprises the sequence of SEQ ID NO: 23.

11. The expression cassette of claim 2, wherein the cassette comprises a sequence having at least 90% identity to nucleotides 3541 to 9178 of SEQ ID NO: 24.

12. The expression cassette of claim 2, wherein the cassette comprises the sequence of nucleotides 3541 to 9178 of SEQ ID NO: 24

13. The expression cassette of claim 2, further comprising one or more enhancer sequences.

14. The expression cassette of claim 2, further comprising:
(d) a polypurine tract (PPT) or polyadenylation (polyA) signal sequence.

15. The expression cassette of claim 2, further comprising one or more of the following sequences:
(e) a packaging signal sequence;
(f) a truncated Gag sequence;
(g) a Rev responsive element (RRE);
(h) a central polypurine tract (cPPT); and
(i) a central terminal sequence (CTS).

16. A recombinant gene delivery vector comprising the expression cassette of claim 2.

17. The recombinant gene delivery vector of claim 16, wherein the recombinant gene delivery vector is a virus or viral vector.

18. The recombinant gene delivery vector of claim 17, wherein the virus or viral vector is a lentivirus (LV).

19. A cell comprising the expression cassette of claim 2.

20. The cell of claim 19, wherein the cell is a hematopoietic stem cell.

21. The cell of claim 19, wherein the cell is a CD34⁺ cell.

22. A pharmaceutical composition comprising a pharmaceutically acceptable excipient and the recombinant gene delivery vector of claim 16.

23. A pharmaceutical composition comprising a pharmaceutically acceptable excipient and the cell of claim 19.

24. A method for treating Fanconi anemia in a subject in need thereof, comprising
(a) providing to the subject a combination of: (i) G-CSF or Filgrastim; and (ii) Plerixafor to mobilize CD34+ cells within the subject;
(b) obtaining a biological sample comprising CD34+ cells from the subject;
(c) preparing a cell population enriched for CD34+ cells from the biological sample; and
(d) transducing the cell population enriched for CD34+ cells with a lentiviral vector, wherein the lentiviral vector comprises the expression cassette of claim 2.

25. The method of claim 24, wherein the biological sample is peripheral blood or bone marrow.

26. The method of claim 24, wherein the subject is a non-conditioned subject.

27. The method of claim 24, comprising administering to the subject the cell population at a dose of at least 3×10⁵ cells/kg of the subject's weight.

28. The method of claim 24, comprising administering to the subject the cell population at a dose of at most 4×10⁶ cells/kg of the subject's weight.


SEQUENCE LISTING (25 sequences)

SEQ ID NO: 1 - DNA, 234 bp, Human immunodeficiency virus 1
  source 1..234: mol_type = unassigned DNA
aggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcct

SEQ ID NO: 2 - DNA, 126 bp, Human immunodeficiency virus 1
  source 1..126: mol_type = unassigned DNA
ctctctcgacgcaggactcggcttgctgaagcgcgcacggcaagaggcgaggggcggcgactggtgagtacgccaaaaattttgactagcggaggctagaaggagagagatgggtgcgagagcgtc

SEQ ID NO: 3 - DNA, 181 bp, Human immunodeficiency virus 1
  source 1..181: mol_type = unassigned DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 4 - DNA, 234 bp, Human immunodeficiency virus 1
  source 1..234: mol_type = unassigned DNA
tggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagca

SEQ ID NO: 5 - DNA, 204 bp, unidentified
  source 1..204: mol_type = unassigned DNA; note = Human herpesvirus-5
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 6 - DNA, 118 bp, Human immunodeficiency virus 1
  source 1..118: mol_type = unassigned DNA
ttttaaaagaaaaggggggattggggggtacagtgcaggggaaagaatagtagacataatagcaacagacatacaaactaaagaattacaaaaacaaattacaaaaattcaaaatttt

SEQ ID NO: 7 - DNA, 511 bp, Homo sapiens
  source 1..511: mol_type = unassigned DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 8 - DNA, 4368 bp, Homo sapiens
  source 1..4368: mol_type = unassigned DNA
atgtccgactcgtgggtcccgaactccgcctcgggccaggacccagggggccgccggagggcctgggccgagctgctggcgggaagggtcaagagggaaaaatataatcctgaaagggcacagaaattaaaggaatcagctgtgcgcctcctgcgaagccatcaggacctgaatgcccttttgcttgaggtagaaggtccactgtgtaaaaaattgtctctcagcaaagtgattgactgtgacagttctgaggcctatgctaatcattctagttcatttataggctctgctttgcaggatcaagcctcaaggctgggggttcccgtgggtattctctcagccgggatggttgcctctagcgtgggacagatctgcacggctccagcggagaccagtcaccctgtgctgctgactgtggagcagagaaagaagctgtcttccctgttagagtttgctcagtatttattggcacacagtatgttctcccgtctttccttctgtcaagaattatggaaaatacagagttctttgttgcttgaagcggtgtggcatcttcacgtacaaggcattgtgagcctgcaagagctgctggaaagccatcccgacatgcatgctgtgggatcgtggctcttcaggaatctgtgctgcctttgtgaacagatggaagcatcctgccagcatgctgacgtcgccagggccatgctttctgattttgttcaaatgtttgttttgaggggatttcagaaaaactcagatctgagaagaactgtggagcctgaaaaaatgccgcaggtcacggttgatgtactgcagagaatgctgatttttgcacttgacgctttggctgctggagtacaggaggagtcctccactcacaagatcgtgaggtgctggttcggagtgttcagtggacacacgcttggcagtgtaatttccacagatcctctgaagaggttcttcagtcataccctgactcagatactcactcacagccctgtgctgaaagcatctgatgctgttcagatgcagagagagtggagctttgcgcggacacaccctctgctcacctcactgtaccgcaggctctttgtgatgctgagtgcagaggagttggttggccatttgcaagaagttctggaaacgcaggaggttcactggcagagagtgctctcctttgtgtctgccctggttgtctgctttccagaagcgcagcagctgcttgaagactgggtggcgcgtttgatggcccaggcattcgagagctgccagctggacagcatggtcactgcgttcctggttgtgcgccaggcagcactggagggcccctctgcgttcctgtcatatgcagactggttcaaggcctcctttgggagcacacgaggctaccatggctgcagcaagaaggccctggtcttcctgtttacgttcttgtcagaactcgtgccttttgagtctccccggtacctgcaggtgcacattctccacccacccctggttcccagcaagtaccgctccctcctcacagactacatctcattggccaagacacggctggccgacctcaaggtttctatagaaaacatgggactctacgaggatttgtcatcagctggggacattactgagccccacagccaagctcttcaggatgttgaaaaggccatcatggtgtttgagcatacggggaacatcccagtcaccgtcatggaggccagcatattcaggaggccttactacgtgtcccacttcctccccgccctgctcacacctcgagtgctccccaaagtccctgactcccgtgtggcgtttatagagtctctgaagagagcagataaaatccccccatctctgtactccacctactgccaggcctgctctgctgctgaagagaagccagaagatgcagccctgggagtgagggcagaacccaactctgctgaggagcccctgggacagctcacagctgcactgggagagctgagagcctccatgacagaccccagccagcgtgatgttatatcggcacaggtggcagtgatttctgaaagactgagggctgtcctgggccacaatgaggatgacagcagcgttgagatatcaaagattcagctcagcatcaacacgccgagactggagccacgggaacacattgctgtggacctcctgctgacgtctttctgtcagaacctgatggctgcctccagtgtcgctcccccggagaggcagggtccctgggctgccctcttcgtgaggaccatgtgtggacgtgtgctccctgcagtgctcacccggctctgccagctgctccgtcaccagggcccgagcctgagtgccccacatgtgctggggttggctgccctggccgtgcacctgggtgagtccaggtctgcgctcccagaggtggatgtgggtcctcctgcacctggtgctggccttcctgtccctgcgctctttgacagcctcctgacctgtaggacgagggattccttgttcttctgcctgaaattttgtacagcagcaatttcttactctctctgcaagttttcttcccagtcacgagatactttgtgcagctgcttatctccaggccttattaaaaagtttcagttcctcatgttcagattgttctcagaggcccgacagcctctttctgaggaggacgtagccagcctttcctggagacccttgcaccttccttctgcagactggcagagagctgccctctctctctggacacacagaaccttccgagaggtgttgaaagaggaagatgttcacttaacttaccaagactggttacacctggagctggaaattcaacctgaagctgatgctctttcagatactgaacggcaggacttccaccagtgggcgatccatgagcactttctccctgagtcctcggcttcagggggctgtgacggagacctgcaggctgcgtgtaccattcttgtcaacgcactgatggatttccaccaaagctcaaggagttatgaccactcagaaaattctgatttggtctttggtggccgcacaggaaatgaggatattatttccagattgcaggagatggtagctgacctggagctgcagcaagacctcatagtgcctctcggccacaccccttcccaggagcacttcctctttgagattttccgcagacggctccaggctctgacaagcgggtggagcgtggctgccagccttcagagacagagggagctgctaatgtacaaacggatcctcctccgcctgccttcgtctgtcctctgcggcagcagcttccaggcagaacagcccatcactgccagatgcgagcagttcttccacttggtcaactctgagatgagaaacttctgctcccacggaggtgccctgacacaggacatcactgcccacttcttcaggggcctcctgaacgcctgtctgcggagcagagacccctccctgatggtcgacttcatactggccaagtgccagacgaaatgccccttaattttgacctctgctctggtgtggtggccgagcctggagcctgtgctgctctgccggtggaggagacactgccagagcccgctgccccgggaactgcagaagctacaagaaggccggcagtttgccagcgatttcctctcccctgaggctgcctccccagcacccaacccggactggctctcagctgctgcactgcactttgcgattcaacaagtcagggaagaaaacatcaggaagcagctaaagaagctggactgcgagagagaggagctattggttttccttttcttcttctccttgatgggcctgctgtcgtcacatctgacctcaaatagcaccacagacctgccaaaggctttccacgtttgtgcagcaatcctcgagtgtttagagaagaggaagatatcctggctggcactctttcagttgacagagagtgacctcaggctggggcggctcctcctccgtgtggccccggatcagcacaccaggctgctgcctttcgctttttacagtcttctctcctacttccatgaagacgcggccatcagggaagaggccttcctgcatgttgctgtggacatgtacttgaagctggtccagctcttcgtggctggggatacaagcacagtttcacctccagctggcaggagcctggagctcaagggtcagggcaaccccgtggaactgataacaaaagctcgtctttttctgctgcagttaatacctcggtgcccgaaaaagagcttctcacacgtggcagagctgctggctgatcgtggggactgcgacccagaggtgagcgccgccctccagagcagacagcaggctgcccctgacgctgacctgtcccaggagcctcatctcttctga

SEQ ID NO: 9 - DNA, 380 bp, unidentified
  source 1..380: mol_type = unassigned DNA; note = Human herpesvirus 5
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 10 - DNA, 122 bp, unidentified
  source 1..122: mol_type = unassigned DNA; note = Simian virus 40
aacttgtttattgcagcttataatggttacaaataaagcaatagcatcacaaatttcacaaataaagcatttttttcactgcattctagttgtggtttgtccaaactcatcaatgtatctta

SEQ ID NO: 11 - DNA, 136 bp, unidentified
  source 1..136: mol_type = unassigned DNA; note = Simian virus 40
atcccgcccctaactccgcccagttccgcccattctccgccccatggctgactaattttttttatttatgcagaggccgaggccgcctcggcctctgagctattccagaagtagtgaggaggcttttttggaggcc

SEQ ID NO: 12 - DNA, 28 bp, Human immunodeficiency virus 1
  source 1..28: mol_type = unassigned DNA
tttaaaagaaaaggggggattggggggt

SEQ ID NO: 13 - DNA, 83 bp, Human immunodeficiency virus 1
  source 1..83: mol_type = unassigned DNA
gaattcgagctcggtacctttaagaccaatgacttacaaggcagctgtagatcttagccactttttaaaagaaaaggggggac

SEQ ID NO: 14 - DNA, 795 bp, Escherichia coli
  source 1..795: mol_type = unassigned DNA
atgattgaacaagatggattgcacgcaggttctccggcggcttgggtggagaggctattcggctatgactgggcacaacagacaatcggctgctctgatgccgccgtgttccggctgtcagcgcaggggcgtccggttctttttgtcaagaccgacctgtccggtgccctgaatgaactgcaagacgaggcagcgcggctatcgtggctggcgacgacgggcgttccttgcgcggctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgtctatgcccgacggcgaggatctcgtcgtgacccacggcgatgcctgcttgccgaatatcatggtggaaaatggccgcttttctggattcatcgactgtggccgtctgggtgtggcggaccgctatcaggacatagcgttggctacccgtgatattgctgaagagcttggcggcgaatgggctgaccgcttccttgtgctttacggtatcgccgcgcccgattcgcagcgcatcgccttctatcgccttcttgacgagttcttctga

SEQ ID NO: 15 - DNA, 137 bp, Escherichia coli
  source 1..137: mol_type = unassigned DNA
gcattggcgcagaaaaaaatgcctgatgcgacgctgcgcgtcttatactcccacatatgccagattcagcaacggatacggcttccccaacttgcccacttccatacgtgtcctccttaccagaaatttatccttaa

SEQ ID NO: 16 - DNA, 589 bp, unidentified
  misc_feature 1..589: note = ColE1/pMB1/pBR322/pUC origin of replication plasmid sequence
  source 1..589: mol_type = unassigned DNA
ttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaa

SEQ ID NO: 17 - DNA, 22 bp, Escherichia coli
  source 1..22: mol_type = unassigned DNA
taatgtgagttagctcactcat

SEQ ID NO: 18 - DNA, 31 bp, Escherichia coli
  source 1..31: mol_type = unassigned DNA
tttacactttatgcttccggctcgtatgttg

SEQ ID NO: 19 - DNA, 17 bp, Escherichia coli
  source 1..17: mol_type = unassigned DNA
ttgtgagcggataacaa

SEQ ID NO: 20 - DNA, 19 bp, unidentified
  source 1..19: mol_type = unassigned DNA; note = Bacteriophage T3
aattaaccctcactaaagg

SEQ ID NO: 21 - DNA, 19 bp, unidentified
  source 1..19: mol_type = unassigned DNA; note = Bacteriophage T7
cctatagtgagtcgtatta

SEQ ID NO: 22 - DNA, 428 bp, unidentified
  source 1..428: mol_type = unassigned DNA; note = F1 bacteriophage
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaat

SEQ ID NO: 23 - DNA, 677 bp, Woodchuck hepatitis virus
  source 1..677: mol_type = unassigned DNA
cgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctgctgccggctctgcggcctcttccgcgtcttcgccttcgccctcagacgagtcggatctccctttgggccgcctccccgcctg

SEQ ID NO: 24 - DNA, 11433 bp, synthetic construct
  misc_feature 1..11433: note = Made in lab transfer cassette pCCL-SIN-cPPT/CTS -hPGK-hFANCA-WPRE
  source 1..11433: mol_type = other DNA
catgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatcgaattccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatcccccgggctgcaggaattcatgtccgactcgtgggtcccgaactccgcctcgggccaggacccagggggccgccggagggcctgggccgagctgctggcgggaagggtcaagagggaaaaatataatcctgaaagggcacagaaattaaaggaatcagctgtgcgcctcctgcgaagccatcaggacctgaatgcccttttgcttgaggtagaaggtccactgtgtaaaaaattgtctctcagcaaagtgattgactgtgacagttctgaggcctatgctaatcattctagttcatttataggctctgctttgcaggatcaagcctcaaggctgggggttcccgtgggtattctctcagccgggatggttgcctctagcgtgggacagatctgcacggctccagcggagaccagtcaccctgtgctgctgactgtggagcagagaaagaagctgtcttccctgttagagtttgctcagtatttattggcacacagtatgttctcccgtctttccttctgtcaagaattatggaaaatacagagttctttgttgcttgaagcggtgtggcatcttcacgtacaaggcattgtgagcctgcaagagctgctggaaagccatcccgacatgcatgctgtgggatcgtggctcttcaggaatctgtgctgcctttgtgaacagatggaagcatcctgccagcatgctgacgtcgccagggccatgctttctgattttgttcaaatgtttgttttgaggggatttcagaaaaactcagatctgagaagaactgtggagcctgaaaaaatgccgcaggtcacggttgatgtactgcagagaatgctgatttttgcacttgacgctttggctgctggagtacaggaggagtcctccactcacaagatcgtgaggtgctggttcggagtgttcagtggacacacgcttggcagtgtaatttccacagatcctctgaagaggttcttcagtcataccctgactcagatactcactcacagccctgtgctgaaagcatctgatgctgttcagatgcagagagagtggagctttgcgcggacacaccctctgctcacctcactgtaccgcaggctctttgtgatgctgagtgcagaggagttggttggccatttgcaagaagttctggaaacgcaggaggttcactggcagagagtgctctcctttgtgtctgccctggttgtctgctttccagaagcgcagcagctgcttgaagactgggtggcgcgtttgatggcccaggcattcgagagctgccagctggacagcatggtcactgcgttcctggttgtgcgccaggcagcactggagggcccctctgcgttcctgtcatatgcagactggttcaaggcctcctttgggagcacacgaggctaccatggctgcagcaagaaggccctggtcttcctgtttacgttcttgtcagaactcgtgccttttgagtctccccggtacctgcaggtgcacattctccacccacccctggttcccagcaagtaccgctccctcctcacagactacatctcattggccaagacacggctggccgacctcaaggtttctatagaaaacatgggactctacgaggatttgtcatcagctggggacattactgagccccacagccaagctcttcaggatgttgaaaaggccatcatggtgtttgagcatacggggaacatcccagtcaccgtcatggaggccagcatattcaggaggccttactacgtgtcccacttcctccccgccctgctcacacctcgagtgctccccaaagtccctgactcccgtgtggcgtttatagagtctctgaagagagcagataaaatccccccatctctgtactccacctactgccaggcctgctctgctgctgaagagaagccagaagatgcagccctgggagtgagggcagaacccaactctgctgaggagcccctgggacagctcacagctgcactgggagagctgagagcctccatgacagaccccagccagcgtgatgttatatcggcacaggtggcagtgatttctgaaagactgagggctgtcctgggccacaatgaggatgacagcagcgttgagatatcaaagattcagctcagcatcaacacgccgagactggagccacgggaacacattgctgtggacctcctgctgacgtctttctgtcagaacctgatggctgcctccagtgtcgctcccccggagaggcagggtccctgggctgccctcttcgtgaggaccatgtgtggacgtgtgctccctgcagtgctcacccggctctgccagctgctccgtcaccagggcccgagcctgagtgccccacatgtgctggggttggctgccctggccgtgcacctgggtgagtccaggtctgcgctcccagaggtggatgtgggtcctcctgcacctggtgctggccttcctgtccctgcgctctttgacagcctcctgacctgtaggacgagggattccttgttcttctgcctgaaattttgtacagcagcaatttcttactctctctgcaagttttcttcccagtcacgagatactttgtgcagctgcttatctccaggccttattaaaaagtttcagttcctcatgttcagattgttctcagaggcccgacagcctctttctgaggaggacgtagccagcctttcctggagacccttgcaccttccttctgcagactggcagagagctgccctctctctctggacacacagaaccttccgagaggtgttgaaagaggaagatgttcacttaacttaccaagactggttacacctggagctggaaattcaacctgaagctgatgctctttcagatactgaacggcaggacttccaccagtgggcgatccatgagcactttctccctgagtcctcggcttcagggggctgtgacggagacctgcaggctgcgtgtaccattcttgtcaacgcactgatggatttccaccaaagctcaaggagttatgaccactcagaaaattctgatttggtctttggtggccgcacaggaaatgaggatattatttccagattgcaggagatggtagctgacctggagctgcagcaagacctcatagtgcctctcggccacaccccttcccaggagcacttcctctttgagattttccgcagacggctccaggctctgacaagcgggtggagcgtggctgccagccttcagagacagagggagctgctaatgtacaaacggatcctcctccgcctgccttcgtctgtcctctgcggcagcagcttccaggcagaacagcccatcactgccagatgcgagcagttcttccacttggtcaactctgagatgagaaacttctgctcccacggaggtgccctgacacaggacatcactgcccacttcttcaggggcctcctgaacgcctgtctgcggagcagagacccctccctgatggtcgacttcatactggccaagtgccagacgaaatgccccttaattttgacctctgctctggtgtggtggccgagcctggagcctgtgctgctctgccggtggaggagacactgccagagcccgctgccccgggaactgcagaagctacaagaaggccggcagtttgccagcgatttcctctcccctgaggctgcctccccagcacccaacccggactggctctcagctgctgcactgcactttgcgattcaacaagtcagggaagaaaacatcaggaagcagctaaagaagctggactgcgagagagaggagctattggttttccttttcttcttctccttgatgggcctgctgtcgtcacatctgacctcaaatagcaccacagacctgccaaaggctttccacgtttgtgcagcaatcctcgagtgtttagagaagaggaagatatcctggctggcactctttcagttgacagagagtgacctcaggctggggcggctcctcctccgtgtggccccggatcagcacaccaggctgctgcctttcgctttttacagtcttctctcctacttccatgaagacgcggccatcagggaagaggccttcctgcatgttgctgtggacatgtacttgaagctggtccagctcttcgtggctggggatacaagcacagtttcacctccagctggcaggagcctggagctcaagggtcagggcaaccccgtggaactgataacaaaagctcgtctttttctgctgcagttaatacctcggtgcccgaaaaagagcttctcacacgtggcagagctgctggctgatcgtggggactgcgacccagaggtgagcgccgccctccagagcagacagcaggctgcccctgacgctgacctgtcccaggagcctcatctcttctgatgagaattcgatatcaagcttatcgataccgtcgaatcccccgggctgcaggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagc

SEQ ID NO: 25 - protein, 1455 aa, Homo sapiens
MSDSWVPNSASGQDPGGRRRAWAELLAGRVKREKYNPERAQKLKESAVRLLRSHQDLNALLLEVEGPLCKKLSLSKVIDCDSSEAYANHSSSFIGSALQDQASRLGVPVGILSAGMVASSVGQICTAPAETSHPVLLTVEQRKKLSSLLEFAQYLLAHSMFSRLSFCQELWKIQSSLLLEAVWHLHVQGIVSLQELLESHPDMHAVGSWLFRNLCCLCEQMEASCQHADVARAMLSDFVQMFVLRGFQKNSDLRRTVEPEKMPQVTVDVLQRMLIFALDALAAGVQEESSTHKIVRCWFGVFSGHTLGSVISTDPLKRFFSHTLTQILTHSPVLKASDAVQMQREWSFARTHPLLTSLYRRLFVMLSAEELVGHLQEVLETQEVHWQRVLSFVSALVVCFPEAQQLLEDWVARLMAQAFESCQLDSMVTAFLVVRQAALEGPSAFLSYADWFKASFGSTRGYHGCSKKALVFLFTFLSELVPFESPRYLQVHILHPPLVPSKYRSLLTDYISLAKTRLADLKVSIENMGLYEDLSSAGDITEPHSQALQDVEKAIMVFEHTGNIPVTVMEASIFRRPYYVSHFLPALLTPRVLPKVPDSRVAFIESLKRADKIPPSLYSTYCQACSAAEEKPEDAALGVRAEPNSAEEPLGQLTAALGELRASMTDPSQRDVISAQVAVISERLRAVLGHNEDDSSVEISKIQLSINTPRLEPREHIAVDLLLTSFCQNLMAASSVAPPERQGPWAALFVRTMCGRVLPAVLTRLCQLLRHQGPSLSAPHVLGLAALAVHLGESRSALPEVDVGPPAPGAGLPVPALFDSLLTCRTRDSLFFCLKFCTAAISYSLCKFSSQSRDTLCSCLSPGLIKKFQFLMFRLFSEARQPLSEEDVASLSWRPLHLPSADWQRAALSLWTHRTFREVLKEEDVHLTYQDWLHLELEIQPEADALSDTERQDFHQWAIHEHFLPESSASGGCDGDLQAACTILVNALMDFHQSSRSYDHSENSDLVFGGRTGNEDIISRLQEMVADLELQQDLIVPLGHTPSQEHFLFEIFRRRLQALTSGWSVAASLQRQRELLMYKRILLRLPSSVLCGSSFQAEQPITARCEQFFHLVNSEMRNFCSHGGALTQDITAHFFRGLLNACLRSRDPSLMVDFILAKCQTKCPLILTSALVWWPSLEPVLLCRWRRHCQSPLPRELQKLQEGRQFASDFLSPEAASPAPNPDWLSAAALHFAIQQVREENIRKQLKKLDCEREELLVFLFFFSLMGLLSSHLTSNSTTDLPKAFHVCAAILECLEKRKISWLALFQLTESDLRLGRLLLRVAPDQHTRLLPFAFYSLLSYFHEDAAIREEAFLHVAVDMYLKLVQLFVAGDTSTVSPPAGRSLELKGQGNPVELITKARLFLLQLIPRCPKKSFSHVAELLADRGDCDPEVSAALQSRQQAAPDADLSQEPHLF